(19) United States
(12) Patent Application Publication
(10) Pub. No.: US 2022/0273571 A1
(43) Pub. Date: Sep. 1, 2022

(54) COMPOSITIONS OF PROBIOTICS AND BIOMASS AND METHODS FOR PROMOTING HEALTH IN A SUBJECT

(71) Applicant: Commonwealth Scientific and Industrial Research Organisation, Acton (AU)

(72) Inventors: Mary Ann AUGUSTIN, Acton (AU); Luz SANGUANSRI, Acton (AU); Michael CONLON, Acton (AU); Mya Myintzu HLAING, Acton (AU)

(21) Appl. No.: 17/633,064

(22) PCT Filed: Aug. 6, 2020
(86) PCT No.: PCT/AU2020/050811

§ 371 Date: Feb. 4, 2022

(30) Foreign Application Priority Data
Aug. 7, 2019 (AU) 2019902828

(51) Int. Cl. A61K 9/14; A61K 35/747; A61K 35/742; A61K 35/741; A61K 35/745; A61K 35/744; A61K 31/202; A61K 47/44; A61K 47/46; A61K 31/201; A61P 1/14; A61K 9/50

(52) U.S. Cl. CPC A61K 9/14; A61K 35/747; A61K 35/742; A61K 35/741; A61K 35/745; A61K 35/744; A61K 31/202; A61K 47/44; A61K 47/46; A61K 31/201; A61P 1/14; A61K 9/5089; A61K 2035/115

(57) ABSTRACT

The present invention provides compositions and products comprising a probiotic entrapped or encapsulated in a protein and carbohydrate matrix, sourced from a single non-fermented biomass and methods of producing thereof. In addition, the present invention provides methods for promoting health in a subject comprising administering a probiotic to a subject.

### FIELD OF THE INVENTION

The present invention provides compositions and products comprising a probiotic entrapped or encapsulated in a matrix and methods of producing thereof. In addition, the present invention provides methods for promoting health in a subject comprising administering a probiotic to a subject.

### BACKGROUND OF THE INVENTION

The prevalence of chronic diseases such as obesity, cardiovascular disease, metabolic syndrome, inflammatory diseases, autoimmune diseases, diabetes, gut health conditions and certain cancers is increasing globally, fuelled particularly by dramatic rises in developing countries where growing affluence is also associated with an expanding adoption of more Westernised diet and lifestyle patterns. While over consumption of high calorie, easily digested foods and beverages plays an important role, there is growing evidence that changes to the 10¹⁴ microbes, comprising over 10³ bacterial species (collectively the gut microbiota) of the human large bowel, driven by these dietary patterns also makes a contribution and provides target for preventive and clinical intervention. Diet plays a big part in feeding this hungry gut microbiota, shaping both its structure (the relative proportions of the different species) and its function (genes expressed, metabolites made and their interaction with the subject). Dietary fibre is fermented by the gut microbiota into short chain fatty acid/s (SCFA). SCFA positively influence the gastrointestinal microenvironment (increases gut health) and other organ sites in the health as they are small enough to enter the blood stream and can be distributed to other sites in the body.

Accordingly, there is a requirement for supplements and nutritional agents that promote health, including gut health, in a subject.

### SUMMARY OF THE INVENTION

The present inventors have developed compositions and products comprising a probiotic entrapped or encapsulated in a matrix and methods of producing thereof. The present inventors have also developed methods for promoting health in a subject comprising delivering a probiotic to a subject.

In an aspect, the present invention provides a powder composition comprising a probiotic entrapped or encapsulated in a matrix comprising protein and carbohydrate from a non-fermented biomass from a single species of organism.

In an embodiment, the entrapped or encapsulated probiotic has higher viability compared to the unentrapped or unencapsulated probiotic.

In an embodiment, the composition is synbiotic.

In an aspect, the present invention provides a method of producing a powder composition, the method comprising

i) producing an aqueous mixture comprising
a) protein and carbohydrate from a non-fermented biomass from a single species of organism, and
b) a probiotic, and

ii) forming, from the mixture, a powder comprising the probiotic entrapped or encapsulated in a matrix comprising the protein and the carbohydrate.

In an embodiment, the method further comprises adding oil to the aqueous mixture.

In an aspect, the present invention provides a product comprising the composition as described herein, or produced by the method as described herein.

In an embodiment, the product is a synbiotic.

In an aspect, the present invention provides a method of promoting health in a subject, comprising administering to the subject a composition as described herein, or product as described herein.

In an aspect, the present invention provides a method of promoting the health of the gut microbiome in a subject, comprising administering to the subject the composition as described herein, or product as described herein.

In an aspect, the present invention provides a method of treating and/or preventing microbial dysbiosis in the gastrointestinal tract of a subject, comprising administering to the subject the composition as described herein, or product as described herein.

In an aspect, the present invention provides a method of treating and/or preventing inflammation in a subject, comprising administering to the subject the composition as described herein, or product as described herein.

In an aspect, the present invention provides a method of treating and/or preventing diabetes in a subject, comprising administering to the subject the composition as described herein, or product as described herein.

In an aspect, the present invention provides a method of promoting growth or feed efficacy in livestock comprising administering to a livestock the composition as described herein, or the product as described herein.

In an aspect, the present invention provides a method of improving the quality of livestock derived products comprising administering to a livestock the composition as described herein, or the product as described herein.

In an aspect, the present invention provides use of a composition as described herein, or product as described herein in the manufacture of a medicament for promoting health in a subject.

In an aspect, the present invention provides use of a composition as described herein, or product as described herein in the manufacture of a medicament for promoting health of the gut microbiome in a subject.

In an aspect, the present invention provides use of a composition as described herein, or product as described herein in the manufacture of a medicament for treating and/or preventing microbial dysbiosis in the gastrointestinal tract of a subject.

In an aspect, the present invention provides use of a composition as described herein, or product as described herein in the manufacture of a medicament for treating and/or preventing inflammation in a subject.

In an aspect, the present invention provides use of a composition as described herein, or product as described herein in the manufacture of a medicament for treating and/or preventing diabetes in a subject.

In an aspect, the present invention provides a pharmaceutical composition comprising a composition as described herein, or product as described herein for use in promoting health in a subject.

In an aspect, the present invention provides a pharmaceutical composition comprising a composition as described herein, or product as described herein for use in promoting health of the gut microbiome in a subject.

In an aspect, the present invention provides a pharmaceutical composition comprising a composition as described herein, or product as described herein for treating and/or preventing microbial dysbiosis in the gastrointestinal tract of a subject.

In an aspect, the present invention provides a pharmaceutical composition comprising a composition as described herein, or product as described herein for treating and/or preventing inflammation in a subject.

In an aspect, the present invention provides a pharmaceutical composition comprising a composition as described herein, or product as described herein for treating and/or preventing diabetes in a subject.

In an aspect, the present invention provides a faecal microbiota suitable for transplantation into a subject, wherein the faecal microbiota has been isolated from a subject administered a composition as described herein, or a product as described herein.

Any embodiment herein shall be taken to apply mutatis mutandis to any other embodiment unless specifically stated otherwise. For instance, as the skilled person would understand examples of a probiotic outlined above for the compositions of the invention equally apply to products and methods of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANING DRAWINGS

FIG. 1. Shows a flowchart for the preparation of broccoli matrices. Broccoli puree (BP), broccoli stems and leaves (BSL), epigallocatechin gallate (EGCG).

FIG. 2. Shows the concentrations of acetic acid, propionic acid, butyric acid and total short chain fatty acid (μmol/mL±STD) in the in vitro colonic fermentations of control samples and formulations comprising puree made from broccoli stems and leaves (BSL) at 0 hr and 24 hrs post-fermentation with one of the following formulations: no substrate, cellulose, inulin, puree made from broccoli stems and leaves (BSL), tuna oil (TO), epigallocatechin gallate (EGCG), BSL-TO, BSL-EGCG, BSL-EGCG-TO.

FIG. 3. Shows the concentrations of acetic acid, propionic acid, butyric acid and total short chain fatty acid (μmol/mL±STD) in the in vitro colonic fermentations of control samples and formulations comprising broccoli puree (made from whole broccoli heads) or skim milk powder at 0 hr and 24 hrs post-fermentation. with one of the following formulations: no substrate, cellulose (cell), inulin (positive control), algal oil (AO), broccoli puree made from whole broccoli heads (BP), Lactobacillus rhamnosus GG (LGG), broccoli puree+algal oil (BP-AO), broccoli puree+Lactobacillus rhamnosus LGG (BP-LGG), broccoli puree+Lactobacillus rhamnosus LGG with pH adjustment (BP-LGG-pHAdjust), broccoli puree+algal oil+Lactobacillus rhamnosus LGG (BP-AO-LGG) or broccoli puree+algal oil+Lactobacillus rhamnosus LGG with pH adjustment (BP-AO-LGG-pHAdjust).

FIGS. 4-12. Show the relative abundances of Colinsella, Bacteriodes, Parabacteroides, Paraprevotella, Bacillus, Lactobacillus, Turicibacter, Christensenellaceae, Clostridiales-other, Clostridium, Lachnospiraceae-other, Coprococcus, Dorea, Lachnospira, Roseburia, Ruminococcaceae-other, Faecalibacterium, Oscillospira, Ruminococcos, Dialister, Veillonella, Erysipelotrichaceae-other, Klebsiella, and Trabulsiella after 24 h fermentation post treatment with one of the following formulations: tuna oil (TO), broccoli stems and leaves (BSL), BSL-TO, BSL-Epigallocatechin gallate (EGCG), BSL-EGCG-TO, BSL-TO, cellulose, ECGC, inulin (positive control), Lactobacillus rhamnosus GG (LGG), no substrate, skim milk powder (SMP).

FIGS. 13-21. Show the relative abundances of Colinsella, Bacteriodes, Parabacteroides, Paraprevotella, Bacillus, Lactobacillus, Turicibacter, Christensenellaceae, Clostridiales-other, Clostridium, Lachnospiraceae-other, Coprococcus, Dorea, Lachnospira, Roseburia, Ruminococcaceae-other, Faecalibacterium, Oscillospira, Ruminococcos, Dialister, Veillonella, Erysipelotrichaceae-other, Klebsiella, and Trabulsiella after 24 h fermentation post treatment with one of the following formulations: no substrate, cellulose (cell), inulin (positive control), algal oil (AO), broccoli puree made from whole broccoli heads (BP), Lactobacillus rhamnosus GG (LGG), broccoli puree+algal oil (BP-AO), broccoli puree+Lactobacillus rhamnosus LGG (BP-LGG), broccoli puree+Lactobacillus rhamnosus LGG with pH adjustment (BP-LGG-pHAdjust), broccoli puree+algal oil+Lactobacillus rhamnosus LGG BP-AO-LGG or broccoli puree+algal oil+Lactobacillus rhamnosus with pH adjustment (BP-AO-LGG-pHAdjust).

FIGS. 22. Shows the protection of broccoli matrix with Lactobacillus rhamnosus GG (LGG) on stability of algal oil against oxidation.

### DETAILED DESCRIPTION

### General Techniques and Definitions

Unless specifically defined otherwise, all technical and scientific terms used herein shall be taken to have the same meaning as commonly understood by one of ordinary skill in the art (e.g., enzyme, fermentation, microbiome).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about”, unless stated to the contrary, refers to ±10%, more preferably ±5%, even more preferably ±1%, of the designated value.

As used herein “component” refers to a part or element of a larger whole.

As used herein “protein” or “polypeptide” refers to macromolecules comprising carbon, hydrogen, oxygen, nitrogen and usually sulfur comprising polymers of amino acids linked together by peptide binds.

As used herein “carbohydrate” refers to a class of molecules of the general formula Cx(H2O)y. In an embodiment, the carbohydrate from a Brassicaceae, Musaceae, Convolvulaceae, Umbelliferae, Asparagaceae, Arecaceae, Myrtaceae, Rosaceae, Musaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae or Caesalpinioideae. In an embodiment, the carbohydrate is from a Brassicaceae, Musaceae, or Convolvulaceae.

As used herein, “entrapment” or “entrapped” or “entrapping” refers to binding or partitioning of a probiotic or oil to one or more components of the biomass or matrix as described herein. In an embodiment, the component is carbohydrate or a protein. In an embodiment, the component is fibre. In an embodiment, the component is a prebiotic. In an embodiment, an entrapped probiotic has a higher viability compared to the unentrapped probiotic. In an embodiment, an entrapped probiotic has a higher viability compared to the unentrapped probiotic when stored at about 24° C. to about 40° C. In an embodiment, entrapment increases the resistance of the oil to one or more of degradation by oxygen, temperature, pH, moisture, light and pro-oxidants.

As used herein, “encapsulation” or “encapsulated” refers to forming of a functional barrier (a matrix) around a probiotic and/or oil. In an embodiment, the functional barrier comprises protein and carbohydrate from biomass as described herein. In an embodiment, the functional barrier comprises a fibre from the biomass as described herein. In an embodiment, the functional barrier comprises a prebiotic from the biomass as described herein. In an embodiment, an encapsulated probiotic has a higher viability compared to the unencapsulated probiotic. In an embodiment, an entrapped probiotic has a higher viability compared to the unentrapped probiotic when stored at about 24° C. to about 40° C. In an embodiment, the functional barrier increases the resistance of oil to one or more of degradation by oxygen, temperature, pH, moisture and light. In an embodiment, the probiotic is in the oil when the oil is encapsulated. In an embodiment, encapsulation increases the resistance of the oil to one or more of degradation by oxygen, temperature, pH, moisture, light and pro-oxidants.

In an embodiment, encapsulation as described herein comprises a probiotic and/or oil which is/are present in various assemblies/entities surrounded by the major structural components (i.e. protein, soluble and insoluble fibre, other high/low molecular weight carbohydrates including starch and sugars, fat) in the matrix. The structural assemblies/entities which contain the probiotic and/or oil include for example: an oil globule surrounded by a protein component at the interface in a core-and-shell structure; oil and/or probiotic bound to the fibre/starch component in the matrix or dispersed/embedded within the matrix; a probiotic and/or oil globule surrounded by the matrix components; a probiotic and/or oil dispersed in a continuous network of matrix; oil/probiotic bound to components in the matrix and oil present as globules surrounded by the matrix component; or a combination of the above structures carried within the matrix.

As used herein, the “matrix” refers to a functional barrier comprising protein and carbohydrate from a non-fermented biomass from a single species of organism. In an embodiment, the matrix comprises sulforaphane. In an embodiment, the matrix comprises a glucosinolate. In an embodiment, the matrix comprises glucoraphanin. In an embodiment, the components of the matrix are food grade. In an embodiment, the matrix comprises a whole vegetable biomass. In an embodiment, the matrix comprises a whole fruit biomass. In an embodiment, the matrix comprises about 50% to about 100% vegetable and/or fruit material. In an embodiment, the matrix comprises about 60% to about 100% vegetable and/or fruit material. In an embodiment, the matrix comprises about 70% to about 100% vegetable and/or fruit material. In an embodiment, the matrix comprises about 80% to about 100% vegetable and/or fruit material. In an embodiment, the matrix comprises about 85% to about 100% vegetable and/or fruit material. In an embodiment, the matrix comprises about 90% to about 100% vegetable and/or fruit material. In an embodiment, the matrix comprises about 95% to about 100% vegetable and/or fruit material. In an embodiment, the matrix is free from dairy products. In an embodiment, the matrix is free from animal products. In an embodiment, the matric is vegan.

As used herein, “viability” refers to the probiotics ability to survive or live successfully.

As used herein, “higher” refers to an increased or greater amount compared to a control. In an embodiment, higher refers to an increase of about 5% to about 100%, or about 10% to 100%, or about 15% to about 100%, or about 20% to about 100%, or about 30% to about 100%, or about 40% to about 100%, or about 50% to about 100%, or about 60% to about 100%, or about 70% to about 100%, or about 80% to about 100%, or about 90% to about 100%.

As used herein, “species of organism” refers to a subdivision of a genus. In an embodiment, “species of organism” refers to a group of organisms consisting of individuals capable of breeding among themselves.

As used herein, the term “subject” is any animal. In one example, the animal is a vertebrate. For example, the animal is a mammal, avian, arthropod, chordate, amphibian or reptile. In one embodiment, the subject is a human. In one embodiment, the subject is livestock (e.g. sheep, cow, goat, chicken, turkey, horse, donkey, pig, fish, prawn, shrimp). In one embodiment, the livestock is an aquatic livestock. In an embodiment, the aquatic livestock is selected from: fish molluscs, crustaceans, prawn and shrimp. In an embodiment, the fish is selected from salmon, carp, tilapia, trout and catfish. In one embodiment, the subject is a companion animal. Exemplary subjects include but are not limited to human, fish, prawns, primate, livestock, companion animals (e.g. dogs, cats), laboratory test animals (e.g. mice, rabbits, rats, guinea pigs, hamsters), captive wild animal (e.g. fox, deer).

As used herein, the terms “treating” or “treatment” include administering a effective amount of a product or composition as described herein sufficient to reduce or eliminate at least one symptom of a specified disease or condition.

As used herein, the terms “preventing” or “prevent” include administering a effective amount of a product or composition as described herein sufficient to stop or hinder the development of at least one symptom of a specified disease or condition.

As used herein, “microbiome” refers to the microorganisms in a particular environment which can include the body or a part of the body of a subject. For example, the gut microbiome refers to the community of microorganism in the gut.

As used herein, “microorganism” or “microorganisms” refers to microscopic organisms including bacterial, viral, fungal or eukaryotic organisms.

As used herein, “gastrointestinal tract” refers to at least a portion of the gastrointestinal tract. In an embodiment, the portion of the gastrointestinal tract is selected from a portion of the oral cavity, salivary glands, oesophagus, stomach, small intestine, duodenum, jejunum, ileum, large intestine, colon, rectum, cecum, anus and appendix. In an embodiment, the portion of the gastrointestinal tract is the large intestine. In an embodiment, the portion of the gastrointestinal tract is the colon.

As used herein, the “upper gastrointestinal tract” comprises at least a portion of the upper gastrointestinal tract selected from the oral cavity, salivary glands, oesophagus, stomach, small intestine, duodenum, jejunum and ileum.

As used herein, the “lower gastrointestinal tract” comprises at least a portion of the lower gastrointestinal tract selected from the large intestine, colon, rectum, cecum, anus and appendix.

### Probiotic

The compositions or products as described herein comprise a probiotic. As used herein, a “probiotic” refers to live microorganism which when administered in an adequate amount confers a health benefit to a subject as described herein.

In an embodiment, the health benefit is an increase in one or more beneficial bacteria in the gut microflora. As described herein a “beneficial bacterial” confers a health benefit to a subject (host). In an embodiment, the beneficial bacteria is a lactic acid bacteria. In an embodiment, the beneficial bacteria is a bacteria that produces one or more short chain fatty acid/s (SFCA).

In an embodiment, the health benefit is a decrease in one or more non-beneficial bacteria in the gut microflora. In an embodiment, the non-beneficial bacteria is selected from a pathogenic Escherichia coli, Bacteroides and Parabacteriodes. In an embodiment, the non-beneficial bacteria is a pathogenic Escherichia coli. In an embodiment, the non-beneficial bacteria is a pathogenic Bacteroides. In an embodiment, the non-beneficial bacteria is a pathogenic Parabacteriodes.

In an embodiment, the health benefit is an increase in lactic acid bacteria. In an embodiment, the health benefit is an increase in a bacteria that assists with the production of lactic acid. In an embodiment, the health benefit is an increase in a bacteria that produces one or more SCFA. In an embodiment, the bacteria that produces one or more SCFA is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus, Clostridium cluster XIVa. Bifidobacterium spp., Bacteroidetes and Negativicutes classes of Firmicutes. In an embodiment, the bacteria that produces one or more SCFA produces butyrate and is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus and Clostridium cluster XIVa. In an embodiment, the bacteria that produces one or more SCFA produces acetate and is Bifidobacterium spp. In an embodiment, the bacteria that produces one or more SCFA produces propionate and is selected from Bacteroidetes and Negativicutes classes of Firmicutes.

In an embodiment, the health benefit is an increase in a bacteria that assists with the production of one of more SCFA.

In an embodiment, the health benefit is an increase in the resistance of the gut microbiome. In an embodiment, the health benefit is an increase in the resilience of the gut microbiome. In an embodiment, the health benefit is a reduction in gut leakiness. In an embodiment, the health benefit is the increase the production of one or more SCFA in the gastrointestinal tract.

In an embodiment, the probiotic is selected from one or more or all of: lactic acid bacteria, Bifidobacteria, Bacteroidetes, Baciullus, Streptococcus, Escherichia, Enterococcus, and Saccharomyces.

In an embodiment, the probiotic is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermenturn, Lactobacillus gasseri, Lactobacillus johnsonii, Lactobacillus lactis, Lactobacillus paracasei, Lactobacillus reuteri, Pediococcus pentosaceus and Pedicoccus acidilacti.

In an embodiment, the lactic acid bacteria is selected from one or more or all of: i) BF1 deposited under V17/021729 on 25 Sep. 2017 at the National Measurement Institute 1/153 Bertie Street, Port Melbourne, Victoria, Australia 3207; ii) BF2 deposited under V17/021730 on 25 Sep. 2017 at the National Measurement Institute 1/153 Bertie Street, Port Melbourne, Victoria, Australia 3207; iii) B1 deposited under V17/021731 on 25 Sep. 2017 at the National Measurement Institute 1/153 Bertie Street, Port Melbourne, Victoria, Australia 3207; iv) B2 deposited under V17/021732 on 25 Sep. 2017 at the National Measurement Institute 1/153 Bertie Street, Port Melbourne, Victoria, Australia 3207; v) B3 deposited under V17/021733 on 25 Sep. 2017 at the National Measurement Institute 1/153 Bertie Street, Port Melbourne, Victoria, Australia 3207; vi) B4 deposited under V17/021734 on 25 Sep. 2017 at the National Measurement Institute 1/153 Bertie Street, Port Melbourne, Victoria, Australia 3207; and vii) B5 deposited under V17/021735 on 25 Sep. 2017 at the National Measurement Institute 1/153 Bertie Street, Port Melbourne, Victoria, Australia 3207.

In embodiment, the Bifidobacteria is selected from one or more of: Bifidobacteria adolescentis, Bifidobacteria animalis, Bifidobacteria bifidum, Bifidobacteria breve, Bifidobacteria infantis, Bifidobacteria longum, and Bifidobacteria thermophilum.

In embodiment, the Baciullus is selected from one or more of: Baciullus cereus, Baciullus clausii, Baciullus coagulans, Baciullus licheniformis, Baciullus pumulis and Baciullus subtilis.

In embodiment, the Streptococcus is Streptococcus thermophiles. In embodiment, the Escherichia is beneficial strain of Escherichia coli.

In embodiment, the Enterococcus is Enterociccus faecium.

In embodiment, the Saccharomyces is Saccharomyces cerevisiae.

In an embodiment, the lactic acid bacteria was isolated from a Brassica oleracea. In an embodiment, the lactic acid bacteria was isolated from broccoli. A person skilled in the art will appreciate that this includes direct isolation or indirect isolation (e.g. isolated from an original source and cultivated a number of passages, optionally cryogenically stored, before use). In an embodiment, the lactic acid bacteria was isolated from Australian broccoli. In an embodiment, the lactic acid bacteria is selected from: i) a Leuconostoc mesenteroides; ii) a Lactobacillus plantarum; iii) a Lactobacillus pentosus; iv) a Lactobacillus rhamnosus; v) a combination of i) and ii); vi) a combination of i), ii) and iii); and vii) a combination of i), ii) and iv). In one embodiment, the lactic acid bacteria is selected from one or more or all of BF1, BF2, B1, B2, B3, B4 and B5. In an embodiment, the lactic acid bacteria is B1. In an embodiment, the lactic acid bacteria is B2. In an embodiment, the lactic acid bacteria is B3. In an embodiment, the lactic acid bacteria is B4. In an embodiment, the lactic acid bacteria is B5. In an embodiment, the probiotic is a tablet, powder or liquid.

In an embodiment, the probiotic is Faecalibacterium prausnitzii. In an embodiment, the probiotic is Akkermansia muciniphila.

In an embodiment, the Brassicaceae product as described herein comprises a combined prebiotic and probiotic.

In an embodiment, the probiotic is suitable for use in humans, including for example, the probiotics described in Markowiak and Śliżewska, (2017).

In an embodiment, the probiotic is suitable for use in livestock, including for example, probiotics described in Markowiak and Śliżewska (2018) and Chaucheyras-Durand and Durand (2010).

In an embodiment, the probiotic suitable for use in livestock is selected from one or more of: Lactobacillus, Bifidobacterium, Bacillus, Enterococcus, Lactococcus, Leuconostoc, Pediococcus, Streptococcus, Sporolactobacillus, Saccharomyces cerevisiae and Kluyveromyces.

In an embodiment, the Lactobacillus is selected from one or more of: Lactobacillus brevisa, Lactobacillus caseia, Lactobacillus crispatusa, Lactobacillus farciminisa, Lactobacillus fermentuma, Lactobacillus murinus, Lactobacillus gallinariuma, Lactobacillus paracaseia, Lactobacillus pentosusa, Lactobacillus plantaruma, Lactobacillus reuteria, Lactobacillus rhamnosusa and Lactobacillus salivariusa.

In an embodiment, the Bifidobacterium is selected from one or more of: Bifidobacterium animalisa, Bifidobacterium longuma, Bifidobacterium pseudolongum and Bifidobacterium thermophilum.

In an embodiment, the Bacillus is selected from one or more of: Bacillus cereus, Bacillus licheniformisa and Bacillus subtilisa

In an embodiment, the Enterococcus is selected from one or more of: Enterococcus faecalis and Enterococcus faecium,

In an embodiment, the Lactococcus is Lactococcus lactisa.

In an embodiment, the Leuconostoc is selected from one or more of: Leuconostoc citreuma, Leuconostoc lactisa and Leuconostoc mesenteroidesa.

In an embodiment, the Pediococcus is selected from one or more of: Pediococcus acidilacticia and Pediococcus pentosaceusa.

In an embodiment, the Streptococcus is selected from one or more of: Streptococcus infantarius, Streptococcus salivarius, and Streptococcus thermophilusa.

In an embodiment, the Sporolactobacillus in Sporolactobacillus inulinus

In an embodiment, the Saccharomyces is selected from one or more of: Saccharomyces cerevisiae and Saccharomyces pastorianusa.

In an embodiment, the Kluyveromyces is selected from one or more of: Kluyveromyces fragilis and Kluyveromyces marxianusa.

In an embodiment, the Aspergillus is selected from one or more of: Aspergillus orizae and Aspergillus niger.

In an embodiment, the probiotic is suitable for use in aquaculture as described herein, including for example, the probiotics described Martínez Cruz et al. (2012) and Huynh et al. (2017). In aquaculture probiotics are useful for growth promotion, inhibition of pathogens, nutrient absorption, nutrient utilization, and improvement of water quality.

In an embodiment, the probiotic is not Lactobacillus acidophilus. In an embodiment, the probiotic is not Lactobacillus casei.

### Synbiotic

In an embodiment, the composition or product as described herein comprises a prebiotic and a probiotic which are synbiotic.

As used herein, a “synbiotic” refers to a composition comprising a prebiotic and probiotic which results in a synergistic effect. In an embodiment, the prebiotic assists the probiotic with survival. In an embodiment, the prebiotic protects the probiotic from damage caused by external environmental factors e.g. pH or digestive enzymes. In an embodiment, the prebiotic is a food source to aid viable and/or growth of the probiotic. In an embodiment, the prebiotic improves the shelf life of a live microorganism. In an embodiment, a prebiotic improves the delivery of a live microorganism (e.g. passage of the upper gastrointestinal tract). In an embodiment, a prebiotic improves the survival of live microorganism. In an embodiment, a prebiotic improves the survival of live microorganism in the lower gastrointestinal tract (e.g. by providing a preferred food source for metabolism by the microorganism). In an embodiment, the composition or product as described herein comprises a prebiotic and a probiotic which are synbiotic. In an embodiment, the synbiotic is suitable for you in humans. In an embodiment, the synbiotic is suitable for use in livestock. In an embodiment, the livestock is aquaculture. In an embodiment, the synbiotic is suitable for use in companion animals.

### Prebiotic

An added advantage of the compositions or products as described herein is that they comprise a prebiotic. As used herein a “prebiotic” refers to a group of nutrients that are degraded by the gut microbiota. Prebiotics result in changes in the composition and/or activity of the gastrointestinal microbiota conferring benefits upon the health of the host (e.g. gut health).

In an embodiment, the prebiotic is degraded into one or more short chain fatty acid/s including salts or esters thereof (SCFA). SCFA positively influence the gastrointestinal microenvironment (increase gut health) and distal organ sites as they are small enough to enter the blood and can be delivered to e.g. the central nervous system, immune system, respiratory and cardiovascular system.

In an embodiment, the prebiotic increases health of the subject by increasing the level of one or more SCFA in the gastrointestinal tract of the subject. In an embodiment, the prebiotic increases health of the subject by increasing the level of one or more SCFA in the colon of the subject.

In an embodiment, the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoic acid), isobutyric acid (2-methylpropanoic acid), valeric acid (pentanoic acid), isovaleric acid (3-methylbutanoic acid), and caproic acid (hexanoic acid).

In an embodiment, the SCFA is selected from one or more or all of: butyrate, propionate, and acetate. In an embodiment, the SCFA is butyrate. In an embodiment, the SCFA is propionate. In an embodiment, the SCFA is acetate. As used herein, the term “total SCFA” refers to the combination of butyrate, propionate and acetate.

### Biomass

The present invention relates, to a composition or product and methods for producing a composition or product from biomass comprising protein and carbohydrate from a single (first) species of organism. The protein and carbohydrate have not been separated from each other before being used in a method of the invention.

Whilst in some embodiments the whole biomass can be used, in other embodiments the biomass has been processed to remove, or reduce the concentration of one or more components of the biomass. In an embodiment, less than about 50% of the biomass is removed before being used in a method of the invention. In an embodiment, less than about 40% of the biomass is removed before being used in a method of the invention. In an embodiment, less than about 30% of the biomass is removed before being used in a method of the invention. In an embodiment, less than about 20% of the biomass is removed before being used in a method of the invention. In an embodiment, less than about 10% of the biomass is removed before being used in a method of the invention. In an embodiment, less than about 5% of the biomass is removed before being used in a method of the invention. In an embodiment, less than about 1% of the biomass is removed before being used in a method of the invention. In an embodiment, none of the biomass is removed before being used in a method of the invention.

In an embodiment, the biomass is a vegetable and the whole vegetable is used.

In an embodiment, the biomass is a fruit and the whole fruit is used.

In an embodiment, the biomass is dried or concentrated to remove water. In an embodiment, drying removes about 60% to about 90% of the weight of the biomass. In an embodiment, drying removes about 70% to about 90% of the weight of the biomass. In an embodiment, drying removes about 80% to about 90% of the weight of the biomass.

In an embodiment, the biomass comprises protein and carbohydrate from a single species of organism only (no protein or carbohydrate from a further species of organism). In an embodiment, the biomass further comprises protein and carbohydrate from one or more further species of organism (e.g. second, third, fourth, fifth etc. species of organism). As with the biomass from the first species of organism, the protein and carbohydrate from the further species has not been separated from each other before being used in a method of the invention. In an embodiment, the biomass further comprises protein and carbohydrate from a second species of organism. In an embodiment, the biomass further comprises protein and carbohydrate from a second and a third species of organism. In an embodiment, the biomass further comprises protein and carbohydrate from a second, third and fourth species of organism.

In an embodiment, the biomass and/or further biomass comprises fibre which has not been separated from the protein and carbohydrate of the biomass and/or further biomass.

In an embodiment, the biomass and/or further biomass comprises a prebiotic.

In an embodiment, the biomass and/or further biomass comprises catechins which have not been separated from the protein and carbohydrate of the biomass and/or further biomass.

In an embodiment, the biomass and/or further biomass has a protein to carbohydrate ratio of between 1:1 and 1:20. In an embodiment, the biomass and/or further biomass has a protein to carbohydrate ratio of between 1:1 and 1:10.5. In an embodiment, the biomass and/or further biomass has a protein to carbohydrate ratio of between about 1:4.5 and 4:1. In an embodiment, the biomass and/or further biomass has a protein to carbohydrate ratio of between about 1:2.5 and 2:1. In an embodiment, the biomass and/or further biomass has a protein to carbohydrate ratio of about 1:2.4. In an embodiment, the biomass and/or further biomass additionally comprises fiber. In an embodiment, the biomass or further biomass has a protein to carbohydrate ratio as shown in Table 1.

In an embodiment, the biomass or further biomass is green tea leaf powder (matcha). In an embodiment, matcha, (˜2% moisture) comprises about 35.5% protein, about 39.6% carbohydrate, about 5.9% fat and about 6.0% fat on a dry basis. In an embodiment, matcha comprises about 13.1% catechins.

In an embodiment, protein is added to the biomass and/or further biomass to form a protein to carbohydrate ratio of between about 1:1 and 1:10.5. In an embodiment, protein is added to the biomass and/or further biomass to form a protein to carbohydrate ratio of between about 1:1 and 1:14. In an embodiment, protein is added to the biomass and/or further biomass to form a protein to carbohydrate ratio of between about 1:1 and 1:19.5 In an embodiment, protein is added to the biomass and/or further biomass to form a protein to carbohydrate ratio of between about 1:4.5 and 4:1. In an embodiment, protein is added to the biomass and/or further biomass to form a protein to carbohydrate ratio of between about 1:2.5 and 2:1.

In an embodiment, carbohydrate is added to the biomass and/or further biomass to form a protein to carbohydrate ratio of between about 1:1 and 1:10.5. In an embodiment, carbohydrate is added to the biomass and/or further biomass to form a protein to carbohydrate ratio of between about 1:1 and 1:14. In an embodiment, carbohydrate is added to the biomass and/or further biomass to form a protein to carbohydrate ratio of between about 1:1 and 1:19.5. In an embodiment, carbohydrate is added to the biomass and/or further biomass to form a protein to carbohydrate ratio of between about 1:4.5 and 4:1. In an embodiment, carbohydrate is added to the biomass and/or further biomass to form a protein to carbohydrate ratio of between about 1:2.5 and 2:1.

TABLE 1
Protein (P) and carbohydrate (CHO) ratio of select fresh biomasses and biomass flours

| | Protein | | | | | P:CHO |
|---|---|---|---|---|---|---|
| Vegetable | (P) | CHO | Fat | Minerals | Moisture | ratio |
| | in fresh | | | | | |
| Garlic | 6.36% | 33.06% | 0.50% | 0.78% | 59.30% | 1:5.2 |
| Onion | 1.10% | 9.34% | 0.10% | 0.21% | 89.25% | 1:8.5 |
| Mushroom | 2.50% | 4.30% | 0.10% | 0.60% | 92.50% | 1:1.7 |
| Spinach | 2.90% | 3.60% | 0.40% | 0.87% | 92.23% | 1:1.2 |
| Kale | 4.30% | 8.80% | 0.90% | 0.82% | 85.18% | 1:2.1 |
| Snow peas | 2.80% | 7.55% | 0.20% | 0.33% | 89.12% | 1:2.7 |
| Asparagus | 2.20% | 3.88% | 0.12% | 0.30% | 93.50% | 1:1.8 |
| Tomatoes | 0.90% | 3.90% | 0.20% | 0.27% | 94.79% | 1:4.3 |
| Avocado | 2.00% | 8.53% | 14.66% | 0.59% | 74.22% | 1:4.3 |
| Carrots | 0.93% | 9.60% | 0.24% | 0.47% | 88.76% | 1:10.3 |
| Broccoli | 2.82% | 6.64% | 0.37% | 0.48% | 89.69% | 1:2.4 |
| Artichoke | 2.89% | 11.39% | 0.34% | 0.72% | 84.66% | 1:3.9 |
| Cauliflower | 1.90% | 5.00% | 0.30% | 0.33% | 92.47% | 1:2.6 |
| Brussel sprouts | 3.40% | 9.00% | 0.30% | 0.41% | 86.89% | 1:2.7 |
| | In flours (expressed at g/100 g dry matter) | | | | | |
| Ripe Banana flour | 5.52% | 80.41% | 3.67% | 2.27% | 8.63% | 1:14.6 |
| (Ripening Stage 4) | | (52% sugar) | | | | |
| Unripe Banana | 3.69% | 82.34% | 1.33% | 2.10% | 10.88% | 1:22.3 |
| flour (Ripening | | (6.33% sugar) | | | | |
| Stage 1sweet | | | | | | |
| potatp flour | | | | | | |
| | In commercial flours (g/100 g ingredient) | | | | | |
| Green banana | 4.0% | 77.1% | <1.0% | 1.64% | 6.2% | 1:19.3 |
| flour | | | | | | |
| Sweet potato flour | 3.7% | 51.7% | 2.1% | 0.02% | 6.41% | 1:14.0 |

As used herein, ripe banana refers to banana that is at Ripening Stage 4.. In green banana, a high proportion of the carbohydrate component is starch whereas in ripe banana most of the carbohydrate is in the form of sugars due to conversion of starch to sugars during the ripening process.

In an embodiment, the biomass is the entire organism or one or more parts thereof.

In an embodiment, the biomass and/or further biomass comprises the whole biomass (or a piece thereof) in fresh/raw or dried form. In an embodiment, the biomass and/or further biomass is fresh/raw. In an embodiment, the biomass and/or further biomass is pre-treated as described herein.

In an embodiment, the biomass and/or further biomass is a product of an extraction or separation process as described herein suitable for removing one or more component/s from the biomass and/or further biomass.

In an embodiment, the biomass and/or further biomass comprises a bioactive. In an embodiment, the biomass and/or further biomass comprises a bioactive precursor.

In an embodiment, the bioactive and/or bioactive precursor is added to the biomass or further biomass.

In an embodiment, the biomass and/or further biomass is eukaryotic. In an embodiment, the biomass and/or further biomass is prokaryotic (e.g. algae). In an embodiment, the biomass and/or further biomass is from the Plantae or Fungi Kingdom.

The material may be any part of a Plantae or Fungi, including where relevant, but not limited to, one or more of leaves, stems, flowers, florets, seeds and roots.

In an embodiment, the Plantae is a Brassicaceae. As used herein, “Brassicaceae” refers to members of the Family Brassicaceae commonly referred to as mustards, crucifers or the cabbage family.

In an embodiment, the Brassicaceae is selected from the genus Brassica or Cardamine. In an embodiment, the Brassica is selected from one or more of: Brassica balearica, Brassica carinata, Brassica elongate, Brassica fruticulosa, Brassica hilarionis, Brassica juncea, Brassica napus (rapeseed or canola), Brassica narinosa, Brassica nigra, Brassica oleracea, Brassica perviridis, Brassica rapa, Brassica rupestris, Brassica septiceps, and Brassica tournefortii.

In an embodiment, the Brassica is Brassica oleracea.

In an embodiment, the Brassica is Brassica napus (rapeseed or canola).

In an embodiment, the Brassica selected from one or more of: Brassica oleracea variety oleracea (wild cabbage), Brassica oleracea variety capitate (cabbage), Brassica rapa subsp. chinensis (bok choy), Brassica rapa subsp. pekinensis (napa cabbage), Brassica napobrassica (rutabaga), Brassica rapa var. rapa (turnip), Brassica oleracea variety alboglabra (kai-lan), Brassica oleracea variety viridis (collard greens), Brassica oleracea variety longata (jersey cabbage), Brassica oleracea variety acephala (ornamental kale), Brassica oleracea variety sabellica (kale), Brassica oleracea variety palmifolia (lacinato kale), Brassica oleracea variety ramose (perpetual kale), Brassica oleracea variety medullosa (marrow cabbage), Brassica oleracea variety costata (tronchuda kale), Brassica oleracea variety gemmifera (brussels sprout), Brassica oleracea variety gongylodes (kohlrabi), Brassica oleracea variety italica (broccoli), Brassica oleracea variety botrytis (cauliflower, Romanesco broccoli, broccoli di torbole), Brassica oleracea variety botrytis x italica (broccoflower), and Brassica oleracea variety italica x alboglabra (Broccolini). In an embodiment, the Brassica oleracea is kale.

In an embodiment, the Brassica is Brassica oleracea, variety italica (broccoli). In an embodiment, the Brassica is Brassica oleracea variety botrytis (cauliflower).

In an embodiment, the Brassica is Brassica oleracea variety gemmifera (brussels sprout).

In an embodiment, the Brassicaceae is selected from one or more of: Cardamine hirsuta (bittercress), Iberis sempervirens (candytuft), Sinapis arvensis (charlock), Armoracia rusticana (horseradish), Pringlea antiscorbutica (kerguelen cabbage), Thlaspi arvense (pennycress), Raphanus raphanistrum subsp. sativus (radish), Eruca sativa (rocket), Anastatica hierochuntica (rose of jericho), Crambe maritima (sea kale), Cakile maritima (sea rocket), Capsella bursa-pastoris (shepherd's purse), sweet alyssum, Arabidopsis thaliana (thale cress), Nasturtium officinale (watercress), Sinapis alba (white mustard), Erophila verna (whitlow grass), Raphanus raphanistrum (wild radish), Isatis tinctoria (woad), and Nasturtium microphyllum (yellow cress).

In an embodiment, the biomass is Brassicaceae florets. In an embodiment, the biomass is Brassicaceae florets and stems. In an embodiment, the biomass is Brassicaceae stems and leaves.

In an embodiment, the Brassicaceae has a high level of one or more glucosinolate/s. In an embodiment, the Brassicaceae has been selectively bred to have a high level of one or more glucosinolate/s. In an embodiment, “high level” of a glucosinolate can comprise a higher level of a glucosinolate than shown in Table 2 of Verkerk et al. (2009) in the corresponding Brassicaceae. In an embodiment, a high level of glucosinolate is a level of glucosinolate higher than 3400 mol/kg dry weight. In an embodiment, a high level of glucosinolate is a level of glucosinolate higher than 4000 μmol/kg dry weight. In an embodiment, a high level of glucosinolate is a level of glucosinolate higher than 5000 μmol/kg dry weight. In an embodiment, a high level of glucosinolate is a level of glucosinolate higher than 8000 μmol/kg dry weight. In an embodiment, a high level of glucosinolate is a level of glucosinolate higher than 10,000 μmol/kg dry weight. In an embodiment, a high level of glucosinolate is a level of glucosinolate higher than 12,000 μmol/kg dry weight. In an embodiment, a high level of glucosinolate is a level of glucosinolate higher than 15,000 μmol/kg dry weight. In an embodiment, a high level of glucosinolate is a level of glucosinolate higher than 18,000 μmol/kg dry weight. In an embodiment, a high level of glucosinolate is a level of glucosinolate higher than 20,000 μmol/kg dry weight. In an embodiment, a high level of glucosinolate is a level of glucosinolate higher than 25,000 μmol/kg dry weight. In an embodiment, a high level of glucosinolate is a level of glucosinolate higher than 30,000 μmol/kg dry weight. In an embodiment, the Brassicaceae has been genetically modified or subjected to biotic or abiotic stress to have a high level of one or more glucosinolate/s. A person skilled in the art will appreciate that the Brassicaceae can be modified by any method known to a person skilled in the art.

In an embodiment, the glucosinolate is glucoraphanin (4-Methylsulphinylbutyl glucosinolate). In an embodiment, the glucosinolate is glucobrassicin (3-Indolylmethyl glucosinolate).

When the biomass is Brassicaceae, the biomass can be any part of the Brassicaceae, including, but not limited to, the leaves, stems, flowers, florets, seeds, and roots or mixtures thereof.

In an embodiment, the Plantae is Cannabis. In an embodiment, the Cannabis is Cannabis sativa (hemp).

In an embodiment, the Plantae is a fruit or vegetable. In an embodiment, the fruit is selected from one or more of: a simple, aggregate and multiple fruit. In an embodiment, the fruit or vegetable is from the family Brassicaceae, Musaceae, Convolvulaceae, Umbelliferae, Asparagaceae, Arecaceae, Myrtaceae, Rosaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae and Caesalpinioideae.

In an embodiment, Musaceae is a Musa. In an embodiment, the Musa is a ripe Musa fruit. In an embodiment, the Musa is an unripe (green) Musa fruit. In an embodiment, Musa is a banana. Banana contains approximately 75% moisture and 25% solids. As the banana ripens starch present in green banana is converted into sugars, with the amounts dependent on the stage of ripening, the banana variety and growing conditions. The starch content decreases from about 70% in unripe banana to about 1% in the ripe banana. In an embodiment, ripe banana comprises about 0.5% to about 3% starch. In an embodiment, banana comprises about 1% starch. In In an embodiment, Musa is a green banana. In an embodiment, green banana comprises about 70% to about 80% % starch. In an embodiment, green banana comprises about 75% % starch. Bananas may be converted to flours to increase shelf life. Banana flour made from bananas at ripening stage 1-2 contains 60-62% total starch, of which about 11-13% is resistant starch while banana flour made from bananas at ripening stage 3-4 contains about 96-97% starch of which about 37-38% is resistant starch. In an embodiment, the green banana is green banana flour.

In an embodiment, Convolvulaceae is an Ipomoea. In an embodiment, Ipomoea is a sweet potato.

In an embodiment, the Umbelliferae is carrot.

In an embodiment, the Asparagaceae is asparagus.

In an embodiment, the Polygonaceae is selected from one or more of: buckwheat, garden sorrel and rhubarb.

In an embodiment, the Cucurbitaceae is selected from one or more of: cucumber, pumpkin, squash and zucchini.

In an embodiment, the fruit is selected from one or more of: banana, green banana, apple, apricot, avocado, bilberry, blackberry, blackcurrant, blueberry, coconut, currant, cherry, cherimoya, clementine, cloudberry, damson, durian, elderberry, fig, feijoa, gooseberry, grape, grapefruit, guava, huckleberry, jackfruit, jambul, jujube, kiwifruit, kumquat, lemon, lime, loquat, lychee, mandarin, mango, melon, cantaloupe, honeydew, watermelon, nectarine, orange, passionfruit, paw paw, peach, pear, plum, plumcot, pineapple, pomegranate, pomelo, purple mangosteen, raspberry, rambutan, redcurrant, satsuma, star fruit, strawberry, tangerine, tomato, and ugli fruit. In an embodiment, the fruit is a banana. In an embodiment, the fruit is a green banana.

In an embodiment, the Plantae is a Compositae. In an embodiment, the Compositae is selected from one or more of: artichoke, chamomile, chicory, dandelion, endive, jerusalem artichoke, lettuce, romaine, safflower salsify and sunflower.

In an embodiment, the Plantae is an Amaranthaceae/Chenopodiacae. In an embodiment, the Amaranthaceae/Chenopodiacae is selected from one or more of: amaranth, beet, chard, lamb's-quarters, quinoa, spinach and sugar beet.

In an embodiment, the Plantae is Malvaceae. In an embodiment, the Malvaceae is selected from one or more of: cacao, cotton and okra.

In an embodiment, the Plantae is from the family Amarylidaceae. In an embodiment, the Amarylidaceae is from the subfamily Allioideae. In an embodiment, Allioideae is from the genus Allium. In an embodiment, the Allium is selected from one or more of: Allium sativum (garlic), Allium cepa (onion), Allium ampeloprasum (leeks), Allium schoenoprasum (chives), and Allium oschaninii (shallot).

In an embodiment, the Allium is Allium sativum (garlic).

In an embodiment, the Plantae is from the family Fabaceae. In an embodiment, the Fabaceae is soybean alfalfa, beans, carob, chickpea, green beans, jicama, lentil, pea, snow pea and peanut.

In an embodiment, the Fabaceae is snow pea.

In an embodiment, the Plantae is a cereal. In an embodiment, the cereal is an ancient grain. In an embodiment, the cereal is selected from one or more of: rice, corn, wheat, triticale, barley, millet, sorghum, spelt, oats, freekeh, bulgur, sorghum, farro, einkorn, teff, emmer and/or buckwheat.

In an embodiment, the Plantae is from the Arecaceae family. In an embodiment, the Arecaceae is the coconut palm. In an embodiment, the biomass and/or further biomass is the coconut drupe.

In an embodiment, the Plantae is a grass. In an embodiment, the grass is from the family Poaceae. In an embodiment, the grass is selected from one or more of: bamboo, lemongrass, sugarcane, corn and wheatgrass.

In an embodiment, the Plantae is from the family Camellia sinensis. In an embodiment, the Camellia sinensis is green tea leaves (matcha).

In an embodiment, the Fungi is a mushroom. In an embodiment, the Fungi is from the family Boletaceae, Cantharellaceae, Tricholomataceae, Cortinariaceae, Cantharellaceae, Meripilaceae, Discinaceae, Pleurotaceae, Tricholomataceae and Tuberaceae.

In an embodiment, the Fungi is selected from one or more of: Boletus edulis, Cantharellus cibarius, Cantharellus tubaeformis, Clitocybe nuda, Cortinarius caperatus, Craterellus cornucopioides, Grifola frondosa, Hericium erinaceus, Hydnum repandum, Lactarius deliciosus, Morchella conica var. deliciosa, Morchella esculenta var. rotunda, Pleurotus ostreatus, Tricholoma matsutake, Tuber brumale, Tuber indicum, Tuber macrosporum, Tuber mesentericum, and Tuber aestivum.

In an embodiment, the biomass and/or further biomass is not animal biomass or an animal produced product. In an embodiment, the biomass and/or further biomass is not avian. In an embodiment, the biomass and/or further biomass is not bone or bone marrow. In an embodiment, the biomass and/or further biomass is not animal milk.

In an embodiment, the biomass and/or further biomass is not milk, skim milk or purified milk protein and carbohydrate.

In an embodiment, the biomass and/or further biomass is not-fermented.

In an embodiment, the biomass and/or further biomass is not a ripe fruit of Musaceae.

In an embodiment, the biomass and/or further biomass is Plantae or Fungi material that does not meet cosmetic retail standards or is no longer suitable for fresh sale but still edible.

A person skilled in the art will appreciate that the methods as described herein are suitable for use with different volumes of biomass, for example, but not limited to, at least 30 kg, or at least 50 kg, or at least 80 kg, or at least 100 kg, or at least 1,000 kg, or at least 2,000 kg, or at least 5,000 kg, or at least 8,000 kg, or at least 10,000 kg, or at least 15,000 kg, or at least 20,000 kg.

### Washing/Sanitizing

In an embodiment, the biomass has been washed. As used herein “washing” removes visible soil and contamination. In an embodiment, the biomass has been sanitized. As used herein “sanitized” refers to a reduction of pathogens on the biomass. This, may include sterilization or partial sterilization.

### Pre-Treatment

In an embodiment, the biomass and/or further biomass as described herein is pre-treated. As used herein “pre-treatment” or “pre-treating” or “pre-treated” refers to processing of the biomass and/or further biomass to break the material into smaller components, remove a component (e.g. remove a specific component not suitable for ingestion or extract a specific component for a different use e.g. oil) or modify a component of the biomass and/or further biomass. In an embodiment, modifying a component includes, for example, producing a bioactive, or producing an oligosaccharide or a polysaccharide. In an embodiment, pre-treating does not alter the ratio of protein to carbohydrate in the biomass or further biomass. In an embodiment, pre-treating as described herein does not include fermentation (is non-fermented). As used herein “non-fermented” biomass is a biomass that has not been pre-treated with one or more bacteria (e.g. a lactic acid bacteria) to facilitate the breakdown of the biomass into one or more products (e.g. alcohol or lactic acid).

In an embodiment, pre-treating comprises one or more of the following: i) heating; ii) macerating; iii) microwaving; iv) exposure to low frequency sound waves (ultrasound); v) pulse electric field processing; vi) static high pressure; vii) extrusion; viii) enzyme treatment; ix) an extraction or separation process; and x) drying.

In an embodiment, the biomass and/or further biomass is heated in a fuel based heating system, an electricity based heating system (i.e. an oven or ohmic heating), radio frequency heating, high pressure thermal processing, ultra high temperature (UHT) treatment plant, in a retort or a steam based heating system (indirect or direct application of steam). In an embodiment, the biomass and/or further biomass is heated in an oven, water bath, bioreactor, stove, water blancher, or steam blancher. In an embodiment, the biomass and/or further biomass is heated via high pressure thermal heating. In an embodiment, the biomass and/or further biomass is heated via ohmic heating. In an embodiment, the biomass and/or further biomass is heated via radio frequency heating. In an embodiment, the biomass and/or further biomass is heated via high pressure thermal processing. In an embodiment, the biomass and/or further biomass is placed in a sealed pack or container for high pressure thermal processing.

In an embodiment, pre-treating comprises heating the biomass and/or further biomass to about 50° C. to about 140° C. In an embodiment, heating comprises heating the biomass and/or further biomass to about 55° C. to about 70° C. In an embodiment, heating comprises heating the biomass and/or further biomass to about 60° C. to about 70° C. In an embodiment, heating comprises heating the biomass and/or further biomass to about 65° C. to about 70° C. In an embodiment, heating comprises heating the biomass and/or further biomass to about 70° C. to about 140° C. In an embodiment, heating comprises heating the biomass and/or further biomass to about 80° C. to about 130° C. In an embodiment, heating comprises heating the biomass and/or further biomass to about 90° C. to about 120° C. In an embodiment, heating comprises heating the biomass and/or further biomass to about 100° C. to about 110° C. In an embodiment, heating comprises heating the biomass and/or further biomass to about 75° C. for about 2 minutes. In an embodiment, heating comprises heating the biomass and/or further biomass to about 100° C. for about 30 minutes. In an embodiment, pre-treatment comprises heating at the lower end of the above temperature range for a longer period of time or treatment at the higher end of the above temperature range for a short period of time.

In an embodiment, heating comprises steaming the biomass and/or further biomass. In an embodiment, the biomass and/or further biomass is steamed to a temperature of about 100° C. In an embodiment, the biomass and/or further biomass is steamed for at least about 30 seconds. In an embodiment, the biomass and/or further biomass is steamed for at least 1 minute. In an embodiment, the biomass and/or further biomass is steamed for at least 2 minutes. In an embodiment, the biomass and/or further biomass is steamed for at least 3 minutes. In an embodiment, the biomass and/or further biomass is steamed for at least 4 minutes. In an embodiment, the biomass and/or further biomass is steamed for at least 5 minutes. In an embodiment, the biomass and/or further biomass is steamed to a temperature of about 100° C. for 30 minutes.

In an embodiment, heating comprises ultra high temperature (UHT) treatment of the biomass and/or further biomass. In an embodiment, the biomass and/or further biomass is UHT treated at a temperature of about 140° C.

In an embodiment, heating comprises retorting of the biomass and/or further biomass. In an embodiment, the biomass and/or further biomass is retorted at a temperature of about 116° C. to about 130° C.

In an embodiment, pre-treating comprises macerating the biomass and/or further biomass. In an embodiment, the biomass and/or further biomass is macerated with a shredder, blender, colloid mill, grinder or pulveriser. In an embodiment, the biomass and/or further biomass is macerated so that at least 80% of the biomass and/or further biomass is of a size of 2 mm or less. In an embodiment, the biomass and/or further biomass is macerated so that at least 80% of the biomass and/or further biomass is of a size of 1 mm or less. In an embodiment, the biomass and/or further biomass is macerated so that at least 80% of the biomass and/or further biomass is of a size of 0.5 mm or less. In an embodiment, the biomass and/or further biomass is macerated so that at least 80% of the biomass and/or further biomass is of a size of 0.25 mm or less. In an embodiment, the biomass and/or further biomass is macerated so that at least 80% of the biomass and/or further biomass is of a size of 0.1 mm or less. In an embodiment, the biomass and/or further biomass is macerated so that at least 80% of the biomass and/or further biomass is of a size of 0.05 mm or less. In an embodiment, the biomass and/or further biomass is macerated so that at least 80% of the biomass and/or further biomass is of a size of 0.025 mm or less. In an embodiment, the biomass and/or further biomass is heated during maceration. In some embodiments, heating facilitates the conversion of bioactive precursors into a bioactive, such as for example, sulforaphane and ajoene. In an embodiment, the biomass and/or further biomass is heated to a temperature of about 25° C. to about 80° C. during maceration. In an embodiment, the biomass and/or further biomass is heated to a temperature of about 40° C. to about 70° C. during maceration. In an embodiment, the biomass and/or further biomass is heated to a temperature of about 50° C. to about 70° C. during maceration. In an embodiment, the biomass and/or further biomass is heated to a temperature of about 60° C. to about 70° C. during maceration. In an embodiment, the biomass and/or further biomass is heated to a temperature of about 70° C. during maceration for about 2 to about 5 mins. In an embodiment, the biomass and/or further biomass is heated to a temperature of about 30° C. to about 80° C. during maceration for about 1 to about 5 hours.

In an embodiment, pre-treating comprises heating and macerating the biomass and/or further biomass.

A person skilled in the art will appreciate that “microwaves” or “microwaving” heats a substance such as biomass and/or further biomass by passing microwave radiation through the substance. In an embodiment, pre-treating comprises microwaving the biomass and/or further biomass. In an embodiment, biomass and/or further biomass is pre-treated in a consumer microwave or industrial microwave. In an embodiment, the industrial microwave is a continuous microwave system, for example, but not limited to the MIP 11 Industrial Microwave Continuous Cooking Over (Ferrite Microwave Technologies). In an embodiment, pre-treating comprises microwaving the biomass and/or further biomass. In an embodiment, the biomass and/or further biomass is microwaved at about 0.9 to about 2.45 GHz. In an embodiment, the biomass and/or further biomass is microwaved for at least 30 seconds, or at least 1 minute, or at least 2 minutes, or at least 3 minutes. In an embodiment, microwaving increases the temperature of the biomass and/or further biomass to about 70 to about 80° C., preferably about 76° C.

In an embodiment, pre-treating comprises exposing the biomass and/or further biomass at low to medium frequency ultrasound waves. In an embodiment, pre-treating comprises exposing the biomass and/or further biomass to thermosonication (low to medium frequency ultrasound waves with heat of about 50° C. to about 140° C.). In an embodiment, the ultrasound waves are generated with an industrial scale ultrasonic processor. In an embodiment, the ultrasonic processor is a continuous or batch ultrasonic processor. In an embodiment, the ultrasonic processor is for example, but not limited to, UIP500hd or UIP4000 (Hielscher, Ultrasound Technology). In an embodiment, the ultrasounds waves are at a frequency of about 20 kHz to about 600 kHz. In an embodiment, the biomass and/or further biomass is exposed to sound waves for at least 30 seconds, or at least 1 minute, or at least 2 minutes, or at least 3 minutes, or about 5 minutes, or about 6 minutes, or about 7 minutes, or about 7.5 minutes, or about 8 minutes.

In an embodiment, pre-treating comprises exposing the biomass and/or further biomass to pulse electric field processing. Pulse electric field processing is a non-thermal processing technique comprising the application of short, high voltage pulses. The pulses induce electroporation of the cells of the biomass and/or further biomass. In an embodiment, pulse electric field processing heats the biomass and/or further biomass to a temperature of about 50 to about 140° C. In an embodiment, pulse electric field processing heats the biomass and/or further biomass to a temperature of about 70° C. to about 110° C. In an embodiment, pulse electric field processing heats the biomass and/or further biomass to a temperature of about 80° C. to about 100° C. In an embodiment, pulse electric field processing comprises treating the biomass and/or further biomass with voltage pulses of about 20 to about 80 kV.

In an embodiment, pre-treating comprises hydrostatic pressure. In an embodiment, hydrostatic pressure comprises treating the biomass and/or further biomass with about 100 to about 600 MPa.

In an embodiment, pre-treating comprises extrusion. In an embodiment, extrusion comprises applying a force to the biomass or product, usually at elevated temperature and/or high pressure through a barrel prior to expulsion of the mass through an orifice. In an embodiment, the high temperatures, high pressures and mechanical forces applied during extrusion modify the functional properties of the material. In an embodiment, the extrusion process is carried out using a co-rotating twin screw extruder (MPF 18:25, APV Baker Ltd., Peterborough, UK) or a lab-scale, co-rotating and intermeshed twin-screw lab extruder (KDT30-II, Jinan Kredit Machinery Co. Ltd., China). In an embodiment, the extrusion process produces Maillard reaction products.

In an embodiment, pre-treating comprises enzyme treatment to transform one or more components in the biomass and/or further biomass to a new component. For example, the enzyme converts simple sugars into oligosaccharides or polysaccharides. In an embodiment, the enzyme is selected from one or more of a: glycosyltransferase, ii) glycosidase, iii) pectinase, iv) esterase, v), oxidoreductase, vi) protease, vii) pectinase, viii) polygalacturonase, ix) amylase and x) pullulanase. In an embodiment, the glycosyltransferase is selected from one or more or all of a: i) dextransucrase, ii) alternansucrase, and iii) fructosyltransferases. In an embodiment, the fructosyltransferases is for example levansucrase, and/or inulosucrase. In an embodiment, the oxidoreductase is mannitol dehydrogenase.

In an embodiment, pre-treatment releases or aids in the release of a glucosinolate from glucosinolate storage site and/or allows myrosinase to enter a glucosinolate storage site in the biomass and/or further biomass. In an embodiment, pre-treating increases the exposure of a glucosinolate to myrosinase allowing myrosinase to convert a glucosinolate to an isothiocyanate.

In an embodiment, pre-treating converts about 10% to about 90% of a glucosinolate to an isothiocyanate. In an embodiment, pre-treating converts about 20% to about 80% of a glucosinolate to an isothiocyanate. In an embodiment, pre-treating converts about 30% to about 70% of a glucosinolate to an isothiocyanate. In an embodiment, pre-treating converts about 40% to about 60% of a glucosinolate to an isothiocyanate. In an embodiment, pre-treating converts about 10% of a glucosinolate to an isothiocyanate. In an embodiment, pre-treating converts about 20% of a glucosinolate to an isothiocyanate. In an embodiment, pre-treating converts about 30% of a glucosinolate to an isothiocyanate. In an embodiment, pre-treating converts about 40% of a glucosinolate to an isothiocyanate. In an embodiment, pre-treating converts about 50% of a glucosinolate to an isothiocyanate. In an embodiment, pre-treating converts about 60% of a glucosinolate to an isothiocyanate. In an embodiment, pre-treating converts about 70% of a glucosinolate to an isothiocyanate. In an embodiment, pre-treating converts about 80% of a glucosinolate to an isothiocyanate. In an embodiment, pre-treating converts about 90% of a glucosinolate to an isothiocyanate.

In an embodiment, pre-treating comprises treating the biomass and/or further biomass with an extraction or separation process to reduce the amount of one or more components in the biomass and/or further biomass (e.g. the biomass may be canola meal where the canola oil has been removed or partially removed). In an embodiment, the other components are suitable for producing other products or are non-edible or poor tasting components of the biomass and/or further biomass.

In an embodiment, the extraction or separation process is for the removal of a component selected from oil, a bioactive or bioactive precursor, polyphenols, carotenoids, or juice from the biomass. In an embodiment, the extraction or separation process produces canola meal, nut meal, soybean meal, coconut meal, palm kernel meal, hemp oil press cakes, chia oil seed cake or rice bran which may be used as a biomass in the methods as described herein. In an embodiment, the extraction or separation process produces pomace (e.g. olive or apple pomace) which may be used as a biomass in the methods as described herein. In an embodiment, the extraction or separation process may comprises removing a non-edible component from the biomass (e.g. seeds or stalks). In an embodiment, the extraction or separation process may comprise grinding, cutting, milling, centrifugation and/or filtration.

As used herein “reduced” means that the level of a component is lower in the biomass or further biomass after treatment with the extraction process than in the biomass or further biomass before treatment with the extraction process.

In an embodiment, the level of the component is reduced from about 5% to about 90%. In an embodiment, the level of the component is reduced by about 5%. In an embodiment, the level of the component is reduced by about 10%. In an embodiment, the level of the component is reduced by about 15%. In an embodiment, the level of the component is reduced by about 20%. In an embodiment, the level of the component is reduced by about 30%. In an embodiment, the level of the component is reduced by about 40%. In an embodiment, the level of the component is reduced by about 50%. In an embodiment, the level of the component is reduced by about 60%. In an embodiment, the level of the component is reduced by about 70%. In an embodiment, the level of the component is reduced by about 80%. In an embodiment, the level of the component is reduced by about 90%. In an embodiment, the level of the component is reduced by about 100%.

In an embodiment, pre-treating comprises drying or partially drying the biomass. In an embodiment, drying comprises tray drying, drum drying, roller drying, fluid bed drying, impingement drying, spray drying, freeze-drying (lyophilisation or cryodesiccation), thin-film belt dryer, vacuum microwave drying, ultrasonic-assisted drying, extrusion porosification technology or any other method known to a person skilled in the art. In an embodiment, pre-treating comprises freeze-drying the biomass. In an embodiment, pre-treating comprises heating then freeze-drying the biomass. In an embodiment, pre-treating comprises drum drying. In an embodiment pre-treating comprises spray drying.

A person skilled in the art would appreciate that pre-treating does not comprise separately purifying protein and purifying carbohydrate from the biomass.

In an embodiment, pre-treatment of the biomass increases the bioavailability of one or more components of the biomass. In an embodiment, the component is fibre. In an embodiment, the component is a prebiotic and/or prebiotic precursor. In an embodiment, the prebiotic is selected from one or more or all of: dietary fibre (insoluble/soluble), oligosaccharides, cellulose, hemicellulose, pecticoligosaccharide, resistant starch beta-glucans and pectin.

In an embodiment, the component is a polyphenol. As used herein, “polyphenol” refers to a compound comprising more than one phenolic hydroxyl group. In an embodiment, the polyphenol is selected from one or more of: anthocyanins, dihydrochalcones, flavan-3-ols, flavanones, flavones, flavonols and isoflavones, curcumin, resveratrol, benzoic acid, phenyl acetic acid, hydroxycinnamic acids, coumarins, napthoquinones, xanthones, stilbenes, chalcones, tannins, phenolic acids, and catechins (e.g. epigallocatechin gallate (EGCg), epigallocatechin (EGC), epicatechin gallate (ECg), epicatechin (EC), and their geometric isomers gallocatechin gallate (GCg), gallocatechin (GC), catechin gallate (Cg) and catechin.

### Lipid

In an embodiment, a method as described herein further comprises the addition of a lipid. As used herein “lipid” refers to a ester of a long straight-chain carboxylic acid that is insoluble in water but soluble in an organic solvent. In an embodiment, the lipid is saponifiable. In an embodiment, the lipid is an oil as described herein. In an embodiment, the lipid is a wax as described herein. In an embodiment, the lipid may be an oil where omega-3 fatty acids are bound to phospholipid (e.g. krill oil).

### Oils

In an embodiment, the composition, product or method as described herein further comprises an oil. As used herein “oil” refers to a viscous liquid that is hydrophobic and lipophilic and not miscible with water. In an embodiment, the oil is susceptible to deterioration by one or more of oxidation, temperature, pH, moisture, light and pro-oxidants.

In an embodiment, the oil comprises a fatty acid as described herein. As used herein, the term “fatty acid” refers to a carboxylic acid (or organic acid), often with a long aliphatic tail, either saturated or unsaturated. Typically fatty acids have a carbon-carbon bonded chain of at least 4 carbon atoms (C4) or at least 8 carbon atoms (C8) in length, more preferably at least 12 carbons in length. Preferred fatty acids of the invention have carbon chains of 18-22 carbon atoms (C18, C20, C22 fatty acids), more preferably 20-22 carbon atoms (C20, C22) and most preferably 22 carbon atoms (C22). Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms. In an embodiment, the fatty acid is esterified to a methyl or ethyl group, such as, for example, a methyl or ethyl ester of a C20 or C22 polyunsaturated fatty acid. Preferred fatty acids are the methyl or ethyl esters of eicosatrienoic acid, docosapentaenoic acid or docosahexaenoic acid, or the mixtures eicosapentaenoic acid and docosahexaenoic acid, or eicosapentaenoic acid, docosapentaenoic acid and docosahexaenoic acid, or eicosapentaenoic acid and docosapentaenoic acid.

In an embodiment, the fatty acid is a polyunsaturated fatty acid. As used herein “polyunsaturated fatty acid” refers to a fatty acid that contains more than one double bond in its backbone. In an embodiment, the polyunsaturated fatty acid is selected from one or more of: an omega-3, omega-6, or omega-9. In an embodiment, the polyunsaturated fatty acid is an omega-3. In an embodiment, the polyunsaturated fatty acid is an omega-6. In an embodiment, the polyunsaturated fatty acid is an omega-9. In an embodiment, the omega-3 is selected from one or more of: hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In an embodiment, the omega-3 is selected from one or more or all of eicosapentaenoic acid, docosapentaenoic acid and docosahexaenoic acid. In an embodiment, the omega-6 is selected from one or more of: linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, and tetracosapentaenoic acid. In an embodiment, the omega-9 is selected from one or more of: oleic acid, eicosenoic acid, mead acid, erucic acid, and nervonic acid.

In an embodiment, the oil is a Plantae oil. In an embodiment, the oil is a vegetable oil. In an embodiment, the oil is a nut oil. In an embodiment, the oil is an animal oil. In an embodiment, the animal oil is a marine oil or fish oil.

In an embodiment, the oil is selected from one or more of: fish oil, krill oil, algal oil, marine oil, fungal oil, nut or seed oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, medium chain triglycerides (MCT) and grapeseed oil. In an embodiment, the oil is algal oil. In an embodiment, the canola oil comprises one or more long chain polyunsaturated fatty acids such as eicosapentaenoic acid (EPA), docosapentaenoic acid (DPA) and docosahexaenoic acid (DHA) which can be obtained from transgenic Brassica encoding the required elongases and desaturases (see, for example, WO 2015/089587).

In an embodiment, the fish oil is selected from one or more of: tuna oil, herring oil, mackerel oil, anchovy oil, sardine oil, cod liver oil, and shark oil.

In an embodiment, the nut oil or seed oil is selected from one or more of: coconut oil, almond oil, avocado oil, canola oil, corn oil, hazelnut oil, palm oil peanut oil, pine seed oil, pumpkin seed oil, safflower oil, sesame oil, and walnut oil.

In an embodiment, the essential oil is selected from one or more of: oregano oil, mint oil, basil oil, rosemary oil, tea tree oil, time oil, camphor oil, cardamon oil, citrus oil, clove oil, and/or saffron oil. In an embodiment, the oil comprises dairy fats. In an embodiment, the oil is olive oil. In an embodiment, the oil is sunflower oil. In an embodiment, the oil is canola oil.

### Preparation of a Composition

In an aspect, the present invention provides a method of producing a powder composition, the method comprising

i) producing an aqueous mixture comprising
a) protein and carbohydrate from a non-fermented biomass from a single species of organism, and
b) a probiotic, and

ii) forming, from the mixture, a powder comprising the probiotic entrapped or encapsulated in a matrix comprising the protein and the carbohydrate.

In an embodiment, the method does not comprise fermentation.

In an embodiment, the entrapped or encapsulated probiotic has higher viability compared to the unentrapped or unencapsulated probiotic.

In an embodiment, the composition is synbiotic.

In an embodiment, the probiotic is added to the biomass before, during or after producing an aqueous mixture. In an embodiment, the probiotic is added to the biomass before producing aqueous mixture. In an embodiment, the probiotic is added to the biomass during the process of producing an aqueous mixture. In an embodiment, the probiotic is added to the biomass after an aqueous mixture is produced.

In an embodiment, the method comprises forming an emulsion or suspension.

As used herein “emulsion” refers to a dispersion of droplets/particles of one liquid in another in which it is not soluble or miscible. In one embodiment, the droplets are fatty acid and/or oil dispersed in the aqueous mixture. In an embodiment, the emulsion is a wet emulsion. In an embodiment, the emulsion is dried into powder. In an embodiment, the emulsion is extruded. In an embodiment, the emulsion is extruded with a powder matrix.

In an embodiment, droplets produced by the methods described herein are about 0.2 μm to about 10 μm. In an embodiment, droplets produced by the methods described herein are about 1 μm to about 10 μm. In an embodiment, droplets produced by the methods described herein are about 2 μm to about 8 μm. In an embodiment, droplets produced by the methods described herein are about 2 μm to about 4 μm.

In an embodiment, the mean droplet size is about 0.2 μm to about 10 μm. In an embodiment, the mean droplet size is about 1 μm to about 10 μm. In an embodiment, the mean droplet size is about 2 μm to about 8 μm. In an embodiment, the mean droplet size is about 2 μm to about 4 μm.

As used herein “suspension” refers to dispersion of droplets/particles of one substance throughout the bulk of another substance. In one embodiment, the droplets are a fatty acid and/or oil dispersed in the aqueous mixture.

As used herein forming an emulsion or suspension refers to entrapment or encapsulation of a substance in the aqueous mixture reducing the exposure of the substance to degradation. In an embodiment, the substance is a fatty acid and/or oil.

In an embodiment, the fatty acid and/or oil is heated when it is added to the aqueous mixture in step ii) as described herein. In an embodiment, the fatty acid and/or oil is heated to about 30° C. to about 80° C. In an embodiment, the fatty acid and/or oil is heated to about 40° C. to about 70° C. In an embodiment, the fatty acid and/or oil is heated to about 45° C. to about 65° C. In an embodiment, the fatty acid and/or oil is heated to about 50° C. to about 60° C.

In an embodiment, forming an emulsion or suspension as described herein comprises mixing of the fatty acid and/oil with an aqueous mixture comprising the biomass.

In an embodiment, mixing comprises agitation under high shear. In an embodiment, mixing comprises homogenization to obtain a small droplet size. In an embodiment, droplets produced by homogenization are about 0.2 μm to about 10 μm in diameter. In an embodiment, droplets produced by homogenization are about 1 μm to about 10 μm in diameter. In an embodiment, droplets produced by homogenization are about 2 μm to about 8 μm in diameter. In an embodiment, droplets produced by homogenization are about 2 μm to about 4 μm in diameter. In an embodiment, homogenization forms a homogenous emulsion.

In an embodiment, the method further comprises adding oil to the aqueous mixture and forming an emulsion or a suspension. In an embodiment, the probiotic is added to the oil prior to, during formation of or after formation of the emulsion or suspension. In an embodiment, the probiotic is added to the oil prior to formation of the emulsion or suspension. In an embodiment, the probiotic is added during formation of the emulsion or suspension. In an embodiment, the probiotic is added after formation of the emulsion or suspension.

In an embodiment, the oil is entrapped or encapsulated in the matrix, and wherein the entrapped or encapsulated oil is resistant to degradation compared to the unentrapped or unencapsulated oil. In an embodiment, the oil is resistant to degradation by one or more of: oxygen, temperature, pH, moisture, light, and pro-oxidants.

As used herein “resistant to degradation” refers to resistance to degradation of an oil due to exposure to an environmental factor that can cause degradation. In an embodiment, the sensitivity to an environmental factor is reduced by binding of the oil to a protein or carbohydrate in the biomass as described herein.

As used herein, the term “resistant to oxygen degradation”, “resistant to degradation by oxygen” or similar phrases, refers to reducing the susceptibility of an oil to oxidation. In an embodiment, the susceptibility of the oil to oxidation is reduced by entrapping or encapsulating the substance to reduce exposure to oxygen. In an embodiment, this includes entrapping or encapsulating the substance with molecules with oxygen sequestration ability. Assessment of oxidative resistance may be performed by any method known to a person skilled in the art. For example, the oxidative resistance of an oil may be based on the oxidation of oil with oxygen under pressure. In such a test, the consumption of oxygen, results in a pressure drop during the test which is due to the uptake of oxygen by the sample during oxidation. The oxidation rate is accelerated when carried out at elevated pressure and temperature. In an embodiment, the oxidative resistance is assessed using an Oxipres (e.g. a Mikrolab Aarhus A/S apparatus Højbjerg, Denmark). In an embodiment, a composition containing an oil (e.g. polyunsaturated oils) is exposed to high temperature and high oxygen pressure. In an embodiment, the oxidative resistance is assessed at 80° C. and 5 bar initial oxygen pressure. In an embodiment, the induction period (IP, h) is determined, which is related to oxidative stability of the samples. A longer IP (h) indicates that a sample is more resistant (more stable in the presence of oxygen) to oxidation during storage. Other methods for measuring oxidation include, for example, peroxide value, para-anisidine value and headspace analysis of volatiles (e.g. aldehydes such as propanal and EE-2,4-heptadienal which are secondary oxidation products from oxidation of omega-3 fatty acids) and change in % individual unsaturated fatty acids (e.g. EPA and DHA) in stored samples. In an embodiment, oxidation is not necessarily relative to solvent extractable free-fat (i.e. the free fat level is not an indicator of IP or susceptibility of oils to oxidation in powders). As used herein “resistant to temperature degradation”, “resistant to degradation by temperature” or similar phrases, refers to degradation of an oil due to exposure to low or high temperature. In an embodiment, the sensitivity to degradation by temperature is reduced by binding of the oil to a protein or carbohydrate in the biomass as described herein. In an embodiment, improved resistance to temperature degradation can be assessed under accelerated conditions by storing a sample at about 40° C. for about 1 week to about 2 weeks and comparing degradation to a control sample.

As used herein “resistant to moisture degradation”, “resistant to degradation by moisture” or similar phrases, refers to degradation of an oil due to exposure to low or high moisture. In an embodiment, the sensitivity to degradation by moisture is reduced by binding of the oil to a protein or carbohydrate in the biomass as described herein.

As used herein, the term “resistant to pH degradation”, “resistant to degradation by pH” or similar phrases, refers to the degradation of an oil due to exposure to a low or high pH. In an embodiment, low pH is a pH <7. In an embodiment, high pH is a pH >7. In an embodiment, the sensitivity to degradation by pH is reduced by binding of the oil to a protein or carbohydrate in the biomass as described herein.

As used herein, the term “resistant to light degradation”, “resistant to degradation by light” or similar phrases, refers to the degradation of an oil due to exposure to light. In an embodiment, the sensitivity to light degradation is reduced by binding of the oil to a protein or carbohydrate in the biomass as described herein.

As used herein, the term “resistant to pro-oxidants”, “resistant to degradation by pro-oxidants” or similar phrases, refers to the degradation of an oil by chemicals e.g. metal ions of vitamins that induce oxidative stress either by generating reactive oxygen species or by inhibiting antioxidant systems. In an embodiment, the sensitivity to degradation by pro-oxidants is reduced by binding of the oil to a protein or carbohydrate in the biomass as described herein. In an embodiment, the oil is resistant to degradation for at least two weeks when stored at 40° C.

In an embodiment, the method further comprises adding protein and carbohydrate from at least one further biomass from a single species of organism.

In an embodiment, the biomass or further biomass is from the Plantae or Fungi Kingdom. In an embodiment, the Plantae is selected from: Brassicaceae, Cannabis Asparagaceae, Arecaceae, Myrtaceae, Rosaceae, Musaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae, Caesalpinioideae, Compositae, Amaranthaceae, Chenopodiacae, Malvaceae, Amarylidaceae, Fabaceae, Arecaceae and Poaceae. In an embodiment, the Plantae is Brassicaceae. In an embodiment, the Brassicaceae is broccoli. In an embodiment, the method further comprising pre-treating the biomass as described herein.

In an embodiment, the aqueous mixture, emulsion or suspension as described herein is partially dried or dried to reduce the water content. In an embodiment, the method as described herein comprises drying the aqueous mixture, emulsion or suspension to reduce the water content to about 1 to about 14%. In an embodiment, the method as described herein comprises drying the aqueous mixture, emulsion or suspension to reduce the water content to about 1 to about 13%. In an embodiment, the method comprises drying the aqueous mixture, emulsion or suspension to reduce the water content to about 1 to about 12%. In an embodiment, the method comprises drying the aqueous mixture, emulsion or suspension to reduce the water content to about 1 to about 10%. In an embodiment, the method comprises drying the aqueous mixture, emulsion or suspension to reduce the water content to about 2 to about 8%. In an embodiment, the method comprises drying the aqueous mixture, emulsion or suspension to reduce the water content to about 2 to about 6%. In an embodiment, the method comprises drying the aqueous mixture, emulsion or suspension to reduce the water content to about 2 to about 4%. In an embodiment, the method comprises drying the aqueous mixture, emulsion or suspension to reduce the water content to about 2 to about 3%.

In an embodiment, the method as described herein comprises drying the aqueous mixture, emulsion or suspension to reduce the water activity to a low water activity to about 0.1 to about 0.7. In an embodiment, the method comprises drying the aqueous mixture, emulsion or suspension to reduce the water activity to a low water activity to about 0.2 to about 0.6. In an embodiment, the method comprises drying the aqueous mixture, emulsion or suspension to reduce the water activity to a low water activity to about 0.2 to about 0.5. In an embodiment, the method comprises drying the aqueous mixture, emulsion or suspension to reduce the water activity to a low water activity to about 0.3 to about 0.4. In an embodiment, the method comprises drying the aqueous mixture, emulsion or suspension to reduce the water activity to a low water activity of about 0.4

In an embodiment, the method as described herein comprises drying the aqueous mixture, emulsion or suspension to form a powder. Drying may include for example spray drying, freeze-drying (lyophilisation or cryodesiccation), tray drying, drum drying, roller drying, fluid bed drying, impingement drying, refractance windows drying, thin-film belt drying, vacuum microwave drying, ultrasonic-assisted drying, extrusion porosification technology or any other method known to a person skilled in the art.

In an embodiment, the aqueous mixture, emulsion or suspension is dried to produce a mean dry particle size of about 10 μM to about 4000 μM. In an embodiment, the aqueous mixture, emulsion or suspension is dried to produce a mean dry particle size of about 10 μM to about 3000 μM. In an embodiment, the aqueous mixture, emulsion or suspension is dried to produce a mean dry particle size of about 20 μM to about 2000 μM. In an embodiment, the aqueous mixture, emulsion or suspension is dried to produce a mean dry particle size of about 10 μM to about 1000 μM. In an embodiment, the aqueous mixture, emulsion or suspension is dried to produce a mean dry particle size of about 10 μM to about 500 μM.

In an embodiment, the aqueous mixture, emulsion or suspension is dried by spray drying (e.g. a Drytec laboratory spray dryer) to form a powder. For example, the aqueous mixture, emulsion or suspension is dried using a Drytec laboratory spray dryer with a rotary atomiser, ultrasonic nozzle or twin fluid nozzle at 2.0-4.0 bar atomising pressure by heating the feed to 60° C. prior to atomisation and the inlet and outlet air temperatures were 180° C. and 80° C., respectively. In an embodiment, the spray dryer has a granulation function. In an embodiment, the spray dryer is mounted with a granulation dryer. In an embodiment, spray drying produces individual particles or agglomerates of particles.

In an embodiment, spray drying produces a mean dry particle size of about 10 μM to about 3000 μM. In an embodiment, spray drying produces a mean dry particle size of about 20 μM to about 2000 μM. In an embodiment, spray drying produces a mean dry particle size of about 10 μM to about 1000 μM. In an embodiment, spray drying produces a mean dry particle size of about 10 μM to about 500 μM.

In an embodiment, the aqueous mixture, emulsion or suspension is dried by freeze-drying to form a powder. In an embodiment, a cryoprotectant is added to the aqueous mixture, emulsion or suspension before freeze drying. In an embodiment, the cryoprotectant is a monosaccharide, disaccharide or polysaccharide, polyalcohol or a derivative thereof. In an embodiment, the cryoprotectant is selected from one or more of: trehalose, sucrose and mannitol.

In an embodiment, the aqueous mixture, emulsion or suspension is dried by drum drying to form a powder.

In an embodiment, the powder comprises about 5% to about 50% oil w/w. In an embodiment, the powder about 10% to about 50% oil w/w. In an embodiment, the powder comprises about 20% to about 50% oil w/w. In an embodiment, the powder comprises about 20% to about 50% oil w/w. In an embodiment, the powder comprises about 20% to about 40% oil w/v. In an embodiment, the powder comprises about 20% to about 30% oil w/w.

In an embodiment, the powder comprises particles of about 20 μm to about 1200 μm. In an embodiment, the powder comprises particles of about 100 μm to about 900 μm. In an embodiment, the powder comprises particles of about 400 μm to about 700 μm. In an embodiment, the powder comprises particles of about 500 μm to about 600 μm. In an embodiment, the powder comprises particles of about 1000 μm. In an embodiment, the powder is milled to further reduce the particle size. In an embodiment, milling may reduce the particle size to less than about 10 μm, or less than about 8 μm, or less than about 6 μm, or less than about 4 μm, or less than about 2 μm.

In an embodiment, the oil entrapped or encapsulated in a powder by the methods described herein is less susceptible (more resistant) to oxygen degradation than the oil entrapped or encapsulated by the MicroMAX® encapsulation method (WO01/74175).

### Acidification/pH Adjustment

The pre-treated material can be acidified to improve the microbial safety and stability (susceptibility to microbial degradation) of the product. Acidification can be achieved by the addition of organic acids, such as, but not limited to lactic, acetic, ascorbic, and citric acid. In embodiment, acidification can be achieved with the addition of glucono-delta-lactone. In an embodiment, acidification comprises lowering the pH to a pH of about 4.4 to about 3.4. In an embodiment, acidification comprises lowering the pH to a pH of 4.5, or 4.4, or 4.2, or 4, or 3.8, or 3.6, or 3.4 or less. In an embodiment, acidification comprises lowering the pH to a pH of 4.4 of less.

The pH of the composition or product as described herein can be adjusted to alter the activity of the composition or product when it is administered to a subject. In an embodiment, the composition or product as described herein is adjusted to a pH of about 4.5 to about 7.5. In an embodiment, the composition or product as described herein is adjusted to a pH of about 5 to about 7. In an embodiment, the composition or product as described herein is adjusted to a pH of about 5.5 to about 6.8. In an embodiment, the composition or product as described herein is adjusted to a pH of about 6 to about 6.8. In an embodiment, the composition or product as described herein is adjusted to a pH of about 6.2 to about 6.8. In an embodiment, the composition or product as described herein is adjusted to a pH of about 6.4 to about 6.8. In an embodiment, the composition or product as described herein is adjusted to a pH of about 6.8.

### Compositions and Products

In an aspect, the present invention provides a powder composition comprising a probiotic entrapped or encapsulated in a matrix comprising protein and carbohydrate from a non-fermented biomass from a single species of organism.

In an embodiment, the entrapped or encapsulated probiotic has higher viability compared to the unentrapped or unencapsulated probiotic. In an embodiment, the composition comprises a prebiotic and a probiotic. In an embodiment, the composition is synbiotic.

In an embodiment, the composition is dairy free. In an embodiment, the composition is animal free.

In an embodiment, the composition is vegan.

In an embodiment, the composition comprises at least one serving of vegetable. In an embodiment, the composition comprises at least two servings of vegetable. In an embodiment, one serving of vegetable is about 50 to about 100 g of vegetable. In an embodiment, one serving of vegetable is about 75 g of vegetable. In an embodiment, one serving of vegetable is about 100 g of vegetable. In an embodiment, one serving of vegetable comprises about 100 to about 350 kJ. In an embodiment, one serving of vegetable comprises about 150 to about 300 kJ. In an embodiment, one serving of vegetable comprises about 200 to about 250 kJ.

In an embodiment, the composition comprises at least one serving of fruit. In an embodiment, the composition comprises at least two servings of fruit. In an embodiment, one serving of fruit is about 50 to about 100 g of fruit. In an embodiment, one serving of fruit is about 75 g of fruit. In an embodiment, one serving of fruit is about 100 g of fruit. In an embodiment, one serving of fruit comprises about 100 to about 350 kJ. In an embodiment, one serving of fruit comprises about 150 to about 300 kJ. In an embodiment, one serving of fruit comprises about 200 to about 250 kJ

In an embodiment, the composition comprises at least one serving of omega-3. In an embodiment, one serving of omega-3 comprises about 200 to about 750 mg omega-3. In an embodiment, one serving of omega-3 comprises about 250 to about 600 mg omega-3. In an embodiment, one serving of omega-3 comprises about 250 to about 500 mg omega-3. In an embodiment, one serving of omega-3 comprises about 300 to about 400 mg omega-3. In an embodiment, one serving of omega-3 comprises about 300 mg omega-3.

In an embodiment, the composition comprises at least one serving of omega-6. In an embodiment, one serving of omega-6 comprises about 70 to about 750 mg omega-6. In an embodiment, one serving of omega-6 comprises about 250 to about 600 mg omega-6. In an embodiment, one serving of omega-6 comprises about 250 to about 500 mg omega-6. In an embodiment, one serving of omega-6 comprises about 300 to about 400 mg omega-6. In an embodiment, one serving of omega-6 comprises about 300 mg omega-6.

In an embodiment, the composition comprises at least one serving of omega-9. In an embodiment, one serving of omega-9 comprises about 70 to about 750 mg omega-9. In an embodiment, one serving of omega-9 comprises about 250 to about 600 mg omega-9. In an embodiment, one serving of omega-9 comprises about 250 to about 500 mg omega-9. In an embodiment, one serving of omega-9 comprises about 300 to about 400 mg omega-9. In an embodiment, one serving of omega-9 comprises about 300 mg omega-9.

In an embodiment, the composition comprises at least one serving of probiotic. In an embodiment, one serving of probiotic is about 1×10⁶ to about 1×10¹² colony forming units (CFU). In an embodiment, 1 serving of probiotic is about 1×10⁷ to about 1×10¹¹ CFU. In an embodiment, 1 serving of probiotic is about 1×10⁸ to about 1×10¹⁰ CFU.

In an embodiment, the composition comprises two or more or all of: at least one serving of vegetable, at least one serving of fruit, at least one serving of omega-3 and at least one serving of probiotics.

In an embodiment, the entrapped or encapsulated probiotic has higher viability compared to the unentrapped or unencapsulated probiotic after treatment with/in one or more or all of the following conditions: i) a temperature of about 4° C. to about 40° C.; ii) a pH of about 1 to about 7; iii) a pH of about 1 to about 5; iv) simulated gastric fluid; v) gastric fluid; vi) digestive enzymes; vii) simulated intestinal fluid; viii) intestinal fluid; ix) transit through the upper gastrointestinal tract; x) the lower gastrointestinal tract; and xi) freeze drying; and xii) sterilization.

In an embodiment, an entrapped probiotic has a higher viability at a temperature of about 4° C. to about 40° C. In an embodiment, an entrapped probiotic has a higher viability at a temperature of about 6° C. to about 35° C. In an embodiment, an entrapped probiotic has a higher viability at a temperature of about 10° C. to about 30° C. In an embodiment, an entrapped probiotic has a higher viability at a temperature of about 15° C. to about 25° C. In an embodiment, an entrapped probiotic has a higher viability at a temperature of about 20° C. to about 25° C.

In an embodiment, an entrapped probiotic has a higher viability after about 1 month to about 24 months storage at a low temperature or room temperature (e.g. 4° C. to about 24° C.). In an embodiment, an entrapped probiotic has a higher viability after about 1 month to about 18 months storage at a low temperature or ˜room temperature. In an embodiment, an entrapped probiotic has a higher viability after about 1 month to about 14 months storage at a low temperature or ˜room temperature. In an embodiment, an entrapped probiotic has a higher viability after about 1 month to about 12 months when stored at storage at a low temperature or ˜room temperature. In an embodiment, an entrapped probiotic has a higher viability after about 1 week to about 2 weeks storage at high temperature (e.g. about 30° C. to about 40° C.).

In an embodiment, an entrapped probiotic has a higher viability at a pH of about 1 to about 7. In an embodiment, an entrapped probiotic has a higher viability at a pH of about 1 to about 6. In an embodiment, an entrapped probiotic has a higher viability at a pH of about 1 to about 5. In an embodiment, an entrapped probiotic has a higher viability at a pH of about 1 to about 4. In an embodiment, an entrapped probiotic has a higher viability at a pH of about 1 to about 3. In an embodiment, an entrapped probiotic has a higher viability at a pH of about 1 to about 2.

In an embodiment, an entrapped probiotic has a higher viability in the presence of simulated gastric fluid. In an embodiment, an entrapped probiotic has a higher viability in the presence of gastric fluid. In an embodiment, an entrapped probiotic has a higher viability in the presence of digestive enzymes. In an embodiment, an entrapped probiotic has a higher viability in the presence of simulated intestinal fluid. In an embodiment, an entrapped probiotic has a higher viability in the presence of the lower gastrointestinal tract.

In an embodiment, an encapsulated probiotic has a higher viability at a temperature of about 4° C. to about 44° C. In an embodiment, an encapsulated probiotic has a higher viability at a temperature of about 4° C. to about 40° C. In an embodiment, an encapsulated probiotic has a higher viability at a temperature of about 6° C. to about 35° C. In an embodiment, an encapsulated probiotic has a higher viability at a temperature of about 10° C. to about 30° C. In an embodiment, an encapsulated probiotic has a higher viability at a temperature of about 15° C. to about 25° C. In an embodiment, an encapsulated probiotic has a higher viability at a temperature of about 20° C. to about 25° C.

In an embodiment, an encapsulated probiotic has a higher viability at a pH of about 1 to about 7. In an embodiment, an encapsulated probiotic has a higher viability at a pH of about 1 to about 6. In an embodiment, an encapsulated probiotic has a higher viability at a pH of about 1 to about 5. In an embodiment, an encapsulated probiotic has a higher viability at a pH of about 1 to about 4. In an embodiment, an encapsulated probiotic has a higher viability at a pH of about 1 to about 3. In an embodiment, an encapsulated probiotic has a higher viability at a pH of about 1 to about 2.

In an embodiment, an encapsulated probiotic has a higher viability after treatment with/in simulated gastric fluid. In an embodiment, an encapsulated probiotic has a higher viability after treatment with/in gastric fluid. In an embodiment, an encapsulated probiotic has a higher viability after treatment with/in digestive enzymes. In an embodiment, an encapsulated probiotic has a higher viability after treatment with/in simulated intestinal fluid. In an embodiment, an encapsulated probiotic has a higher viability when in the lower gastrointestinal tract. In an embodiment, an encapsulated probiotic has a higher viability after treatment/during freeze drying.

In an embodiment, the probiotic is one or more of the following: i) a beneficial bacteria; ii) a lactic acid bacteria; iii) a bacteria which produces one or more short chain fatty acid (SCFA) when in the gastrointestinal tract; iv) a bacteria that assists with the production of one of more SCFA when in the gastrointestinal tract; v) isolated from a Brassicaceae; vi) isolated from broccoli; vii) isolated from Daucus carota; and viii) an autochonthous bacteria from the biomass.

As used herein, an “autochonthous bacteria” refers to a bacteria from the biomass as described herein. The bacteria may be present on the biomass during preparation of the composition as described herein or isolated from the biomass, propagated (which may include storage and propagation) and added during preparation of the composition as described herein.

In an embodiment, the lactic acid bacteria is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is Lactobacillus.

In an embodiment, the lactic acid bacteria is selected from one or more of: Leuconostoc mesenteroides, Lactobacillus plantarum, Lactobacillus pentosus, Lactobacillus brevis, Lactococus lactis, Pediococcus pentosaceus, Lactobacillus rhamnosus and Pedicoccus acidilacti.

In an embodiment, the lactic acid bacteria is selected from one or more of: i) BF1 deposited under V17/021729 on 25 Sep. 2017 at the National Measurement Institute 1/153 Bertie Street, Port Melbourne, Victoria, Australia 3207; ii) BF2 deposited under V17/021730 on 25 Sep. 2017 at the National Measurement Institute 1/153 Bertie Street, Port Melbourne, Victoria, Australia 3207; iii) B1 deposited under V17/021731 on 25 Sep. 2017 at the National Measurement Institute 1/153 Bertie Street, Port Melbourne, Victoria, Australia 3207; iv) B2 deposited under V17/021732 on 25 Sep. 2017 at the National Measurement Institute 1/153 Bertie Street, Port Melbourne, Victoria, Australia 3207; v) B3 deposited under V17/021733 on 25 Sep. 2017 at the National Measurement Institute 1/153 Bertie Street, Port Melbourne, Victoria, Australia 3207; vi) B4 deposited under V17/021734 on 25 Sep. 2017 at the National Measurement Institute 1/153 Bertie Street, Port Melbourne, Victoria, Australia 3207; and vii) B5 deposited under V17/021735 on 25 Sep. 2017 at the National Measurement Institute 1/153 Bertie Street, Port Melbourne, Victoria, Australia 3207.

In an embodiment, the bacteria that produces one or more SCFA is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus, Clostridium cluster XIVa, Bifidobacterium spp., Bacteroidetes and Negativicutes classes of Firmicutes

In an embodiment, the composition further comprises oil. In an embodiment, the oil is entrapped or encapsulated in the matrix. In an embodiment, the entrapped or encapsulated oil is resistant to degradation compared to the unentrapped or unencapsulated oil.

In an embodiment, the oil is resistant to degradation by one or more of: oxygen, temperature, pH, moisture, light, and pro-oxidants.

In an embodiment, the omega-3 fatty acids/unsaturated fatty acids in the entrapped or encapsulated oil is resistant to degradation for at least one week when stored at about 40° C. compared to unentrapped or encapsulated oil. In an embodiment, the omega-3 fatty acids/unsaturated fatty acids in the entrapped or encapsulated oil is resistant to degradation for at least two weeks when stored at about 40° C. compared to unentrapped or encapsulated oil. In an embodiment, at least 80% of the omega-3 fatty acids/unsaturated fatty acids in the entrapped or encapsulated oil is present after at least two weeks storage at about 40° C. In an embodiment, at least 75% of the omega-3 fatty acids/unsaturated fatty acids in the entrapped or encapsulated oil is present after at least two weeks storage at about 40° C. In an embodiment, at least 70% of the omega-3 fatty acids/unsaturated fatty acids in the entrapped or encapsulated oil is not oxidised after at least two weeks storage at about 40° C. In an embodiment, at least 60% of the omega-3 fatty acids/unsaturated fatty acids in the entrapped or encapsulated oil is present after at least two weeks storage at about 40° C. In an embodiment, at least 50% of the omega-3 fatty acids/unsaturated fatty acids in the entrapped or encapsulated oil is present after at least two weeks storage at about 40° C.

In an embodiment, the oil encapsulated in the composition as described herein is about 500% to about 4000% more resistant to oxygen degradation than unentrapped or unencapsulated oil when time to IP is compared. In an embodiment, the oil encapsulated in the composition as described herein is about 500% to about 3000% more resistant to oxygen degradation than unentrapped or unencapsulated oil when time to IP is compared. In an embodiment, the oil encapsulated in the composition as described herein is about 500% to about 2000% more resistant to oxygen degradation than unentrapped or unencapsulated oil when time to IP is compared. In an embodiment, the oil encapsulated in the composition as described herein is about 800% to about 2000% more resistant to oxygen degradation than unentrapped or unencapsulated oil when time to IP is compared. In an embodiment, the oil encapsulated in the composition as described herein is about 800% to about 1500% more resistant to oxygen degradation than unentrapped or unencapsulated oil when time to IP is compared. In an embodiment, the oil encapsulated in the composition as described herein is about 900% to about 1300% more resistant to oxygen degradation than unentrapped or unencapsulated oil when time to IP is compared

In an embodiment, the oil encapsulated in the composition is more resistant to oxygen degradation for at least 3 months compared to unencapsulated oil when stored at about 24° C. In an embodiment, the oil encapsulated in the composition is more resistant to oxygen degradation for at least 6 months compared to unencapsulated oil when stored at about 24° C. In an embodiment, the oil encapsulated in the composition is more resistant to oxygen degradation for at least 12 months compared to unencapsulated oil when stored at about 24° C. In an embodiment, the oil encapsulated in the composition is more resistant to oxygen degradation for at least 18 months compared to unencapsulated oil when stored at about 24° C. In an embodiment, the oil encapsulated in the composition is more resistant to oxygen degradation for at least 24 months compared to unencapsulated oil when stored at about 24° C.

In an embodiment, the oil encapsulated in the composition is more resistant to oxygen degradation for at least 3 months compared to unencapsulated oil. In an embodiment, the oil encapsulated in the composition is more resistant to oxygen degradation for at least 6 months compared to unencapsulated oil. In an embodiment, the oil encapsulated in the composition is more resistant to oxygen degradation for at least 12 months compared to unencapsulated oil. In an embodiment, the oil encapsulated in the composition is more resistant to oxygen degradation for at least 18 months compared to unencapsulated oil. In an embodiment, the oil encapsulated in the composition is more resistant to oxygen degradation for at least 24 months compared to unencapsulated oil.

In an embodiment, the oil encapsulated in the composition is more resistant to temperature degradation for at least 3 months compared to unencapsulated oil. In an embodiment, the oil encapsulated in the composition is more resistant to temperature degradation for at least 6 months compared to unencapsulated oil. In an embodiment, the oil encapsulated in the composition is more resistant to temperature degradation for at least 12 months compared to unencapsulated oil. In an embodiment, the oil encapsulated in the composition is more resistant to temperature degradation for at least 18 months compared to unencapsulated oil. In an embodiment, the oil encapsulated in the composition is more resistant to temperature degradation for at least 24 months compared to unencapsulated oil.

In an embodiment, the oil encapsulated in the composition is more resistant to moisture degradation for at least 3 months compared to unencapsulated oil. In an embodiment, the oil encapsulated in the composition is more resistant to moisture degradation for at least 6 months compared to unencapsulated oil. In an embodiment, the oil encapsulated in the composition is more resistant to moisture degradation for at least 12 months compared to unencapsulated oil. In an embodiment, the oil encapsulated in the composition is more resistant to moisture degradation for at least 18 months compared to unencapsulated oil. In an embodiment, the oil encapsulated in the composition is more resistant to moisture degradation for at least 24 months compared to unencapsulated oil.

In an embodiment, the oil encapsulated in the composition is more resistant to pH degradation for at least 3 months compared to unencapsulated oil. In an embodiment, the oil encapsulated in the composition is more resistant to pH degradation for at least 6 months compared to unencapsulated oil. In an embodiment, the oil encapsulated in the composition is more resistant to pH degradation during gastrointestinal transit than unencapsulated oil.

In an embodiment, the oil comprises one or more fatty acid/s as described herein. In an embodiment, the fatty acid is selected from one or more of: omega-3, omega-6 or an omega-9 fatty acid. In an embodiment, the omega-3 fatty acid is one or more of α-linolenic acid (ALA), eicosapentaenoic acid (EPA), docosapentaenoic acid (DPA) and docosahexaenoic acid (DHA). In an embodiment, the oil is selected from one or more of: fish oil, krill oil, algal oil, marine oil, fungal oil, nut or seed oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, medium chain triglycerides and grapeseed oil. In an embodiment, the fish oil or marine oil is selected from one or more of: tuna oil, herring oil, mackerel oil, sardine oil, cod liver oil, menhaden oil, shark oil, algal oil, squid oil, and squid liver oil.

In an embodiment, the composition further comprises biomass comprising protein and carbohydrate from at least one further single species of organism.

In an embodiment, one or more components of the biomass or further biomass is a prebiotic. In an embodiment, the prebiotic is a fibre.

In an embodiment, the biomass or further biomass is from the Plantae or Fungi Kingdom. In an embodiment, the Plantae is selected from: Brassicaceae, Cannabis Asparagaceae, Arecaceae, Myrtaceae, Rosaceae, Musaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae, Caesalpinioideae, Compositae, Amaranthaceae, Chenopodiacae, Malvaceae, Amarylidaceae, Fabaceae, Arecaceae and Poaceae. In an embodiment, the Plantae is Brassicaceae. In an embodiment, the Brassicaceae is broccoli.

In an embodiment, the composition increases the gastrointestinal level of one or more SCFA in a subject. In an embodiment, the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoi acid), isobutyric acid (2-methylpropanoic acid), valeric acid (pentanoic acid), isovaleric acid (3-methylbutanoic acid), and caproic acid (hexanoic acid). In an embodiment, the SCFA is selected from one or more or all of: butyrate, propionate and acetate. In an embodiment, the SCFA is butyrate. In an embodiment, the SCFA is propionate. In an embodiment, the SCFA is acetate.

In an embodiment, administering a composition or product as described herein to a subject increases the SCFA in stool samples by about 10% to about 100%. In an embodiment, administering a composition or product as described herein to a subject increases the SCFA in stool samples by about 20% to about 100%. In an embodiment, administering a composition or product as described herein to a subject increases the SCFA in stool samples by about 30% to about 100%. In an embodiment, administering a composition or product as described herein to a subject increases the SCFA in stool samples by about 40% to about 100%. In an embodiment, administering a composition or product as described herein to a subject increases the SCFA in stool samples by about 50% to about 100%. In an embodiment, administering a composition or product as described herein to a subject increases the SCFA in stool samples by about 60% to about 100%. In an embodiment, administering a composition or product as described herein to a subject increases the SCFA in stool samples by about 70% to about 100%. In an embodiment, administering a composition or product as described herein to a subject increases the SCFA in stool samples by about 80% to about 100%. In an embodiment, administering a composition or product as described herein to a subject increases the SCFA in stool samples by about 90% to about 100%.

In an embodiment, the prebiotic increases the SCFA level about 5 to about 48 hours after administration. In an embodiment, the prebiotic increases the SCFA level about 10 to about 24 hours after administration.

In an embodiment, the composition increases the gastrointestinal level of one or more of: i) lactic acid bacteria, ii) bacteria that produces one or more SCFA; and iii) a bacteria that assists with the production of one of more SCFA. In an embodiment, the bacteria that produces one or more SCFA is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus, Clostridium cluster XlVa. Bifidobacterium spp., Bacteroidetes and Negativicutes classes of Firmicutes

In an embodiment, the composition increases the gastrointestinal level of one or more or all of: Colinsella, Bacillus, Lactobacillus, Lachnospira, Faecalibacterium, Dialister and Veillonella in a subject. In an embodiment, the composition increases the gastrointestinal level of Colinsella in a subject. In an embodiment, the composition increases the gastrointestinal level of Bacillus in a subject. In an embodiment, the composition increases the gastrointestinal level of Lactobacillus in a subject. In an embodiment, the composition increases the gastrointestinal level of Lachnospira in a subject. In an embodiment, the composition increases the gastrointestinal level of Faecalibacterium in a subject. In an embodiment, the composition increases the gastrointestinal level of Dialister in a subject. In an embodiment, the composition increases the gastrointestinal level of Veillonella in a subject. In an embodiment, the gastrointestinal level, is the level in the lower gastrointestinal tract.

In an embodiment, the composition increases the faecal level of one or more or all of: Colinsella, Bacillus, Lactobacillus, Lachnospira, Faecalibacterium, Dialister and Veillonella in a subject. In an embodiment, the composition increases the gastrointestinal level of Colinsella in a subject.

In an embodiment, the composition decreases the gastrointestinal level of one or more or all of: Bacteroides, Parabacteroides, Paraprevotella, Turicibacter, Christensenellaceae, some members of Family Clostridiales, Clostridium, some members of Family Lachnospiraceae, Dorea, Roseburia, some members of the Family Ruminococcaceae, Oscillospira, Ruminococcus, some members of the Family Erysipelotrichaceae, Klebsiella and Trabulsiella in a subject. In an embodiment, the gastrointestinal level, is the level in the lower gastrointestinal tract.

In an embodiment, the composition decreases the gastrointestinal level of Bacteroides. In an embodiment, the composition decreases the gastrointestinal level of Parabacteroides. In an embodiment, the composition decreases the gastrointestinal level of Paraprevotella. In an embodiment, the composition decreases the gastrointestinal level of Turicibacter. In an embodiment, the composition decreases the gastrointestinal level of Christensenellaceae. In an embodiment, the composition decreases the gastrointestinal level of some members of Family Clostridiales. In an embodiment, the composition decreases the gastrointestinal level of Clostridium. In an embodiment, the composition decreases the gastrointestinal level of some members of Family Lachnospiraceae. In an embodiment, the composition decreases the gastrointestinal level of Dorea. In an embodiment, the composition decreases the gastrointestinal level of Roseburia. In an embodiment, the composition decreases the gastrointestinal level of some members of the Family Ruminococcaceae. In an embodiment, the composition decreases the gastrointestinal level of some members of the Family Erysipelotrichaceae. In an embodiment, the composition decreases the gastrointestinal level of Klebsiella. In an embodiment, the composition decreases the gastrointestinal level of Trabulsiella.

In an embodiment, the composition decreases the faecal level of one or more or all of: Bacteroides, Parabacteroides, Paraprevotella, Turicibacter, Christensenellaceae, some members of Family Clostridiales, Clostridium, some members of Family Lachnospiraceae, Dorea, Roseburia, some members of the Family Ruminococcaceae, Oscillospira, Ruminococcus, some members of the Family Erysipelotrichaceae, Klebsiella and Trabulsiella in a subject.

In an embodiment, the composition comprises about 50% to about 90%, or about 60% to about 80%, or about 70% to about 80% biomass. In an embodiment, the composition comprises about 50% to about 90% biomass. In an embodiment, the composition comprises about 60% to about 80% biomass. In an embodiment, the composition comprises about 70% to about 80% biomass.

In an embodiment, the composition comprises about 1×10⁶ CFU/g to about to 1×10¹⁴ CFU/g, about 1×10⁶ CFU/g to about to 1×10¹² CFU/g, or about 1×10⁷ CFU/g to about to 1×10¹¹ CFU/g, 1×10⁸ CFU/g to about to 1×10¹⁰ CFU/g, and 1×10⁹ CFU/g to about to 1×10¹⁰ CFU/g probiotic. In an embodiment, the composition comprises about 1×10⁶ CFU/g to about to 1×10¹² CFU/g probiotic. In an embodiment, the composition comprises about 1×10⁷ CFU/g to about to 1×10¹¹ CFU/g probiotic. In an embodiment, the composition comprises about 1×10⁹ CFU/g to about to 1×10¹⁰ CFU/g probiotic.

In an embodiment, the composition comprises about 5% to about 50%, or about 10% to about 45%, or about 10% to about 40%, or about 15% to about 30%, or about 20% to about 30% w/w oil. In an embodiment, the composition comprises about 5% to about 50%, w/w oil. In an embodiment, the composition comprises about 10% to about 45% w/w oil. In an embodiment, the composition comprises about 10% to about 40% w/w oil. In an embodiment, the composition comprises about 15% to about 30% w/w oil. In an embodiment, the composition comprises about 20% to about 30% w/w oil.

In an embodiment, the composition has an induction period of about 10 to about 300 hours, when measured at 80° C. and a 5 bar initial oxygen pressure. In an embodiment, the composition has an induction period measured using the Oxipres at 80° C. and initial 5 bar oxygen pressure, of about 50 to about 300 hours at 80° C. In an embodiment, the composition has an induction period measured using the Oxipres at 80° C. and initial 5 bar oxygen pressure, of about 80 to about 300 hours at 80° C. In an embodiment, the composition has an induction period measured using the Oxipres at 80° C. and initial 5 bar oxygen pressure, of about 100 to about 300 hours at 80° C. In an embodiment, the composition has an induction period measured using the Oxipres at 80° C. and initial 5 bar oxygen pressure, of at least 10 hours at 80° C. In an embodiment, the composition has an induction period measured using the Oxipres at 80° C. and initial 5 bar oxygen pressure, of at least 50 hours at 80° C. In an embodiment, the composition has an induction period measured using the Oxipres at 80° C. and initial 5 bar oxygen pressure, of at least 100 hours at 80° C.

In an embodiment, moisture content of the composition is about 1 to about 14%. In an embodiment, moisture content of the composition is about 1 to about 10%. In an embodiment, the moisture content of the composition is about 10% or less. In an embodiment, the moisture content of the composition is about 8% or less. In an embodiment, the moisture content of the composition is about 7% or less. In an embodiment, the moisture content of the composition is about 6% or less. In an embodiment, the moisture content of the composition is about 5% or less. In an embodiment, the moisture content of the composition is about 4% or less. In an embodiment, the moisture content of the composition is about 3% or less. In an embodiment, the composition comprises oil. In an embodiment, the composition comprises omega-3 polyunsaturated fatty acid. In an embodiment, the composition comprises an isothiocyanate bioactive.

In an aspect, the product comprises the composition as described herein, or is produced by the method as described herein.

In an embodiment, the product comprises a prebiotic and a probiotic. In an embodiment, the product is a synbiotic. In an embodiment, the product comprises an isothiocyanate and/or glucosinolate.

In an embodiment, the product is vegan. In an embodiment, the vegan product comprises algal oil.

In an embodiment, the product is a cream, gel tablet, liquid, pill, powder or extruded product. In an embodiment, the product is a powder.

In an embodiment, the composition as described herein can be used as is or is a material added to or combined with other materials to from a product (e.g. a food, feed, supplement, cosmetic product, skin care product or a preventative health product).

In an embodiment, the composition as described herein can be used to form a powder (is combined with one or more other powdered ingredients), tablet, liquid, pill, or extruded product. In an embodiment, the powder is extruded. In an embodiment, the powder is compressed e.g. to form a tablet.

In an embodiment, the product is a food. In an embodiment, the product is a food ingredient e.g. infant formulae, children formula, adult formula, yoghurts, beverages, elderly supplement, ultra-high temperature processed (UHT) drinks (e.g. milk), soup, dips, pasta products, bread, snacks and other bakery products processed cheese, and/or animal feed (including aquaculture). In an embodiment, the product is a supplement. In an embodiment, the product is a nutraceutical.

In an embodiment, the composition is a food grade composition. In an embodiment, the composition is included in a food or other product formulation and subject to a further processing treatment to achieve microbial safety,

In an embodiment, the product is suitable for use as a cosmetic or cosmetic ingredient, for example, as a lipstick, cream, lotion, or ointment.

In an embodiment, the product is a powdered supplement. In an embodiment, the powdered supplement is dissolved in water or added to a food, beverage or a meal.

In an embodiment, the product is an animal feed. In an embodiment, the product is an animal feed ingredient. In an embodiment, the product may be added to an animal feed e.g. Novacq prawn feed (CSIRO). The animal can be an aquatic animal such as fish, prawns or livestock. In an embodiment, the product is an animal supplement. In an embodiment, the animal can be a livestock or companion animal.

In an embodiment, the product is an aquaculture feed. In an embodiment, the product is an aquaculture feed ingredient. In an embodiment, the product is an aquaculture.

### Pharmaceutical Compositions

In an aspect, the present invention provides a pharmaceutical composition comprising a composition as described herein, or product as described herein for use in promoting health in a subject.

In an aspect, the present invention provides a pharmaceutical composition comprising a composition as described herein, or product as described herein for use in promoting health of the gut microbiome in a subject.

In an aspect, the present invention provides a pharmaceutical composition comprising a composition as described herein, or product as described herein for treating and/or preventing microbial dysbiosis in the gastrointestinal tract of a subject.

In an aspect, the present invention provides a pharmaceutical composition comprising a composition as described herein, or product as described herein for treating and/or preventing inflammation in a subject.

In an aspect, the present invention provides a pharmaceutical composition comprising a composition as described herein, or product as described herein for treating and/or preventing diabetes in a subject.

In an aspect, the present invention provides a pharmaceutical composition comprising a composition as described herein, or product as described herein for treating and/or preventing asthma in a subject.

In an aspect, the present invention provides a pharmaceutical composition comprising a composition as described herein, or product as described herein for increasing nutrient absorption or nutrient utilisation in a subject.

In an embodiment, the pharmaceutical composition comprises one or more pharmaceutically acceptable excipients. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In an embodiment, the pharmaceutical compositions as described herein may comprise one or more further active ingredients.

### Post-Treating

In an embodiment, the composition or product as described herein is post-treated to sterilize the product. This may occur for example at low heat for probiotics that are sensitive to treatment with high temperatures. This may occur at high heat for probiotics which retain viability at higher temperatures.

### Promoting Health

The present invention provides compositions, products and methods for promoting the health of a subject. As used herein “health” refers to the condition of a subject's body and the extent to which the subject's body is resistant to an illness or free from an illness.

As used herein “promoting health” refers to increasing, enhancing, inducing, and/or stimulating resistance or resilience to an illness or a reduction in one or more symptoms of an illness.

As used herein “resistance” refers to the insensitivity to a disturbance.

As used herein “resilience” refers to the rate of the recovery after a disturbance.

In an embodiment, promoting health comprises treating or preventing a condition in a subject.

In an embodiment, promoting health comprises treating or preventing one or more symptoms of a condition selected from: diabetes, inflammation, metabolic dysfunction, asthma, allergy and cancer.

In an embodiment, promoting health comprises promoting one or more of: gut health, immune system health, cardiovascular health, central nervous system function, cognition, metabolic health, nutrient absorption, nutrient utilisation, reducing acidosis, daily increase in body weight, an increase in total body weight, resistance to pathogen colonisation, skeletal health, liver health, blood sugar control and skin health. In an embodiment, promoting health comprises promoting gut health. In an embodiment, promoting health comprises promoting immune system health. In an embodiment, promoting health comprises promoting cardiovascular health. In an embodiment, promoting health comprises promoting central nervous system function. In an embodiment, promoting health comprises promoting cognition. In an embodiment, promoting health comprises promoting metabolic health. In an embodiment, promoting health comprises promoting nutrient absorption. In an embodiment, promoting health comprises promoting nutrient utilisation. In an embodiment, promoting health comprises reducing acidosis. In an embodiment, promoting health comprises promoting daily increase in body weight. In an embodiment, promoting health comprises promoting an increase in total body weight. In an embodiment, promoting health comprises promoting resistance to pathogen colonisation. In an embodiment, promoting health comprises promoting skeletal health. In an embodiment, promoting health comprises promoting liver health. In an embodiment, promoting health comprises promoting blood sugar control. In an embodiment, promoting health comprises promoting skin health.

In an embodiment, promoting gut health comprises reducing or preventing one or more symptoms of a gut health associated condition selected from one or more of: irritable bowel syndrome, inflammatory bowel disease, Crohn's disease, colorectal cancer, gut leakiness, non-alcoholic fatty liver disease, metabolic syndrome, obesity, small intestinal bacterial overgrowth (SIBO), gastroenteritis, gut microbial dysbiosis, reduced gut microbial diversity, antibiotic treatment, post-surgery recovery, food intolerance, diarrhoea, gastritis, diverticulitis, flatulence, constipation, functional gut disorders and functional gastrointestinal and motility disorders.

In an embodiment, the functional gut disorder is selected from one or more of: functional abdominal bloating/distension, functional constipation, functional diarrhoea, unspecified functional bowel disorder, opioid-induced constipation, centrally mediated abdominal pain syndrome, narcotic bowel syndrome, opioid-induced hyperalgesia, functional pancreatic sphincter of oddi disorder, biliary pain, faecal incontinence, functional anorectal pain, and functional defecation disorders.

In an embodiment, the functional gastrointestinal and motility disorders is selected from one or more of: gastroesophageal reflux disease, intestinal dysmotility, intestinal pseudo-obstruction, small bowel bacterial overgrowth, constipation, outlet obstruction type constipation (pelvic floor dyssynergia), diarrhoea, faecal incontinence, hirschsprung's disease, gastroparesis and achalasia.

In an embodiment, promoting health comprises promoting health of the gut microbiome in a subject. In an embodiment, promoting health of the gut microbiome comprises one or more of: increasing the level and/or activity of one or more beneficial bacteria, decreasing or maintaining the level and/or activity of one or more non-beneficial bacteria, increasing the resistance of the gut microbiome, increasing the resilience of the gut microbiome, and increasing the diversity of the gut microbiome. In an embodiment, promoting health of the gut microbiome comprises increasing the level and/or activity of one or more beneficial bacteria. In an embodiment, promoting health of the gut microbiome comprises decreasing or maintaining the level and/or activity of one or more non-beneficial bacteria. In an embodiment, promoting health of the gut microbiome comprises increasing the resistance of the gut microbiome. In an embodiment, promoting health of the gut microbiome comprises increasing the resilience of the gut microbiome. In an embodiment, promoting health of the gut microbiome comprises increasing the diversity of the gut microbiome.

As used herein “resistance of the gut microbiome” refers to the insensitivity of the gut microbiome to a disturbance. As used herein “resilience of the gut microbiome” refers to the rate of the recovery of the gut microbiome after a disturbance (e.g. a disturbance may reduce the number or type of microorganism in the microbiome). In an embodiment, the beneficial bacteria assists in the generation of SCFA in the gastrointestinal tract. In an embodiment, the beneficial bacteria produces one or more SCFA (e.g. Faecalibacterium). In an embodiment, the beneficial bacteria produces butyrate. In an embodiment, the beneficial bacteria strengthens the integrity of the of the mucus barrier (e.g. Akkermansia muciniphila).

In an embodiment, the beneficial bacteria is selected from one or more or all of: lactic acid bacteria, Bifidobacteria, Bacteroidetes, Baciullus, Streptococcus, Escherichia, and Enterococcus.

In an embodiment, the lactic acid bacteria is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella. In an embodiment, the lactic acid bacteria is selected from one or more or all of: Lactobacillus plantarum, Leuconostoc mesenteroides, Lactobacillus rhamnosus, Lactobacillus pentosus, Lactobacillus brevis, Lactococus lactis, Lactobacillus acidophilus, Lactobacillus brevis, Lactobacillus casei, Lactobacillus delbrueckii, Lactobacillus fermentum, Lactobacillus gasseri, Lactobacillus johnsonii, Lactobacillus lactis, Lactobacillus paracasei, Lactobacillus reuteri, Pediococcus pentosaceus and Pedicoccus acidilacti.

In an embodiment, the lactic acid bacteria is selected from one or more or all of: i) BF1 deposited under V17/021729 on 25 Sep. 2017 at the National Measurement Institute 1/153 Bertie Street, Port Melbourne, Victoria, Australia 3207; ii) BF2 deposited under V17/021730 on 25 Sep. 2017 at the National Measurement Institute 1/153 Bertie Street, Port Melbourne, Victoria, Australia 3207; iii) B1 deposited under V17/021731 on 25 Sep. 2017 at the National Measurement Institute 1/153 Bertie Street, Port Melbourne, Victoria, Australia 3207; iv) B2 deposited under V17/021732 on 25 Sep. 2017 at the National Measurement Institute 1/153 Bertie Street, Port Melbourne, Victoria, Australia 3207; v) B3 deposited under V17/021733 on 25 Sep. 2017 at the National Measurement Institute 1/153 Bertie Street, Port Melbourne, Victoria, Australia 3207; vi) B4 deposited under V17/021734 on 25 Sep. 2017 at the National Measurement Institute 1/153 Bertie Street, Port Melbourne, Victoria, Australia 3207; and vii) B5 deposited under V17/021735 on 25 Sep. 2017 at the National Measurement Institute 1/153 Bertie Street, Port Melbourne, Victoria, Australia 3207.

In embodiment, the Bifidobacteria is selected from one or more of: Bifidobacteria adolescentis, Bifidobacteria animalis, Bifidobacteria bifidum, Bifidobacteria breve, Bifidobacteria infantis, Bifidobacteria longum, and Bifidobacteria thermophilum.

In embodiment, the Baciullus is selected from one or more of: Baciullus cereus, Baciullus clausii, Baciullus coagulans, Baciullus licheniformis, Baciullus pumulis and Baciullus subtilis.

In embodiment, the Streptococcus is Streptococcus thermophiles. In embodiment, the Escherichia is beneficial strain of Escherichia coli.

In an embodiment, the bacteria that produces one or more SCFA is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus, Clostridium cluster XIVa. Bifidobacterium spp., Bacteroidetes and Negativicutes classes of Firmicutes.

In embodiment, the Enterococcus is Enterociccus faecium.

In an embodiment, the non-beneficial bacteria is a pathogenic strain of bacteria.

In an embodiment, the non-beneficial bacteria is a pathogenic strain of bacteria selected from one or more of: Escherichia coli, Enterococcus, Helicobacter pylori, Clostridium, Vibrio cholerae, Bacteroides fragilis, Fusobacterium, Staphylococcus (e.g. pneumoniae), Legionella, Haemophilus, Pseudomonas, Prevotella, Salmonella, Campylobacter, and Shigella, Listeria.

In an embodiment, non-beneficial bacteria is a pathogenic strain of Escherichia coli.

In an embodiment, promoting gut health comprises modulating microbial diversity in the gastrointestinal tract of a subject. In an embodiment, modulating microbial diversity comprises increasing microbial diversity. This may occur, for example after a disturbance which reduces the microbial diversity of the gastrointestinal tract (e.g. antibiotic treatment).

In an embodiment, promoting gut health comprises treating and/or preventing microbial dysbiosis in the gastrointestinal tract of a subject. As used herein “microbial dysbiosis” refers to an imbalance in the microbiome that is associated with a disease, precedes a disease or occurs as the result of a disease. The imbalance, for example, could be a gain or loss of members of the microbiome community or changes in relative abundance of members of the microbiome community.

In the context of animal health, such as livestock or companion animals, promoting health may comprise one or more of the following: gut health, immune system health, cardiovascular health, central nervous system function, cognition, metabolic health, nutrient absorption, nutrient utilisation, feed utilisation, reducing the requirement for antibiotic treatment, reducing acidosis, daily increase in body weight, an increase in total body weight, resistance to pathogen colonisation, skeletal health, liver health, blood sugar control and skin health.

In an embodiment in the context of animal health, promoting health comprises one or more of the following: nutrient absorption, nutrient utilisation, reducing acidosis, daily increase in body weight, an increase in total body weight, and resistance to pathogen colonisation, increasing feed efficacy. In an embodiment, the animal is a livestock. In an embodiment, the livestock is an aquaculture livestock. In an embodiment, the animal is a companion animal. In an embodiment, the compositions, products and methods as described herein increase nutrient absorption. In an embodiment, the compositions, products and methods as described herein increase nutrient utilisation. In an embodiment, the compositions, products and methods as described herein increase feed efficacy. In an embodiment, the compositions, products and methods as described herein can improve the quality (e.g. size, nutrient composition, taste, texture, time required before harvesting) of livestock derived products. In an embodiment, the livestock derived product is selected from one or more of: coat, hide, milk, meat or eggs. In an embodiment, the livestock derived product is selected from one or more of: milk, meat or eggs.

### Administration

A variety of routes of administration are possible for the compositions, products and methods as described herein, including but not limited to enteral, dietary, parenteral, and topically. In an embodiment, the composition or product as described herein is administered enterally. As used herein “enterally” or “enteral” comprises passing through the gastrointestinal tract. In an embodiment, enteral administration comprises oral administration. In an embodiment, enteral administration comprises rectal administration. In an embodiment, rectal administration may be selected from one or more of: suppository, enema, via colonoscope or other medical equipment and faecal transplantation. In an embodiment, the composition or product as described herein is administered parenterally. In an embodiment, the composition or product as described herein is administered topically.

In an embodiment, the present invention provides a faecal microbiota suitable for transplantation into a subject, wherein the faecal microbiota has been isolated from a subject administered a composition comprising a probiotic entrapped or encapsulated in a matrix comprising protein and carbohydrate from a non-fermented biomass from a single species of organism.

In an embodiment, the present invention provides a digesta microbiota suitable for transplantation into a subject, wherein the digesta microbiota was isolated from a subject administered a composition comprising a probiotic entrapped or encapsulated in a matrix comprising protein and carbohydrate from a non-fermented biomass from a single species of organism.

### Glucosinolates

As used herein “glucosinolate” refers to a secondary metabolite found at least in the Brassicaceae family that share a chemical structure consisting of a β-D-glucopyranose residue linked via a sulfur atom to a (Z)-N-hydroximinosulfate ester, plus a variable R group derived from an amino acid as described in Halkier et al. (2006). Examples of glucosinolates are provided in Halkier et al. (2006) and Agerbirk et al. (2012). The hydrolysis of glucosinolate can produce isothiocyanates, nitriles, epithionitrile, thiocyanate and oxazolidine-2-thione. Many glucosinolates play a role in plant defence mechanisms against pests and disease.

Glucosinolates are stored in Brassicaceae in storage sites. As used herein, a “storage site” is a site within the Brassicaceae where glucosinolates are present and myrosinase is not present.

As used herein “myrosinase” also referred to as “thioglucosidase”, “sinigrase”, or “sinigrinase” refers to a family of enzymes (EC 3.2.1.147) involved in plant defence mechanisms that can cleave thio-linked glucose. Myrosinases catalyze the hydrolysis of glucosinolates resulting in the production of isothiocyanates. Myrosinase is stored sometimes as myrosin grains in the vacuoles of particular idioblasts called myrosin cells, but have also been reported in protein bodies or vacuoles, and as cytosolic enzymes that tend to bind to membranes. Thus, in an embodiment, myrosinase is stored in a myrosin cell in Brassicaceae.

In an embodiment, pre-treating as described herein improves the access of myrosinase to a glucosinolate. As used herein “improves the access” or “access is improved” refers to increasing the availability of glucosinolate to the myrosinase enzyme allowing for the production of an isothiocyanate. In an embodiment, access is improved by the release of a glucosinolate from a glucosinolate storage site. In an embodiment, the glucosinolate storage site is mechanically ruptured (i.e. by maceration) or enzymatically degraded. In an embodiment, glucosinolate is released from a glucosinolate storage site by the activity of one or more polysaccharide degrading enzymes e.g. a cellulase, hemicellulase, pectinase and/or glycosidase. In an embodiment, access is improved by allowing the entry of myrosinase into a glucosinolate storage site. In an embodiment, access is improved by the release of myrosinase from myrosin cells. In an embodiment, about 10% to about 90% of a glucosinolate is released from a glucosinolate storage site. In an embodiment, about 10% to about 80% of a glucosinolate is released from a glucosinolate storage site. In an embodiment, about 30% to about 70% of a glucosinolate is released from a glucosinolate storage site. In an embodiment, about 40% to about 60% of a glucosinolate is released from a glucosinolate storage site. In an embodiment, about 45% to about 55% of a glucosinolate is released from a glucosinolate storage site.

In an embodiment, the Brassicaceae comprises one or more glucosinolate/s selected from an aliphatic, indole or aromatic glucosinolate.

In an embodiment, the aliphatic glucosinolate is selected from one or more of glucoraphanin (4-Methylsulphinylbutyl or glucorafanin), sinigrin (2-Propenyl), gluconapin (3-Butenyl), glucobrassicanapin (4-Pentenyl), progoitrin (2(R)-2-Hydroxy-3-butenyl, epiprogoitrin (2(S)-2-Hydroxy-3-butenyl), gluconapoleiferin (2-Hydroxy-4-pentenyl), glucoibervirin (3-Methylthiopropyl, glucoerucin (4-Methylthiobutyl), dehydroerucin (4-Methylthio-3-butenyl, glucoiberin (3-Methylsulphinylpropyl), glucoraphenin (4-Methylsulphinyl-3-butenyl), glucoalyssin (5-Methylsulphinylpentenyl), and glucoerysolin (3-Methylsulphonylbutyl, 4-Mercaptobutyl).

In an embodiment, the indole glucosinolate is selected from one or more of glucobrassicin (3-Indolyl methyl), 4-hydroxyglucobrassicin (4-Hydroxy-3-indolylmethyl), 4-methoxyglucobrassicin (4-Methoxy-3-indolylmethyl), and neoglucobrassicin (1-Methoxy-3-indolylmethyl).

In an embodiment, the indole glucosinolate is selected from one or more of Glucotropaeolin (Benzyl) and Gluconasturtiin (2-Phenylethyl).

In an embodiment, the Brassicaceae comprises one or more glucosinolate/s selected from benzylglucosinolate, allylglucosinolate and 4-methylsulfinylbutyl. In an embodiment, the glucosinolate is glucoraphanin (4-Methylsulphinylbutyl). In an embodiment, the glucosinolate is glucobrassicin (3-Indolylmethyl).

In an embodiment, pre-treating as described herein increases the extractable glucosinolate content compared to the extractable glucosinolate content of the biomass before pre-treatment.

As used herein “extractable glucosinolate content” refers to the level of glucosinolate accessible in the Brassicaceae for conversion to isothiocyanate. Excluding conversion into nitriles and other compounds the expected maximum yield of isothiocyanate from 1 mole of glucosinolate is 1 mole of isothiocyanate (1 mole of glucosinolate can maximally be converted to 1 mole of isothiocyanate, 1 mole of glucose and 1 mole of sulphate ion). Thus, in one example, the extractable glucoraphanin content of a commercial broccoli cultivar is 3400 μmol glucoraphanin/kg dw and the expected maximum yield of sulforaphane from the commercial broccoli cultivar is 3400 μmol sulforaphane/kg dw.

### Isothiocyanates

As used herein “isothiocyanate” refers to sulphur containing phytochemicals with the general structure R—N═C═S which are a product of myrosinase activity upon a glucosinolate and bioactive derivatives thereof. In an embodiment, the isothiocyanate is sulforaphane (1-isothiocyanato-4-methylsulfinylbutane). In an embodiment, the isothiocyanate is allyl isothiocyanate (3-isothiocyanato-1-propene). In an embodiment, the isothiocyanate is benzyl isothiocyanate. In an embodiment, the isothiocyanate is phenethyl isothiocyanate. In an embodiment, the isothiocyanate is 3-Butenyl isothiocyanate. In an embodiment, the isothiocyanate is 5-vinyl-1,3-oxazolidine-2-thione. In an embodiment, the isothiocyanate is 3-(methylthio)propyl isothiocyanate. In an embodiment, the isothiocyanate is 3-(methylsulfinyl)-propyl isothiocyanate. In an embodiment, the isothiocyanate is 4-(methylthio)-butyl isothiocyanate. In an embodiment, the isothiocyanate is 1-methoxyindol-3-carbinol isothiocyanate. In an embodiment, the isothiocyanate is 2-phenylethyl isothiocyanate. In an embodiment, the isothiocyanate is iberin,

### EXAMPLES

### Example 1

### Materials and Methods

### Chemicals and Reagents

Broccoli puree (BP)
Algal oil (AO) (OIL ALGAL Refined DHA) was purchased from Cargill, Corn Milling North America.
Skim milk powder (SMP) from NZMP.
Epigallocatechin gallate (EGCG) powder (>95%), was provided by Sanfull Biological Technology Co., Ltd (Hunan, China).
Tuna oil (TO) (HiDHA® 25N Food (Tuna oil)) was purchased from Nu-Mega

Ingredients Pty Ltd.
Broccoli stem and leaves (BSL).
Lactobacillus rhamnosus GG (LGG) used as a model probiotic bacteria was donated by Chr.Hansen (Horsholm, Denmark).

### Formulations

Formulations comprising one or more of LGG, BP, AO, and SMP were prepared for the studies, the composition and amounts based on dried weight is provided in Table 2.

### Preparation of Broccoli Puree or Skim Milk Protein Comprising Formulations

Broccoli florets were homogenized with water (3 parts broccoli to 2 parts of water) for 1 min using a kitchen scale magic bullet blender (Nutribullet pro 900 series, LLC, USA) to produce BP. BP was mixed with water (2 parts BP to 1 part of water) and colloid milled 10 times to reduce the particle size for encapsulation.

BP was diluted to 5% total solids and homogenised with AO for 5 minutes at maximum speed using a Silverson emulsified-mixer to produce an emulsion of BP and algal oil (BP-AO). Alternatively, the diluted BP was homogenised with LGG for 30 seconds at maximum speed using Silverson emulsified-mixer to produce a mixture of BP and LGG (BP-LGG).

TABLE 2
Formulations used for probiotic cell viability
study of Lactobacillus rhamnosus GG.

| | | | | Matrix | | Algal oil | Probiotic |
|---|---|---|---|---|---|---|---|
| | BP | | LGG | amount (g) | | amount (g) | amount (g) |
| | (g, dry | AO | (g, dry | in a dose | | in a dose | in a dose |
| Formulation* | matter) | (g) | matter) | (0.15 g) | | (0.15 g) | (0.15 g) |
| LGG | — | — | 100 | — | | — | 0.15 |
| BP | 100 | — | — | 0.15 | BP | — | — |
| AO | — | 100 | — | — | | 0.15 | — |
| SMP | 100 | — | — | 0.15 | SMP | — | — |
| BP-AO | 90 | 10 | | 0.135 | BP | 0.015 | — |
| BP-LGG | 90 | — | 10 | 0.135 | BP | — | 0.015 |
| BP-LGG-AO | 80 | 10 | 10 | 0.12 | BP | 0.015 | 0.015 |
| SMP-AO | 90 | 10 | — | 0.135 | SMP | 0.015 | — |
| SMP-LGG | 90 | — | 10 | 0.135 | SMP | — | 0.015 |
| SMP-LGG-AO | 80 | 10 | 10 | 0.12 | SMP | 0.015 | 0.015 |

*The formulations comprising LGG were formulated to contain 7.7E+10 colony forming units (CFU) per gram of powder before freeze-drying.

The BP-AO emulsion was further homogenised with LGG for 30 seconds at maximum speed using Silverson emulsified-mixer to produce an emulsion of BP, LGG and AO (BP-LGG-AO).

Skim milk powder was prepared as 25% total solids and hydrated at 50° C. for 1 hr. SMP was homogenised with AO for 5 min at maximum speed using Silverson emulsified-mixer to produce an emulsion of SMP and AO (SMP-AO). Alternatively, SMP was homogenised with LGG for 30 seconds at maximum speed using Silverson emulsified-mixer to produce a mixture of SMP and LGG (SMP-LGG).

The SMP-AO emulsion was further homogenised with LGG for 30s at maximum speed using Silverson emulsified-mixer to produce an emulsion of SMP, LGG and AO (SMP-LGG-AO).

All emulsions and mixtures prepared were freeze-dried into powder and stored at −20° C.

### Storage

All freeze-dried BP comprising formulations and dried LGG cells were stored in open plastic containers inside a desiccator at 25° C. with saturated salt solutions of magnesium chloride (MgCl₂) and magnesium nitrate (Mg(NO₃)₂) to provide atmospheres with relative humidity (RH) of 32% (0.32 aw) and 52% (0.52 aw) respectively.

### Example 2

### Effect of Broccoli and Broccoli-Algal Oil Matrices on the Production of Short Chain Fatty Acid in an In Vitro Colonic Fermentation Model

Studies were conducted to determine the effects of broccoli and broccoli-algal oil matrices on the production of short chain fatty acid/s (SFCA) in an in vitro colonic fermentation model. The first study (series 1) involved freeze-dried powdered formulations comprising broccoli stems and leaves (BSL), tuna oil (TO), Epigallocatechin gallate (EGCG) and a combination thereof. The formulations of the freeze-dried powdered used for the study are provided in Table 3.

Tuna oil and EGCG were used as supplied. For the preparation of different freeze-dried powder formulations, the required amount of BSL was dispersed in water as 5% total solids at 50° C. and stirred for 1 hr using a high shear mixer. The dispersion was mixed with EGCG at 50° C. for 30 min to obtain BSL-EGCG mixture. The BSL-EGCG mixture was followed by an addition of tuna oil, and then was homogenised by Silverson emulsifier-mixer (Silverson L4R, Silverson Machines Ltd., Chesham, Buckinghamshire, UK) for 5 min at maximum speed. The BSL dispersion, BSL-EGCG, BSL-TO, BSL-EGCG-TO were freeze dried and stored at −20° C.

TABLE 3
Formulation of broccoli stem and leaves (BSL), tuna oil (TO),
Epigallocatechin gallate (EGCG) and combinations thereof.

| | | | | BSL | Tuna oil | EGCG |
|---|---|---|---|---|---|---|
| | | | | amount | amount | amount |
| | BSL | | | (g) in a | (g) in a | (g) in a |
| | (g, dry | TO | EGCG | dose | dose | dose |
| Formulation* | matter) | (g) | (g) | (0.15 g) | (0.15 g) | (0.15 g) |
| BSL | 100 | — | — | 0.15 | — | — |
| TO | — | 20 | — | | 0.15 | — |
| EGCG | — | — | 20 | | — | 0.15 |
| BSL-EGCG | 75 | — | 25 | 0.1125 | — | 0.0375 |
| BSL-TO | 75 | 25 | — | 0.1125 | 0.0375 | — |
| BSL-EGCG- | 60 | 20 | 20 | 0.09 | 0.03 | 0.03 |
| TO | | | | | | |

*0.15 g of each formulation was used for in-vitro colonic fermentation.

The second study (series 2) involved broccoli puree (BP) for carrying probiotic LGG, algal oil (AO) and a combination of LGG and AO. Formulations used for the second study are provided in Example 1.

Formulations from series 1 and series 2 were used in an in-vitro fermentation comprising faecal slurries to determine fermentation characteristics of the formulations and the formulations' effect on the composition of gut microbiota. A 0.15 g sample of formulations from series 1 and 2 were individually tested in the in-vitro fermentation model.

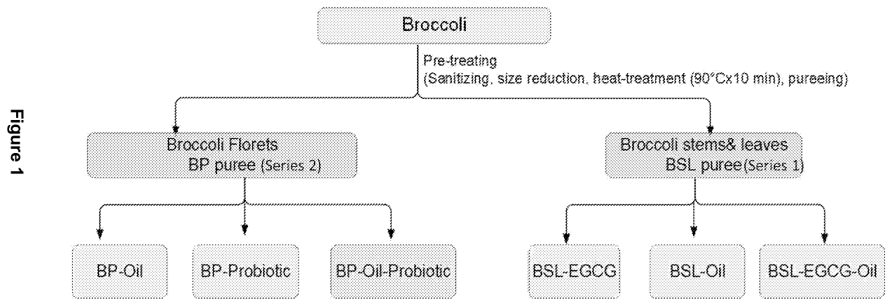

FIG. 1 shows a flowchart for the preparation of broccoli matrices.

### Materials and Methods

Fermentation media: The fermentation media composed of 2 g of peptone, 2 g of yeast extract, 0.1 g of NaCl, 0.04g K₂HPO₃, 0.04 g KH₂PO₄, 0.01 g of MgSO₄.7H₂O, 0.01 g of CaCl₂ 6 H₂O, 2 g of NaHCO₃, 2 mL of Tween, 0.05 g of hemin, 10 μL of vitamin K, 0.5 g of L-cysteine HCl, 0.5 g of bile salt, and 4 mL of resazurin solution (0.025%, w/v) as an anaerobic indicator. The growth medium was autoclaved at 121° C. for 15 min and transfer to anaerobic chamber for overnight equilibration.

pH adjustment of formulation: The pH of the formulations were adjusted by adding 150 mg of the dried formulation to 10 mL fermentation media and adjusting the pH to 6.8 with the addition of H₃PO₄ (1M) or NaOH (1M). The volumes of H₃PO₄ (1M) or NaOH (1M) required for pH adjustment were recorded. For selected samples with added probiotics, the fermentations were also carried out without pH adjustment.

Faecal slurry preparation: Fresh faecal samples were provided by three individual volunteers who were not on dietary restrictions and who had not been antibiotics for at least the last 3 months. Faecal samples were transferred to an anaerobic chamber and large solid particles were filtered. The equivalent amounts of faeces from each donor were combined and diluted to 10% (W/V) with sterile anaerobic phosphate buffered saline (PBS) (0.01M, pH 7.2) to be used as the fermentation starter. The slurry was homogenised and constantly stirred during inoculation into each fermentation test.

In vitro colonic fermentations: The anaerobic batch fermentations were used to assess the effect of samples on fermentation characteristics and composition of gut microbiota. Anaerobic conditions were maintained throughout the set-up of fermentations using an anaerobic chamber (Bactron IV Anaerobic Chamber Sheldon Manufacturing Inc., Cornelius, Oreg., USA) to maximise the bacterial viability of the inoculum. Substrates at a concentration of 1.5% (w/v) in fermentation media were used in each test, and no substrate was added for the blank. Positive and negative control fermentations supplemented with inulin and cellulose at the same concentration were also included. Substrates were inoculated with 10% (w/v) of fresh faecal slurries. All the fermentations were incubated at 37° C. and gently mixed at 80 rpm. After the microbial fermentation, the end products were sampled for SCFA analysis, polyphenol analysis and bacteria population analysis.

SCFA analysis: SCFA were analysed by gas chromatography. Heptanoic acid (30 μL) as internal standard was added to each 0.3 mL of fermentation sample. Samples were mixed thoroughly and centrifuged at 2000 g, 4° C. for 10 min. Then 30 μL of 1 M phosphoric acid was added to 300 μL of the supernatant. Fermentation samples were kept on ice to prevent SCFA volatilisation throughout the processing. Each sample was filtered prior to loading (0.2 μL) into the gas chromatograph (model 7890A; Agilent Technologies, Santa Clara, Calif., USA) equipped with a flame ionisation detector and a capillary column (Zebron ZB-FFAP, 30 m×0.53 mm×1.0 μm, Phenomenex, Lane Cove, NSW, Australia). Helium was used as the carrier gas. The initial oven temperature was 90° C. held for 1 min and was increased at 20° C./min to 190° C. held for 2.5 min; the injector and detector temperature was 210° C.; gas flow and septum purge were at 7.7 and 3.0 mL/min, respectively. A standard SCFA mixture containing acetic, propionic, butyric, iso-butyric, valeric, and iso-valeric was used for calculation, and fatty acid concentrations were calculated in μmol/mL by comparing their peak areas with standards.

### Results of pH Adjustment

The volume of phosphoric acid or NaOH used for pH adjustment is shown in Table 4. The pH of BP-LGG and BP-AO-LGG samples were lower than 6.8. For these samples, two batch fermentations were carried out; one batch without pH adjustment, and the other batch adjusted the pH to 6.8 with NaOH (1M).

The pH value in batch fermentation at 0 hr and 24 hrs are provided in Table 5. The initial pH of fermentation samples was ˜6.8 and this decreased during 24 hrs fermentation. A major factor tending to reduce colonic pH is the production of SCFA by microbial fermentation of dietary carbohydrate and proteins. A decrease in pH because of the enhanced formation of fermentation acids could also lead to changes in the microbiota population (Walker et al., 2005).

TABLE 4
The volume of phosphoric acid (1M) or NaOH (1M)
(μl) to standardize the starting pH at 6.8.

| | | Volume of phosphoric | Volume of NaOH |
|---|---|---|---|
| Substrates | pH | acid (μL) | (μL) |
| Series 1 | | | |
| Without substrate | 6.8 | | |
| Inulin | 8.36 | 65 | |
| Cellulose | 8.33 | 65 | |
| BSL | 7.53 | 50 | |
| Tuna oil (TO) | 8.38 | 65 | |
| EGCG | 7.16 | 50 | |
| BSL-EGCG | 7.74 | 70 | |
| BSL-TO | 7.7 | 50 | |
| BSL-EGCG-TO | 7.59 | 50 | |
| Series 2 | | | |
| LGG | 7.19 | 55 | |
| BP | 7.59 | 50 | |
| SMP | 8 | 70 | |
| Algal oil (AO) | 8.4 | 65 | |
| BP-AO | 7.63 | 40 | |
| SMP-AO | 7.91 | 65 | |
| BP-LGG | 6.26 | | 50 or without |
| | | | addition |
| SMP-LGG | 7.74 | 70 | |
| BP-AO-LGG | 6.48 | | 40 or without |
| | | | addition |
| SMP-AO-LGG | 7.72 | 60 | |

### Results of Short Chain Fatty Acid Analysis

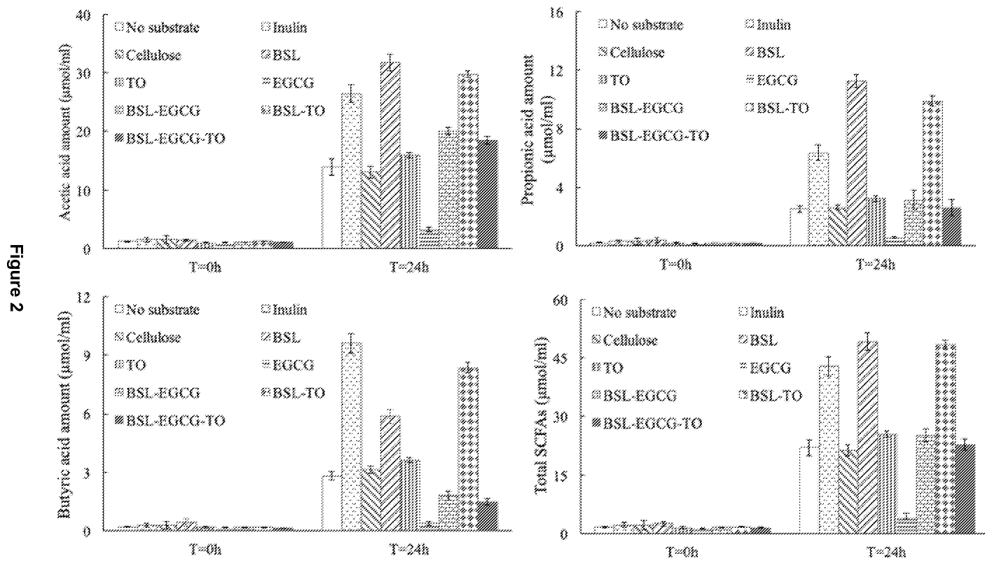

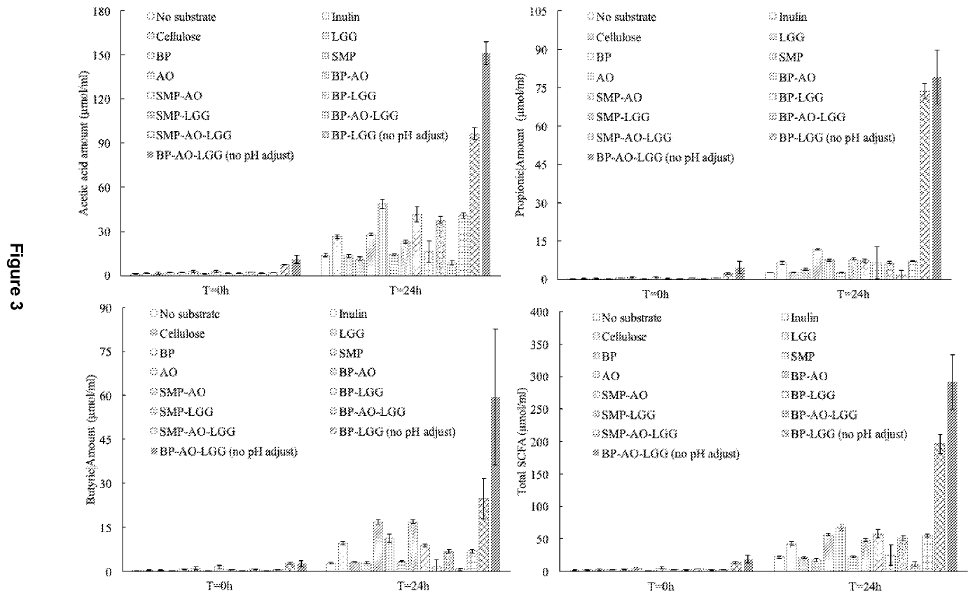

Dietary fibres, proteins and peptides which escape the digestion are metabolized by the microbiota in the colon. SCFA, in particular, acetate, propionate, and butyric, are the most abundant and physiologically important products from gut microbiota fermentation, which act as key sources of energy for colorectal tissues and signalling molecules (Koh et al., 2016). SCFA contribute to gut metabolism and immunity. The concentrations of SCFA production at time 0 hr and the following 24 hrs fermentation of formulations from series 1 and series 2 studies are provided in FIG. 2 and FIG. 3, respectively.

TABLE 5
The pH value in batch fermentation at 0 hr and 24 hrs.

| Substrates | pH at 0 hr | pH at 24 hrs |
|---|---|---|
| Series 1 | | |
| Without substrate | 6.80 ± 0.01 | 6.82 ± 0.04 |
| Inulin | 6.78 ± 0.01 | 4.18 ± 0.04 |
| Cellulose | 6.79 ± 0.03 | 6.44 ± 0.04 |
| BSL | 6.67 ± 0.07 | 5.21 ± 0.01 |
| Tuna oil (TO) | 6.68 ± 0.04 | 6.46 ± 0.05 |
| EGCG | 6.66 ± 0.07 | 6.46 ± 0.05 |
| BSL-EGCG | 6.69 ± 0.03 | 6.05 ± 0.03 |
| BSL-TO | 6.74 ± 0.04 | 5.35 ± 0.05 |
| BSL-EGCG-TO | 6.73 ± 0.04 | 6.09 ± 0.07 |
| Series 2 | | |
| LGG | 6.65 ± 0.07 | 4.53 ± 0.03 |
| BP | 6.77 ± 0.04 | 5.12 ± 0.02 |
| SMP | 6.81 ± 0.03 | 4.11 ± 0.10 |
| Algal oil (AO) | 6.64 ± 0.04 | 5.79 ± 0.13 |
| BP-AO | 6.82 ± 0.02 | 5.15 ± 0.01 |
| SMP-AO | 6.81 ± 0.03 | 4.11 ± 0.12 |
| BP-LGG | 6.47 ± 0.04 | 4.98 ± 0.23 |
| SMP-LGG | 6.75 ± 0.03 | 4.00 ± 0.03 |
| BP-AO-LGG | 6.61 ± 0.05 | 4.91 ± 0.08 |
| SMP-AO-LGG | 6.76 ± 0.02 | 4.12 ± 0.03 |
| BP-LGG without | 6.26 ± 0.06 | 5.21 ± 0.04 |
| pH adjustment | | |
| BP-AO-LGG without | 6.47 ± 0.04 | 5.30 ± 0.12 |
| pH adjustment | | |

FIG. 2 shows that the total and individual SCFA production during fermentation was significantly inhibited by the presence of EGCG. The total SCFA, acetic acid and propionic acid of broccoli stems and leaves (BSL) and BSL-TO formulations were higher than that of positive control inulin whereas inulin had highest butyric acid production. The SCFA production of TO and BSL-EGCG were similar with negative controls.

Series 2 study involved formulations with broccoli puree and LGG. LGG is used for treating intestinal disorders (Krishna and Samak, 2013). LGG has the ability to produce and release propionate in significant levels but is not able to produce either butyrate or acetate (LeBlanc et al., 2017). Accordingly, any butyrate or acetate detected in the fermentation samples as provided in FIG. 3 has resulted from fermentation by the supplemented bacteria in the faecal slurry.

The SCFA study (FIG. 3) demonstrates that fermentation with AO or LGG alone had little or no effect on SCFA production. However, the inclusion of BP or SMP resulted in higher production of acetic acid, butyric acid, propionic acid, and total SCFA in fermentation samples comprising BP-LGG and BP-AO-LGG formulations (both without pH adjustment), SMP-LGG and SMP-AO-LGG compared to the positive control (cellulose). Interestingly, the highest levels of acetic acid, butyric acid, propionic acid, and total SCFA were detected in samples comprising BP-AO-LGG (without pH adjustment).

BP formulated with LGG increased the total SCFA and individual SCFA (acetic acid, butyric acid and propionic acid) produced in comparison with BP alone. The production of these SCFA was further enhanced with the inclusion of AO in the formulation (BP-AO-LGG).

### Example 3

### Effect of Broccoli and Broccoli-Algal Oil Matrices on the Microbiota of the Gut in an In Vitro Colonic Fermentation Model

The samples generated from studies conducted in Example 2 were used to assess the effect of broccoli puree from stems and leaves (BSL, series 1) and broccoli puree from head (BP, series 2), BSL-tuna oil , BSL-EGCG, BSL-EGCG-TO and BP-algal oil (AO), BP-LGG and BP-AO-LGG matrices on the microbiota of the faecal slurry used in the in vitro colonic fermentation.

### Methods

DNA extraction: Aliquots (1 mL) of the fermentation samples were centrifuged at 14000 rpm, 4° C. for 5 min and the supernatants were removed. For EGCG samples, sterile water (1 mL) was added to wash the microbial then remove the supernatant and repeat once. DNA extraction was followed by the PowerMag® Microbiome RNA/DNA Isolation Kit (27500-4-EP; MO BIO Laboratories, Inc., Carlsbad, Calif., USA) optimised for epMotion® platforms with slight modifications. Briefly, 0.8 g of glass beads and 490 μL of pre-warmed PowerMag®Microbiome Lysis Solution were added to faeces. Then the mixtures were homogenised by MagNAlyser at 7000 rpm for 60 s, followed by centrifugation at 10000 rpm for 5 min at 4° C. The supernatant was recovered, and 30 μL of Proteinase K>600 mAU/mL (Qiagen, Hilden, Germany) was added. The sample was heat treated at 70° C. for 10 min. Immediately on completion of the heating step, 110 μL of PowerMag®Inhibitor Removal Solution was added. Samples were then incubated at −20° C. for 5 min, and centrifuged at 14000 rpm for 5 min. The supernatant was placed into a MO BIO 2 mL Deep Well Plate, and 5 μL RNAse (10 mg/mL) was added to each sample. The remaining extraction procedure was determined using the manufacturer's protocol (epMotion-protocol-27500-V2.dws) optimised for epMotion®5075 (Eppendorf AG, Hamburg, Germany). DNA concentrations and purity were measured by Qubit and spectrophotometrically (NanoDrop 1000 spectrophotometer, Thermo Fisher Scientific, Wilmington, Del., USA).

Bacterial population analysis: Aliquots (1 mL) of the fermentation samples were centrifuged at 14000 rpm, 4° C. for 5 min and the supernatants removed. For EGCG samples, sterile water (1 mL) was added to wash the microbial then remove the supernatant and repeat once. DNA extraction was followed by the PowerMag® Microbiome RNA/DNA Isolation Kit (27500-4-EP; MO BIO Laboratories, Inc., Carlsbad, Calif., USA) optimised for epMotion® platforms with slight modifications. Briefly, 0.8 g of glass beads and 490 μL of pre-warmed PowerMag® Microbiome Lysis Solution were added to faeces. Then the mixtures were homogenised by MagNAlyser at 7000 rpm for 60 s, followed by centrifugation at 10000 rpm for 5 min at 4° C. The supernatant was recovered, and 30 μL of Proteinase K>600 mAU/mL (Qiagen, Hilden, Germany) was added. The sample was heat treated at 70° C. for 10 min. Immediately on completion of the heating step, 110 μL of PowerMag® Inhibitor Removal Solution was added. Samples were then incubated at −20° C. for 5 min, and centrifuged at 14000 rpm for 5 min. The supernatant was placed into a MO BIO 2 mL Deep Well Plate, and 5 μL RNAse (10 mg/mL) was added to each sample. The remaining extraction procedure was determined using the manufacturer's protocol (epMotion-protocol-27500-V2.dws) optimised for epMotion® 5075 (Eppendorf AG, Hamburg, Germany). DNA concentrations and purity were measured by Qubit and spectrophotometrically (NanoDrop 1000 spectrophotometer, Thermo Fisher Scientific, Wilmington, Del., USA).

16s rRNA assessment: A broad assessment of microbial population changes was carried out by PCR amplification of the 16S rRNA region of DNA extracted from the ferment samples and the sequencing of the amplified DNA. Sequencing was performed at the Australian Genome Research Facility. In brief, 300 bp sequencing of the V1-V3 region of the 16S rRNA region using an Illumina MiSeq. Paired-ends reads were assembled by aligning the forward and reverse reads using PEAR (version 0.9.5). Primers were identified and trimmed. Trimmed sequences were processed using Quantitative Insights into Microbial Ecology (QIIME 1.8.4), USEARCH (version 8.0.1623), and UPARSE software. Using usearch tools sequences were quality filtered, full length duplicate sequences were removed and sorted by abundance. Singletons or unique reads in the data set were discarded. Sequences were clustered followed by chimera filtered using “rdp_gold” database as reference. To obtain number of reads in each OTU, reads were mapped back to OTUs with a minimum identity of 97%. Taxonomy was assigned using QIIME.

### Results

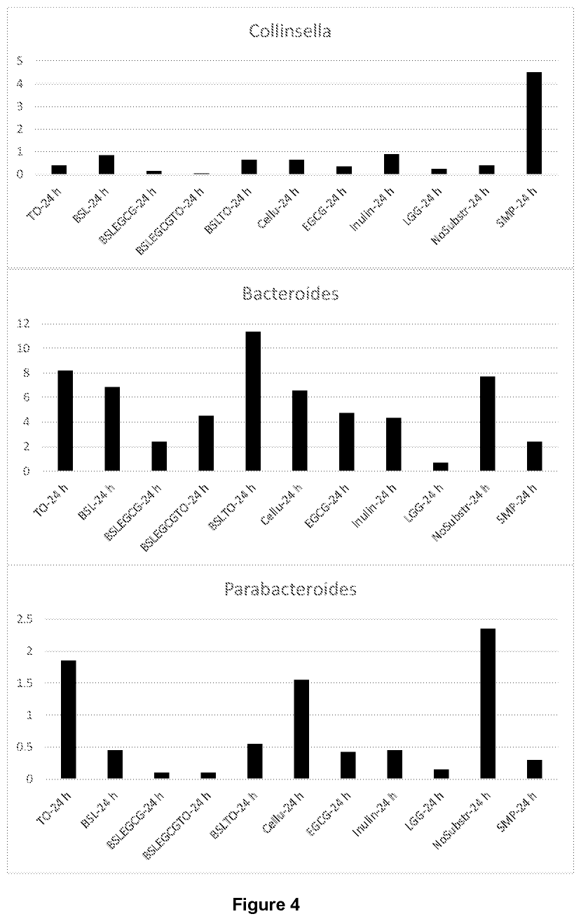

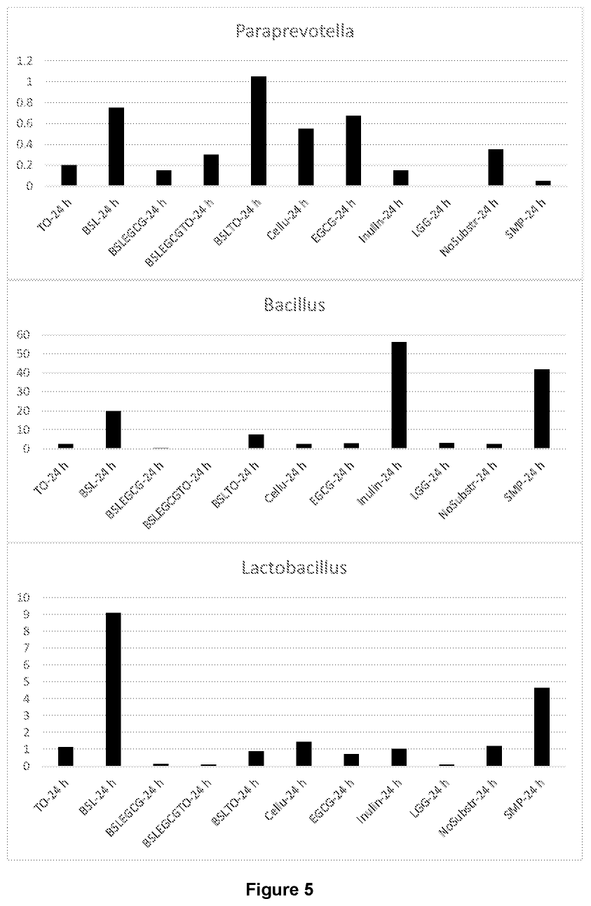

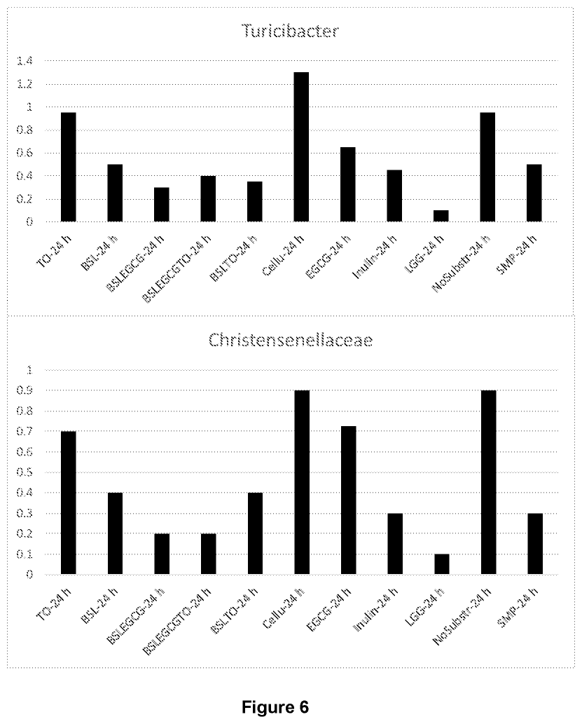

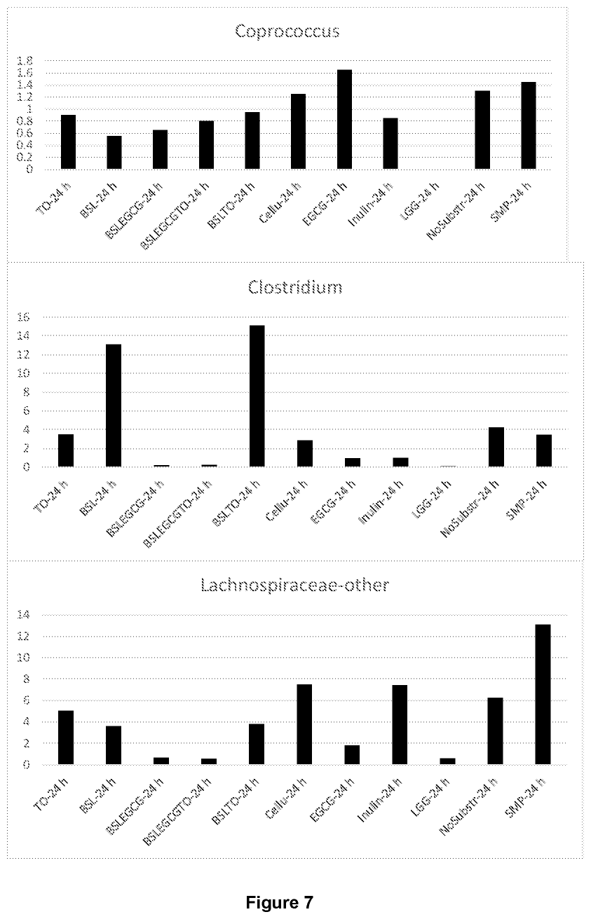

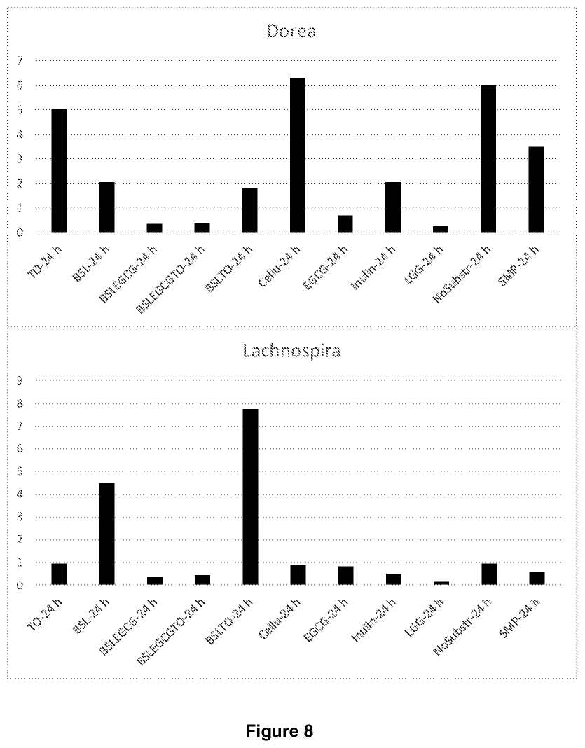

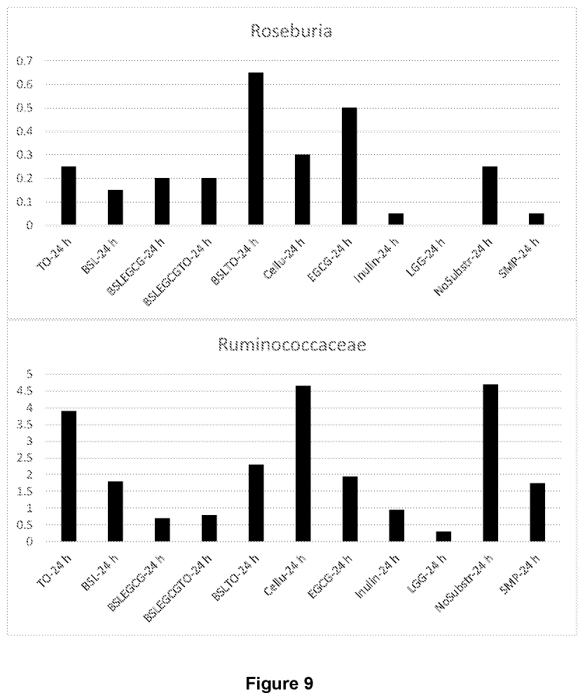

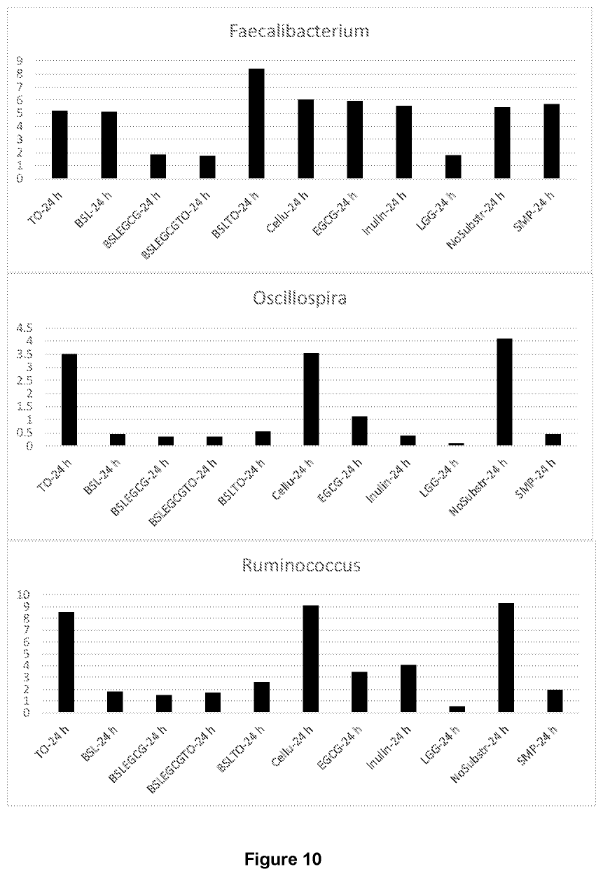

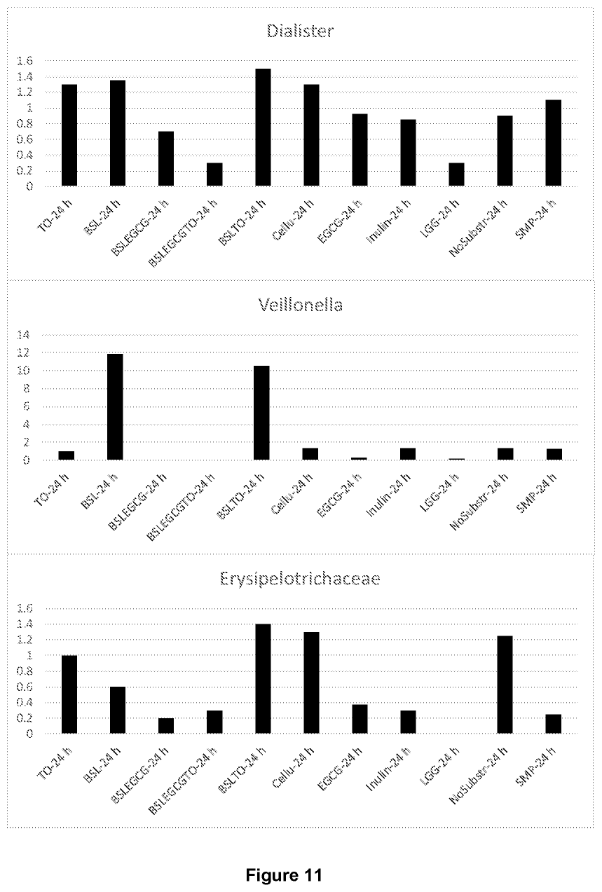

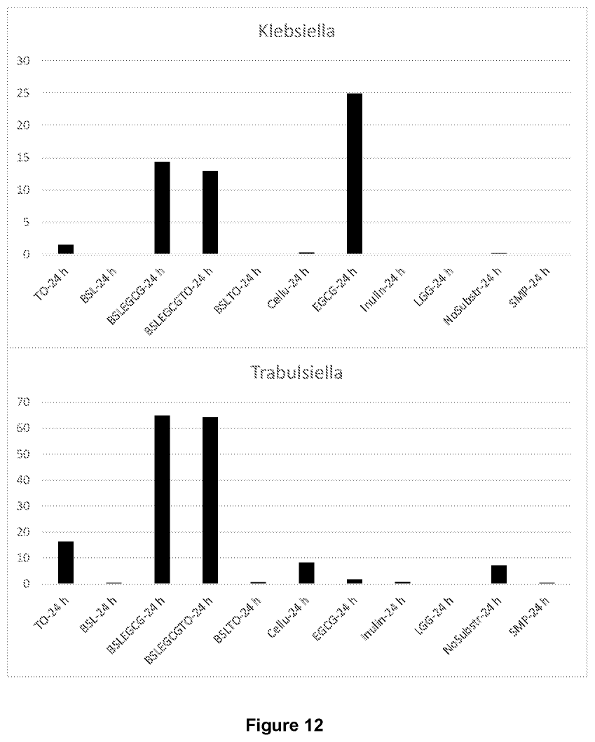

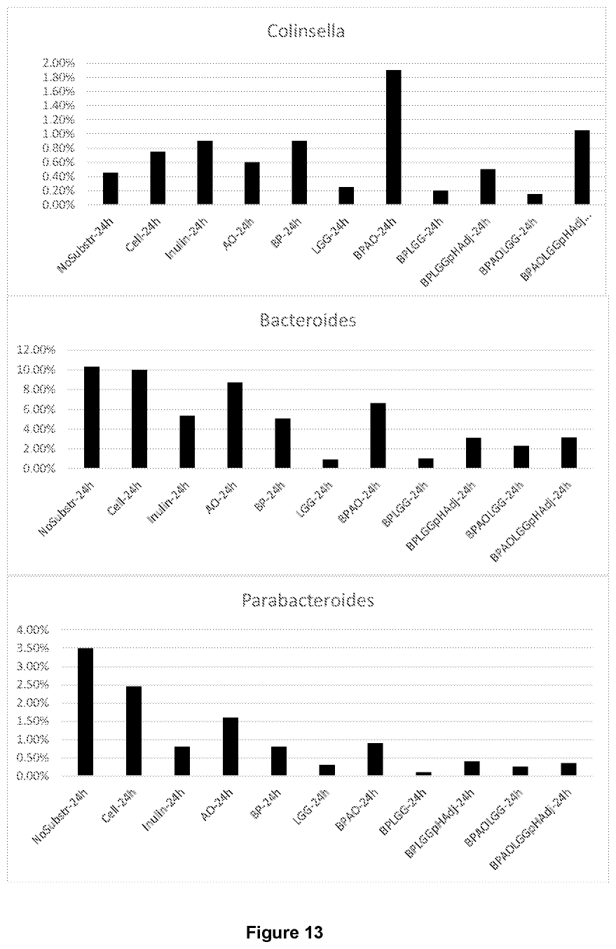

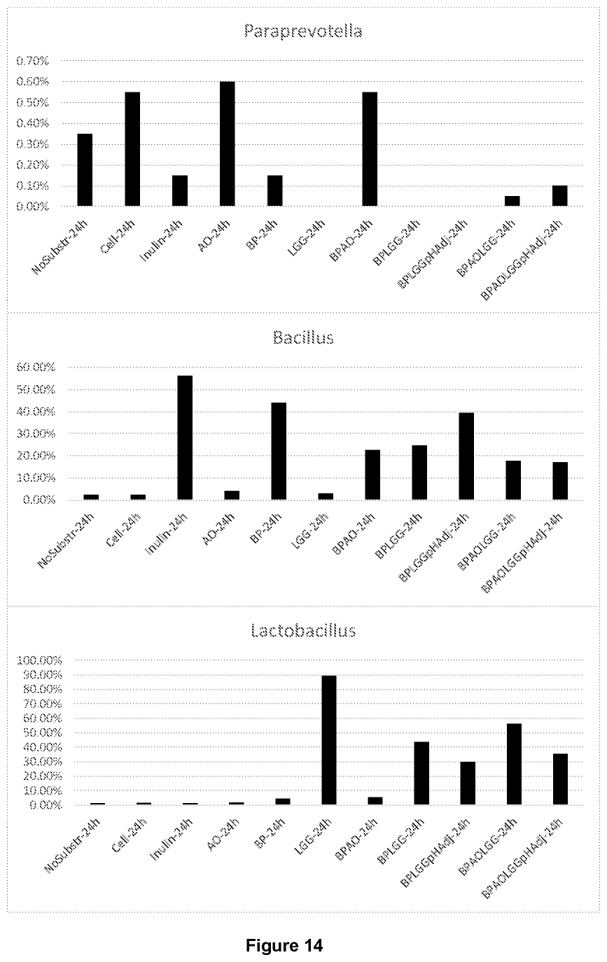

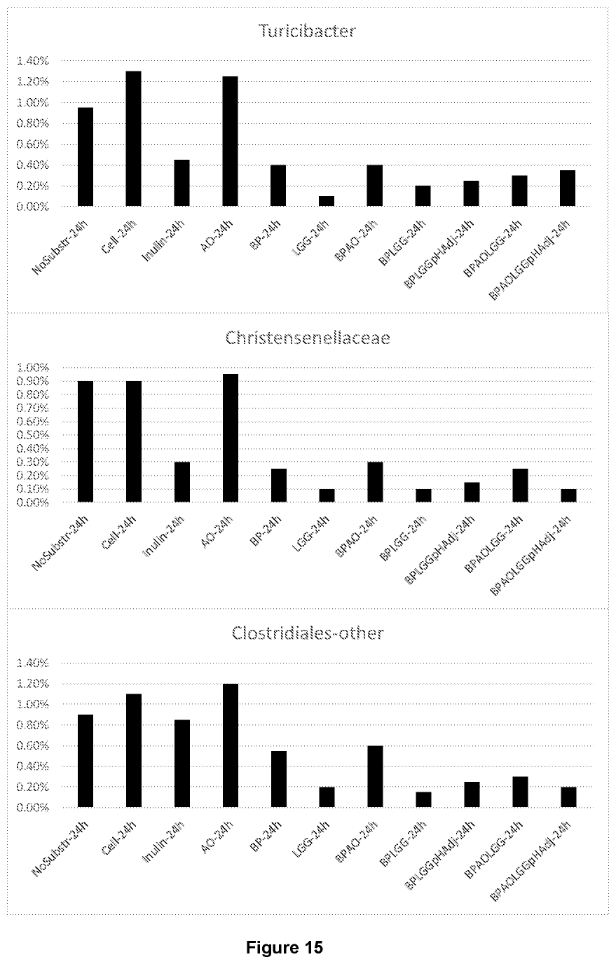

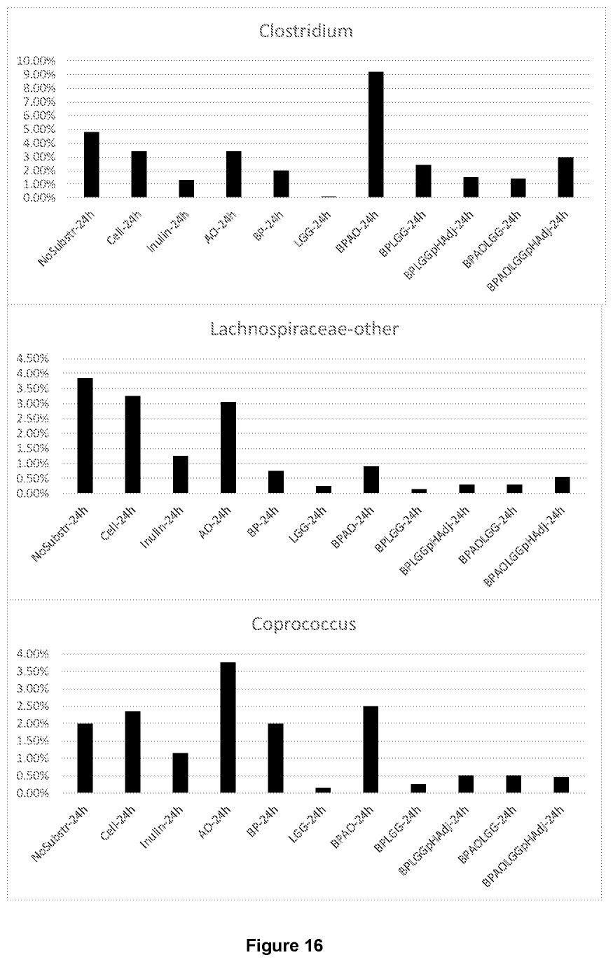

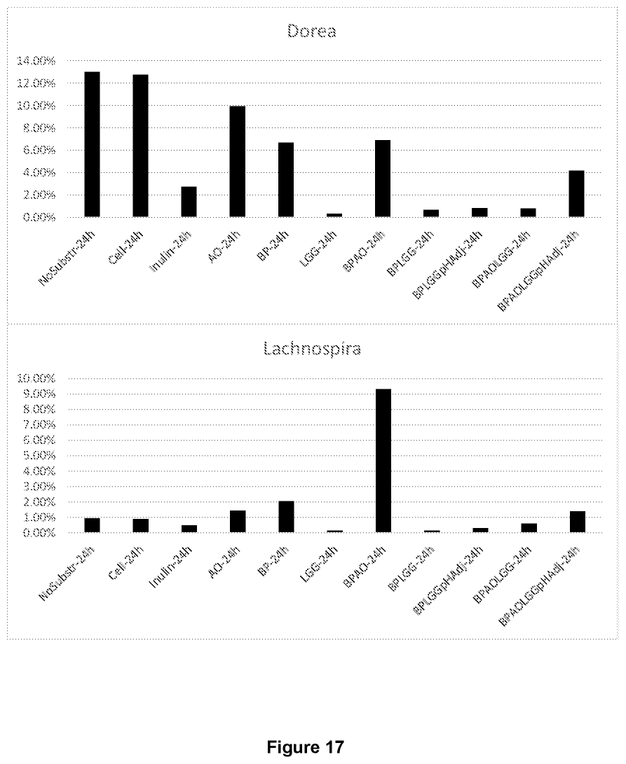

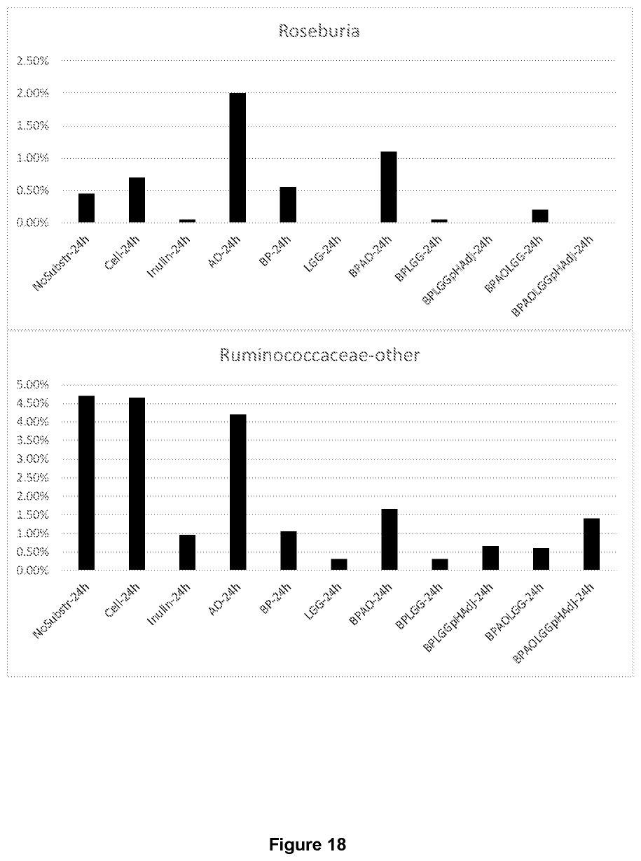

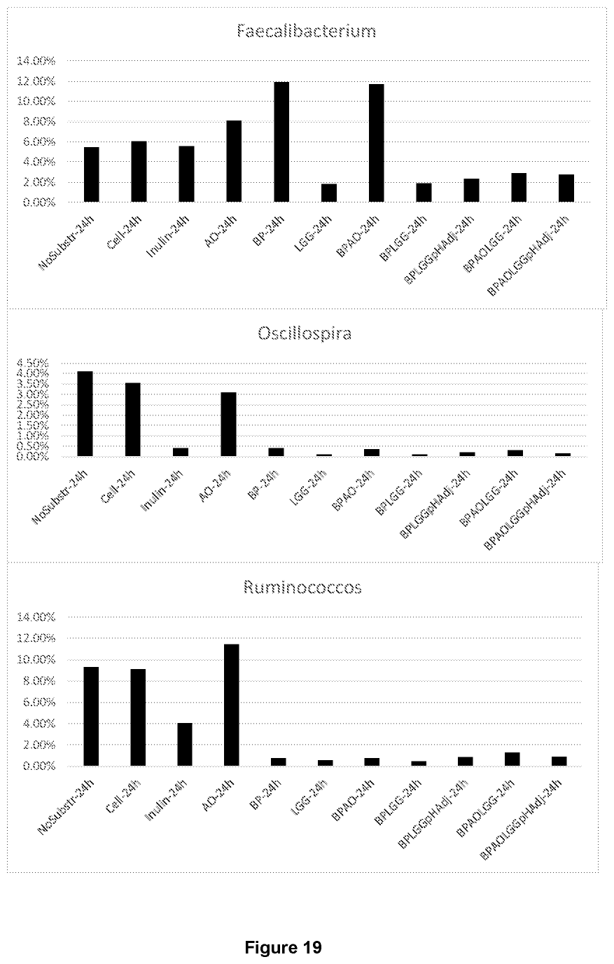

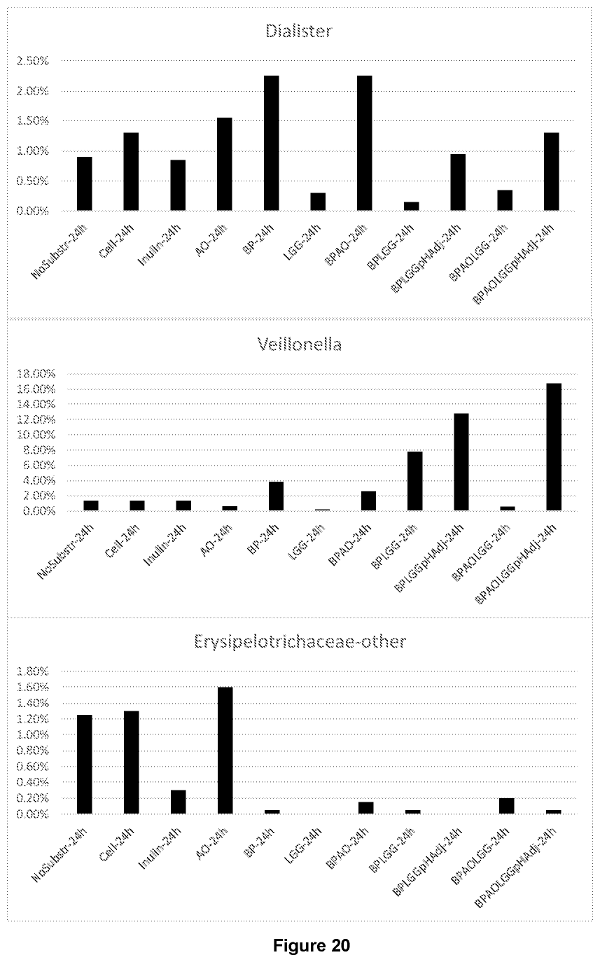

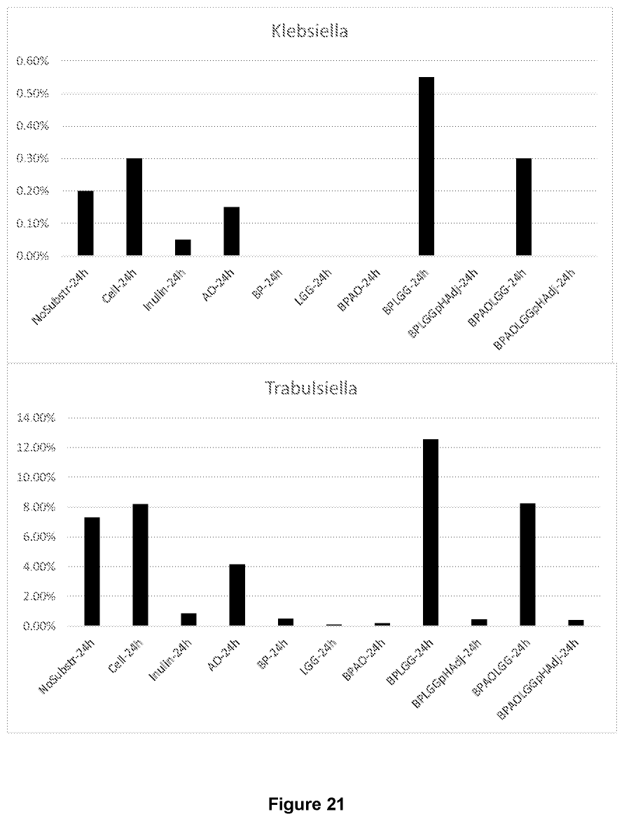

Plots were made showing effects of in vitro fermentation treatments on the % relative abundances of each of the bacteria PCR amplification of the 16S rRNA region, at the genus classification level (Tables 6 and 7 and FIGS. 4 to 21). Only bacteria which were approximately 1% or more of the total bacterial abundance were plotted.

The data in Table 6 represents the relative abundances of bacteria after 24 h fermentation post treatment with one of the following formulations: no substrate, cellulose (cell), Inulin (positive control), Tuna oil (TO), puree made from broccoli stems and leaves (BSL), epigallocatechin gallate (EGCG), BSL-TO, BSL-EGCG, BSL-EGCG-TO, skim milk powder (SMP) and Lactobacillus rhamnosusGG (LGG). The data in Table 7 represents the relative abundances of bacteria after 24 h fermentation post treatment with one of the following formulations: algal oil (AO), broccoli puree (BP), Lactobacillus rhamnosusGG (LGG), broccoli puree+algal oil (BP-AO), broccoli puree+L. rhamnosus LGG (BP-LGG), BP-LGG pH adjusted, broccoli puree+algal oil+L. rhamnosus LGG (BP-AO-LGG) or BP-AO-LGG pH adjusted. The data represent the average of 2 replicates.

Of relevance is that there is significant variation in gut microbiota populations between individuals and consequently the starting microbial composition of the donor stool inoculum which was pooled from several individuals will have an influence on which microbes are present and their abundance.

Each of the key substrates tested (broccoli powder, Lactobacillus LGG probiotic, Tuna oil or algal oil) had some ability to modulate the composition of the bacteria populations following 24 h fermentation when compared to effects of fermentations without substrate or with the poorly fermenting control (cellulose). However, algal oil alone had little impact on the bacterial populations. In contract, the LGG probiotic was given in numbers which resulted in them representing around 90% of the bacteria.

TABLE 6
Effects of formulations (on shifts in gut microflora in-vitro colonic fermentation model.

| | | | | | | | | | | No | |
|---|---|---|---|---|---|---|---|---|---|---|---|
| | TO | BSL | BSLEGCG | BSLEGCGTO | BSLTO | Cellu | EGCG | Inulin | LGG | substrate | SMP |
| Coriobacteriaceae | 0.37 | 2.52 | −0.79 | NA | 2.66 | 1.11 | −0.81 | 1.58 | 1.29 | 0.95 | NA |
| (Collinsella aerofaciens) | | | | | | | | | | | |
| Bacteroidaceae (Bacteroides) | 0.32 | 0.00 | −2.52 | −1.26 | 0.47 | −0.28 | −0.13 | −0.44 | −1.53 | −0.35 | −2.55 |
| Porphyrom onadaceae | 1.89 | 0.58 | −2.45 | −2.00 | 0.66 | 1.63 | 0.27 | 0.21 | 0.00 | 2.10 | −1.40 |
| (Parabacteroides distasonis) | | | | | | | | | | | |
| Paraprevotellaceae | −1.58 | 0.18 | −2.89 | −1.55 | 0.58 | −0.11 | 0.46 | −2.00 | NA | −0.74 | NA |
| (Paraprevotella) | | | | | | | | | | | |
| Bacillaceae (Bacillus coagulans) | −1.30 | 1.78 | −3.72 | −3.32 | 1.00 | −1.09 | −1.94 | 3.60 | 2.94 | −0.92 | 3.12 |
| Lactobacillaceae (Lactobacillus | 1.06 | 3.50 | −2.38 | −2.56 | 0.81 | 1.40 | −0.37 | 0.69 | 0.00 | 1.11 | 2.95 |
| reuteri) | | | | | | | | | | | |
| Lactobacillaceae | −1.18 | −0.27 | NA | NA | NA | −1.58 | NA | 0.79 | 0.16 | −2.04 | −1.66 |
| (Lactobacillus zeae) | | | | | | | | | | | |
| Turicibacteraceae (Turicibacter) | −0.33 | −0.92 | −2.50 | −1.82 | −1.77 | −0.05 | NA | −1.48 | −0.79 | −0.59 | −1.64 |
| Christensenellaceae | −0.72 | −1.38 | −2.64 | −2.81 | −1.63 | −0.74 | NA | −2.11 | −1.58 | −0.63 | −2.14 |
| Clostridiaceae (Clostridium | 1.61 | 3.34 | −2.94 | −2.25 | 3.94 | 1.02 | −0.79 | −0.33 | −1.00 | 1.55 | 1.50 |
| perfringens) | | | | | | | | | | | |
| Lachnospiraceae (Blautia) | −0.21 | −0.65 | −2.28 | −2.68 | −0.60 | 0.19 | −1.39 | 0.00 | −1.00 | −0.08 | 1.33 |
| Lachnospiraceae (Coprococcus) | −1.91 | −2.65 | −2.26 | −1.70 | −1.82 | −1.53 | −0.22 | −2.14 | NA | −1.37 | −1.16 |
| Lachnospiraceae (Dorea | 0.84 | −0.13 | −2.46 | −2.38 | −0.20 | 1.12 | −1.47 | −0.54 | −1.40 | 1.07 | 0.85 |
| formicigenerans) | | | | | | | | | | | |
| Lachnospiraceae (Lachnospira) | −0.75 | 1.29 | −1.85 | −1.42 | 2.41 | −0.21 | −0.28 | −1.22 | −1.08 | −0.27 | −1.63 |
| Lachnospiraceae (Roseburia | −2.64 | −3.50 | −2.85 | −2.52 | −1.29 | −2.27 | −0.11 | NA | NA | −2.22 | NA |
| faecis) | | | | | | | | | | | |
| Ruminococcaceae | 0.22 | −0.97 | −2.44 | −2.32 | −0.63 | 0.35 | −0.57 | −2.00 | −1.32 | 0.66 | −0.75 |
| Ruminococcaceae | −1.55 | −1.94 | −2.88 | −2.87 | −1.35 | −1.19 | −0.81 | −1.51 | −0.51 | −1.38 | −1.57 |
| (Faecalibacterium prausnitzii) | | | | | | | | | | | |
| Ruminococcaceae (Oscillospira) | 1.00 | −2.00 | −2.42 | −2.29 | −1.67 | 1.20 | −0.31 | −2.29 | −1.95 | 1.50 | −1.90 |
| Ruminococcaceae (Ruminococcus) | −0.46 | −2.40 | −2.83 | −3.17 | −2.16 | −0.48 | −1.35 | −1.60 | −2.27 | −0.43 | −2.53 |
| Veillonellaceae (Dialister) | −0.22 | −0.08 | −0.58 | −1.77 | 0.15 | −0.21 | −0.32 | −0.90 | −0.37 | −0.79 | −0.45 |
| Veillonellaceae (Veillonella | 1.79 | 5.30 | NA | NA | 6.72 | 3.25 | −0.58 | 3.25 | NA | 2.96 | 2.68 |
| dispar) | | | | | | | | | | | |
| Erysipelotrichaceae | −0.58 | −0.73 | −2.81 | −2.11 | −0.03 | −0.50 | −1.30 | −2.50 | NA | −0.56 | −2.39 |
| Enterobacteriaceae (Trabulsiella) | 4.24 | −1.08 | 7.18 | 7.03 | 0.24 | 3.45 | 0.58 | 0.15 | −1.58 | 3.08 | −1.38 |

Note:
Results are expressed as Log₂ fold change of percentage abundance of gut microbiota (Taxa identified on the genus and species level) in 24 h fermented samples versus Time 0 samples. Results are presented at the Family level and the predominant genus contributing to the level is indicated in brackets.

TABLE 7
Effects of formulations (Broccoli puree with LAB, algal oil or a combination of LAB
and algal oil) on shifts in gut microflora in-vitro colonic fermentation model.

| | AO | BP | BPAO | BPAOLGG | BPLGG | Cellu | Inulin | LGG |
|---|---|---|---|---|---|---|---|---|
| Coriobacteriaceae | 1.00 | 1.82 | 2.56 | 0.53 | 0.33 | 1.11 | 1.58 | 1.29 |
| (Collinsella aerofaciens) | | | | | | | | |
| Bacteioidaceae | 0.10 | −1.33 | −0.49 | −1.48 | −1.66 | −0.28 | −0.44 | −1.53 |
| (Bacteroides) | | | | | | | | |
| Porphyromonadaceae | 0.95 | 0.13 | 0.37 | −1.16 | −0.40 | 1.63 | 0.21 | 0.00 |
| (Parabacteroides | | | | | | | | |
| distasonis) | | | | | | | | |
| Paraprevotellaceae | 0.00 | −2.75 | −0.94 | −1.58 | NA | −0.11 | −2.00 | NA |
| (Paraprevotella) | | | | | | | | |
| Bacillaceae | −0.50 | 3.51 | 2.55 | 3.11 | 4.32 | −1.09 | 3.60 | 2.94 |
| (Bacillus coagulans) | | | | | | | | |
| Lactobacillaceae | 0.53 | 3.23 | 3.28 | 3.01 | 3.85 | 1.40 | 0.69 | 0.00 |
| (Lactobacillus reuteri) | | | | | | | | |
| Lactobacillaceae | 0.66 | 1.95 | −0.50 | 0.06 | −0.72 | −1.58 | 0.79 | 0.16 |
| (Lactobacillus zeae) | | | | | | | | |
| Turicibacteraceae | −0.27 | −1.75 | −1.63 | −1.22 | −1.35 | −0.05 | −1.48 | −0.79 |
| (Turicibacter) | | | | | | | | |
| Christensenellaceae | −0.45 | −2.41 | −1.94 | −2.11 | −1.79 | −0.74 | −2.11 | −1.58 |
| Clostridiaceae | 0.79 | −0.10 | 2.71 | 1.37 | 1.93 | 1.02 | −0.33 | −1.00 |
| (Clostridium perfringens) | | | | | | | | |
| Lachnospiraceae | −0.21 | −2.63 | −2.36 | −1.81 | −2.37 | 0.19 | 0.00 | −1.00 |
| (Blautia) | | | | | | | | |
| Lachnospiraceae | −0.66 | −0.98 | −0.95 | −2.73 | −2.42 | −1.53 | −2.14 | NA |
| (Coprococcus) | | | | | | | | |
| Lachnospiraceae | 0.55 | −0.63 | −0.56 | −1.12 | −1.28 | 1.12 | −0.54 | −1.40 |
| (Dorea | | | | | | | | |
| formicigenerans) | | | | | | | | |
| Lachnospiraceae | 0.29 | 0.69 | 2.46 | 0.18 | −1.36 | −0.21 | −1.22 | −1.08 |
| (Lachnospira) | | | | | | | | |
| Lachnospiraceae | −1.23 | −1.50 | −0.42 | −2.89 | NA | −2.27 | NA | NA |
| (Roseburia faecis) | | | | | | | | |
| Ruminococcaceae | 0.23 | −1.63 | −1.24 | −0.88 | −1.51 | 0.35 | −2.00 | −1.32 |
| Ruminococcaceae | −0.85 | −0.42 | −0.44 | −1.59 | −1.72 | −1.19 | −1.51 | −0.51 |
| (Faecalibacterium | | | | | | | | |
| prausnitzii) | | | | | | | | |
| Ruminococcaceae | 0.67 | −2.17 | −2.40 | −1.83 | −2.47 | 1.20 | −2.29 | −1.95 |
| (Oscillospira) | | | | | | | | |
| Ruminococcaceae | −0.21 | −3.98 | −4.01 | −2.33 | −2.75 | −0.48 | −1.60 | −2.27 |
| (Ruminococcus) | | | | | | | | |
| Veillonellaceae | −0.10 | 0.45 | 0.73 | −0.39 | −0.57 | −0.21 | −0.90 | −0.37 |
| (Dialister) | | | | | | | | |
| Veillonellaceae | 2.00 | 4.77 | 4.13 | 4.73 | 6.98 | 3.25 | 3.25 | NA |
| (Veillonella dispar) | | | | | | | | |
| Erysipelotrichaceae | −0.31 | NA | −3.18 | −2.48 | −3.58 | −0.50 | −2.50 | NA |
| Enterobacteriaceae | 1.53 | NA | −1.70 | 2.25 | 3.05 | 3.45 | 0.15 | −1.58 |
| (Trabulsiella) | | | | | | | | |

| | No | | | | |
|---|---|---|---|---|---|
| | substrate | SMP | SMPAO | SMPAOLGG | SMPLGG |
| Coriobacteriaceae | 0.95 | NA | 3.41 | 3.44 | 0.00 |
| (Collinsella aerofaciens) | | | | | |
| Bacteioidaceae | −0.35 | −2.55 | −1.57 | −1.28 | 0.61 |
| (Bacteroides) | | | | | |
| Porphyromonadaceae | 2.10 | −1.40 | −0.53 | −0.21 | 0.50 |
| (Parabacteroides | | | | | |
| distasonis) | | | | | |
| Paraprevotellaceae | −0.74 | NA | −2.49 | NA | NA |
| (Paraprevotella) | | | | | |
| Bacillaceae | −0.92 | 3.12 | 3.18 | 4.15 | −0.33 |
| (Bacillus coagulans) | | | | | |
| Lactobacillaceae | 1.11 | 2.95 | 2.88 | 0.95 | 0.00 |
| (Lactobacillus reuteri) | | | | | |
| Lactobacillaceae | −2.04 | −1.66 | 0.48 | −0.67 | 0.12 |
| (Lactobacillus zeae) | | | | | |
| Turicibacteraceae | −0.59 | −1.64 | −1.43 | −0.16 | 0.50 |
| (Turicibacter) | | | | | |
| Christensenellaceae | −0.63 | −2.14 | −2.14 | −0.66 | 0.79 |
| Clostridiaceae | 1.55 | 1.50 | 1.62 | 1.15 | −0.21 |
| (Clostridium perfringens) | | | | | |
| Lachnospiraceae | −0.08 | 1.33 | 0.46 | 1.24 | −0.08 |
| (Blautia) | | | | | |
| Lachnospiraceae | −1.37 | −1.16 | −2.13 | −0.67 | 0.61 |
| (Coprococcus) | | | | | |
| Lachnospiraceae | 1.07 | 0.85 | −0.33 | 0.61 | 0.23 |
| (Dorea | | | | | |
| formicigenerans) | | | | | |
| Lachnospiraceae | −0.27 | −1.63 | −1.07 | 0.00 | 0.21 |
| (Lachnospira) | | | | | |
| Lachnospiraceae | −2.22 | NA | −3.25 | NA | NA |
| (Roseburia faecis) | | | | | |
| Ruminococcaceae | 0.66 | −0.75 | −1.29 | −0.21 | 0.16 |
| Ruminococcaceae | −1.38 | −1.57 | −1.51 | −0.23 | 0.37 |
| (Faecalibacterium | | | | | |
| prausnitzii) | | | | | |
| Ruminococcaceae | 1.50 | −1.90 | −1.75 | −0.61 | 0.21 |
| (Oscillospira) | | | | | |
| Ruminococcaceae | −0.43 | −2.53 | −2.43 | −1.05 | 0.43 |
| (Ruminococcus) | | | | | |
| Veillonellaceae | −0.79 | −0.45 | −0.29 | 0.40 | 0.26 |
| (Dialister) | | | | | |
| Veillonellaceae | 2.96 | 2.68 | 2.90 | 3.23 | −0.08 |
| (Veillonella dispar) | | | | | |
| Erysipelotrichaceae | −0.56 | −2.39 | −3.29 | NA | 0.50 |
| Enterobacteriaceae | 3.08 | −1.38 | −0.58 | 0.96 | 2.50 |
| (Trabulsiella) | | | | | |

Note:
Note: Results are expressed as Log2 fold change of percentage abundance of gut microbiota (Taxa identified on the genus and species level) in 24 h fermented samples versus Time 0 samples. Results are presented at the Family level and the predominant genus contributing to the level is indicated in brackets.

Broccoli stems and leaves powder (BSL) with or without tuna oil (TO) increased the abundance of: Colinsella, Bacillus, Lactobacillus, Lachnospira, and Veillonellaceae (Veillonella dispar). When BSL was combined with EGCG, the powder decreased all these abundance.

Of relevance is that there is significant variation in gut microbiota populations between individuals and consequently the starting microbial composition of the donor stool inoculum which was pooled from several individuals will have an influence on which microbes are present and their abundance.

Broccoli powder (BP) increased the abundance of: Colinsella (when combined with Algal oil), Bacillus, Lactobacillus, Lachnospira (especially in combination with algal oil), Faecalibacterium, Dialister, and Veillonella.

Broccoli powder (BP) decreased the abundance of: Bacteroides, Parabacteroides, Paraprevotella, Turicibacter, Christensenellaceae, some members of Family Clostridiales, Clostridium, some members of Family Lachnospiraceae, Dorea, Roseburia, some members of the Family Ruminococcaceae, Oscillospira, Ruminococcus, some members of the Family Erysipelotrichaceae, Klebsiella and Trabulsiella. Algal oil increased the abundance of: Coprococcus and Roseburia, Algal oil decreased the abundance of: Parabacteroides, Dorea, (both borderline decreases) LGG increased the abundance of: Lactobacillus. LGG decreased the abundance of: All other microbes whose populations were above around 1% of the total.

Some beneficial effects of broccoli puree were observed. Lactobacillus are a genus of bacteria which are commonly used as probiotics. Foods which increase this genus in the gut are generally regarded as prebiotics. Hence, Lactobacillus is regarded as beneficial. In this study, in vitro fermentations containing BP increased the abundance of this genus of bacteria compared to controls, including the positive control inulin which is a recognised prebiotic. Therefore, the increase in Lactobacillus induced by fermentation with broccoli powder suggests broccoli powder would probably act as a prebiotic if consumed.

Other changes induced by broccoli substrates (BSL or BP) could also be beneficial. The genus Colinsella has been implicated in reduction of bloating in irritable bowel syndrome and numbers were increased by the broccoli substrates treatment. Also, the broccoli substrates increased numbers of Lachnospira, Veillonella and Faecalibacterium. Low numbers of microbes in these 3 genera have been linked with asthma in young infants. Of note is the amplification of impacts on Colinsella and Lachnospira when broccoli puree was given with algal oil. Clostridium was also significantly increased when the broccoli puree and algal oil were used in combination.

Faecalibacterium are key producers of a short chain fatty acid known as butyrate and the microbe has also been demonstrated to have some anti-inflammatory effects. Butyrate has multiple beneficial effects within the large bowel. Hence the increase in Faecalibacterium by broccoli substrates can also be beneficial from the perspective of its ability to facilitate butyrate production.

The reduction in numbers of Bacteroides may also be beneficial as many species from this genus are considered pathogenic.

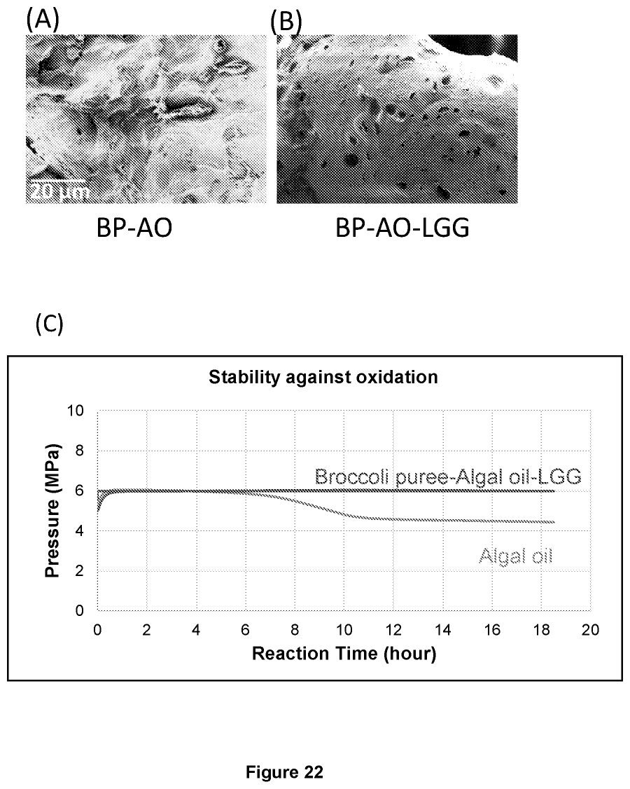

Micrographs (FIG. 22) of broccoli puree made from head with algal oil (BP-AO) and the sample with LGG (BP-AO-LGG) show the microstructures of the encapsulation systems.

### Example 4

### Survival of Lactic Acid Bacteria (LAB) in Broccoli and Broccoli-Algal Oil Matrices During Freeze Drying

### Methods

Preparation of powders: LGG was used as the model probiotic bacteria that was carried in the broccoli-based matrices. LGG culture preparation (Chr.Hansen, Horsholm, Denmark freeze-dried culture) was added into the broccoli-based matrices. The powders were formulated to contained 7.7 E+10 colony forming unity (CFU)/g powder. The following powder formulations were prepared using BP: (a) BP-LGG powder: 90% BP and 10% LGG culture preparation (dry basis) and (b) BP-algal oil-LGG powder: 80% BP, 10% algal oil and 10% LGG culture preparation (dry basis). Briefly, BP was mixed with water at a ratio of 2:1, colloid-milled 10 times to reduce particle size for encapsulation. BP was mixed) for 30 s to prepare BP-LGG mixtures and freeze dried to prepare the BP-LGG powder. For the BP-algal oil (AO)-LGG powder, the BP was diluted to 5% total solids, mixed with algal oil for 5 min at maximum speed using a Silverson emulsifier-mixer, LGG culture preparation was added, and the mixture was then freeze dried.

Enumeration of bacterial viability: For a viable bacteria count, each sample was diluted in fresh peptone saline diluent in a series of up to 10⁻⁹to determine the number of viable cells. A 100 μL aliquot of the diluted sample was inoculated onto MRS agar and incubated overnight at 37° C. Each viable unit (cell) grown as a colony was counted as a colony forming unit (CFU). The number of CFU per mL of the sample suspension is compared to the number of cells in the sample. The viable cell counts are transformed into log10 value.

Loss of viable LGG cells due to digestion or storage were determined against the number of LGG cells in the formulations.

Viability after freeze drying: De Man, Rogosa and Sharpe agar, MRS agar and peptone saline diluent (Peptone 1.0 g/L, Sodium chloride 8.5 g/L) was used for all enumerations of LGG from the samples. The equivalent quantity of freeze-dried powders required for viable cell counts was determined based on the total volume of samples that had been used for freeze-drying and the amount of powder gained after freeze-drying. Freeze-dried powder (1 g) was re-suspended in 10 mL of MRS broth by vortexing for approximately 1 min. This culture was then serially diluted and followed by spread-plating on fresh agar plates. All plates were incubated anaerobically at 37° C. for 2 days.

### Results

Before freeze drying, the powders had 7.7 E+10 colony forming unity (CFU)/g powder. After freeze-drying, the viable count cells in the BP-LGG powder (90% broccoli puree+10% LGG) was 1.97 E+10 CFU/g powder and that of BP-AO-LGG (80% broccoli puree+10% algal oil+10% LGG) was 8.15E+09 CFU/g powder. This represents a loss of less than 1 log CFU/g.

### Example 5

### Survival of Lactic Acid Bacteria (LAB) in Broccoli and Broccoli-Algal Oil Matrices During Storage

### Methods

Freeze-dried broccoli powders (BP-LGG and BP-AO-LGG) and dried LGG culture preparation without the broccoli matrix (formulations as provided in Example 1) were stored in open containers inside a desiccator at 25° C. over saturated salt solutions of magnesium chloride (MgCl₂) or magnesium nitrate (Mg (NO₃)₂) to provide atmospheres with relative humidity, RH of 32% (0.30 a_{w}) and 52% (0.50 a_{w}), respectively. The viable counts were obtained prior to and after storage.

### Results

The initial viable counts of the samples and the viable counts after 1-week storage of the powders at 25° C. at different water activities (0.3 and 0.5 a_{w}) are given in Table 8. There was a higher loss of viable counts when probiotic bacteria were not with broccoli-based matrices under accelerated conditions of storage 25° C. (0.30 and 0.50 a_{w}).

TABLE 8
Survival of LGG cells without broccoli matrices and lactic acid bacteria*
with broccoli-based matrices after storage at 25° C. at 32% RH (0.3 a_{w})
and 52% RH (0.5 a_{w}) after 1 week and 12 week storage.

| | | Loss of Lactic acid bacteria count | | | |
|---|---|---|---|---|---|
| | | compared to Freeze-dried powder | | | |
| | Viable | after storage at 25° C. | | | |
| | count/g | (log₁₀ transformed value) | | | |
| | powder | After 1-week | | After 12-week | |
| | Before | storage | | storage | |
| | storage | 0.30 a_{w} | 0.50 a_{w} | 0.30 a_{w} | 0.50 a_{w} |
| LGG | 12.23 ± 0.3 | 1.8 | 1.7 | 5.56 | 6.20 |
| 90% broccoli | 9.37 ± 0.5 | 0.1 | No | 3.68 | 3.68 |
| puree + | | | Loss | | |
| 10% LGG | | | | | |
| 80% broccoli | 9.61 ± 0.05 | No | 1.3 | 4.07 | 4.72 |
| puree + 10% | | Loss | | | |
| algal oil + | | | | | |
| 10% LGG | | | | | |

Note*:
The LAB count in broccoli puree prior to the addition of LGG was 1.70E+04, which was due to the presence of autochthonous LAB.

### Example 6

### Survival of Lactic Acid Bacteria Carried by Broccoli and Broccoli-Algal Oil Matrices During In-Vitro Digestion and Survival of Lactic Acid Bacteria During In-Vitro Digestion

### Materials

Simulated gastric fluid (SGF): SGF was prepared on the day of conducting the in-vitro digestion experiments by dissolving sodium chloride (2.0±0.01 g) in 800 mL of Milli-Q water and adjusted to pH 1.2, using 37% in water (w/v). Pepsin (1.6±0.01 g) was added, and the solution was mixed for 30 min, made up to 1000 mL with Milli-Q water, purged with argon, and stored at 4° C. until required.

Simulated intestinal fluid (SIF): SIF was prepared on the day of conducting the in-vitro digestion experiments. Anhydrous potassium dihydrogen phosphate (17±0.01 g) was dissolved in 750 mL of Milli-Q water followed by the addition of 192.5 mL of 0.2M NaOH. The solution was adjusted to pH 6.8, using 1M NaOH, and 3.15±0.01 g of pancreatin added. The volume of the solution was made up to 1000 mL with Milli-Q water. Calcium chloride (CaCl₂) at 0.05 M was prepared.

### Methods

The following powders BP-LGG powder: 90% BP and 10% LGG culture preparation (dry basis); BP-AO-LGG powder: 80% BP, 10% algal oil and 10% LGG culture preparation (dry basis) and LGG culture preparation were used for assessment of viability after sequential exposure to simulated gastric fluid (SGF) and simulated intestinal fluids (SIF).

The SGF solution (pH adjusted to 1.2 with HCl) comprised sodium chloride (2 g) pepsin (1.6 g) made up to 1000 mL with Milli-Q water. The SIF (pH adjusted to 6.8 using NaOH) contained anhydrous potassium dihydrogen phosphate (17 g), pancreatin (3.15 g) made up to1000 mL with Milli-Q water. For sequential exposure to (SGF+SIF), the freeze dried powder (0.2 g in 9.8 g of deionized water) was added to SGF solution (12.5 mL) and incubated in a shaking water bath (37° C. for 2 hrs at 100 rpm). A blanket of argon was applied to all solutions prior to tightly sealing the glassware. The SGF-digested sample was adjusted to pH 6.8 with 1M NaOH, combined with 10 mL of SIF solution and incubated for 20 min prior to addition of CaCl₂ (0.05 M, 2.5 mL) and incubation for a further 2 hrs and 40 min. The survival or viability of the LGG cells with/without broccoli matrices in simulated digestive fluids (SGF, and SIF) was performed.

### Results

Simulated in vitro digestion of the samples containing LGG was performed by sequential exposure of samples to simulated gastric and intestinal fluids (SGF+SIF). Survival of LGG cells in the in vitro digested samples were plate counted. The initial LGG counts of the LGG culture preparation (without the broccoli matrix) before and after (SGF+SIF), and the total LAB counts of LGG-loaded broccoli matrices before and after (SGF+SIF) are given in Table 9. Probiotic survival was higher in the BP alone matrix (90% of BP) compared to LAB count in the BP+AO matrix (80% BP) and LGG cells without the matrix.

TABLE 9
Survival of LGG cells without the broccoli matrix and survival
of total LAB* in LGG-loaded broccoli matrices upon sequential
exposure to simulated gastric fluid (SGF) for 2 hrs and
simulated intestinal fluid (SIF) for 3 hrs.

| | | Viable count/g | | Loss of LAB |
|---|---|---|---|---|
| | | powder (log₁₀ | | count (log₁₀ |
| | | transformed value) | | transformed value) |
| | | Before | After | After |
| | | in-vitro | in-vitro | in-vitro |
| | Sample | digestion | digestion | digestion |
| Trial | LGG | 12.2 ± 0.3 | 4.0 ± 0.1 | 8.2 |
| 1* | 90% broccoli | 9.4 ± 0.5 | 5.5 ± 0.1 | 3.9 |
| | puree + | | | |
| | 10% LGG | | | |
| | 80% broccoli | 9.6 ± 0.05 | 5.2 ± 0.2 | 4.4 |
| | puree + 10% | | | |
| | algal oil + | | | |
| | 10% LGG | | | |
| Trial | LGG | 11.47 ± 0.43 | 4.92 ± 0.11 | 6.6 |
| 2 | 90% broccoli | 6.77 ± 0.10 | 5.31 ± 0.12 | 1.5 |
| | puree + | | | |
| | 10% LGG | | | |
| | 80% broccoli | 8.01 ± 0.01 | 4.79 ± 0.12 | 3.2 |
| | puree + 10% | | | |
| | algal oil + | | | |
| | 10% LGG | | | |

Note*:
The LAB count in broccoli puree prior to the addition of LGG was 1.70E+04, which was due to the presence of autochthonous LAB in Trial 1. In Trial 2, sterilised puree was used.

The results of Table 9 demonstrate that there was a significant (p<0.05) reduction of LGG cells of BP comprising formulations (˜4 log/g sample) upon sequential exposure to SGF and SIF in all BP comprising formulations. The survival of LGG cells of BP-LGG formulation was higher compared to BP-AO-LGG formulation and dried LGG cells upon sequential exposure of SGF and SIF. The results indicate that a higher percentage of BP in the formulation helps to improve the survival of LGG in simulated digestive fluids.

### Example 7

### Oxidative Stability of Algal Oil in Broccoli-Algal Oil-LGG Delivery Systems

### Methods

The broccoli-algal oil-LGG (BP-AO-LGG) powder: 80% BP, 10% algal oil and 10% LGG culture preparation (dry basis) prepared as described in Example 3 above and algal oil were stored in a screw-capped, amber coated container with headspace air for 2 weeks at 40° C. The fatty acid composition of the algal oil and broccoli powder (BP-AO-LGG: powder containing 80% broccoli puree+10% algal oil+10% LGG) and the % remaining docosahexaenoic acid (DHA) in the stored samples (40° C. for 2 weeks) were determined. Fatty acid composition was determined by gas chromatography (GC) after preparation of fatty acid methyl esters (FAME). A mixture of the powder (10 mg) and internal standards (0.75 mg of 17:0 triheptadecanoin, triacylglycerol (TAG) in toluene) were resuspended in 0.9 mL 1N methanolic HCl and 0.1 mL of dichloromethane in an argon-flushed 2 mL GC vial, For algal oil, 5.0±0.01 mg was mixed with the internal standards (0.5 mg of 17:0 triheptadecanoin, TAG in toluene). The mixture was subsequently incubated in a water bath shaker (100 rpm) at 80 ° C. for 2 hrs. FAME were extracted with 0.3 mL hexane. The FAME solution (1 μL) was injected at a split ratio of 1:40 into a GC column (BPX 70 fused silica column, 30 m, 0.25 mm id and 0.25 lm films, SGE, Australia), installed in a model 7890A GC system equipped with a model 7693 autosampler (Agilent Technologies Australia Pty Ltd., Mulgrave, Victoria 3170, Australia). The GC column temperature was increased from 60 to 170° C. at a rate of 20° C./min, then to 192° C. at a rate of 1° C./min and finally to 220° ° at 20° C./min. The injector and detector (FID) were held at 220 and 250° C., respectively. Agilent Chemstation software [B.04.02 SP2 (256)] was used to integrate GC peak areas.

### Results

The selected polyunsaturated fatty acid contents of the algal oil and the BP-AO-LGG powder are shown in Table 10. The algal oil had 44.9% DHA. DHA comprised 34.3% of the fatty acids in the BP-AO-LGG powder. When algal oil is added the broccoli matrix with LGG, the fatty acid profile is a combination of fatty acids from all oil components in the sample. In the case of BP-AO-LGG powder, it would include a contribution of lipids present in the puree (i.e. oil in broccoli plus lipids from bacteria present in the broccoli puree).

The DHA content and calculated remaining amounts (%) of the content of BP-AO-LGG powder compared to the algal oil are shown in Table 11. After 2 weeks storage at 40° C., the remaining % of DHA in the stored algal oil samples decreased to 29.1% where that in the BP-AO-LGG samples were 84.1%. The AO within the BP-AO-LGG matrix was protected against oxidation compared to when the neat oil was stored.

After 12 months storage at 25° C., the remaining % of DHA in the BP-AO-LGG samples was 62.8%. It was noticed that the stored neat algal oil samples had deteriorated significantly and formed a gel and FMAE on that sample was not performed. However, AO within the BP-AO-LGG matrix was protected against oxidation.

TABLE 10
Selected omega-3 polyunsaturated fatty acid contents of
the algal oil and broccoli powder evaluated in the study.

| | Before storage | | Stored at 40° C. | | Stored at 25° C. | |
|---|---|---|---|---|---|---|
| | (Time 0) | | for 2 weeks | | for 12 months | |
| | BP- | | BP- | | BP- | |
| Polyunsaturated | AO- | Algal | AO- | Algal | AO- | Algal |
| fatty acids | LGG | oil | LGG | oil | LGG | oil |
| C18:3w6, | 0.5 | 1.0 | 0.4 | 0.5 | 0.2 | NA |
| gamma-Linolenic | | | | | | |
| acid | | | | | | |
| C18:3w3, alpha- | 11.5 | 1.1 | 9.6 | 0.7 | 9.2 | NA |
| Linolenic acid | | | | | | |
| C20:5w3, | 1.3 | 14.1 | 1.1 | 5.0 | 1.0 | NA |
| Eicosapentaenoic | | | | | | |
| acid | | | | | | |
| C22:6w3, | 39.4 | 436.7 | 33.1 | 127.0 | 28.9 | NA |
| Docosahexaenoic | | | | | | |
| Acid | | | | | | |

Note*:
The BP-AO-LGG powder contains 10% algal oil. Lipids in broccoli puree (including that of bacteria in the puree) contributes to the fatty acid content of the BP-AO-LGG powder.

TABLE 11
Stability of docosahexaenoic acid (DHA) in broccoli-algal oil-LGG powder compared
to algal oil (before and after the storage at 40° C. for 2 weeks).

| | DHA content at | | Remaining | DHA content at | | Remaining |
|---|---|---|---|---|---|---|
| | 40° C. for | | DHA content | 25° C. for | | DHA content |
| | 2 weeks | | after storage | 12 months | | after storage |
| | (mg/g powder) | | at 40° C. (%) | (mg/g powder) | | at 25° C. (%) |
| | Before | After | After | Before | After | After |
| Sample | storage | storage | storage | storage | storage | storage |
| Algal oil | 436.7 ± 14.6 | 127.0 ± 3.5 | 29.1 | 436.7 ± 14.6 | NA** | NA** |
| 80% broccoli | 39.4 ± 0.1 | 33.1 ± 1.2 | 84.1 | 39.4 ± 0.1 | 24.7 ± 1.6 | 62.8 |
| puree + 10% | | | | | | |
| algal oil + 10% | | | | | | |
| LGG | | | | | | |

Note*:
The BP-AO-LGG powder contains 10% algal oil.
NA** means FAME data is not available from the oil samples as the neat algal oil samples were gelled after the storage.

In one example, a commercial product produced by the methods described herein, will be nitrogen flashed and stored in a 3 layers of aluminium foil-laminated package with a water activity of about 0.2 or less at room temperature prior to use.

The stability of algal oil in the broccoli powders with LGG cells (BP-AO-LGG) against oxidation were determined using the apparatus ML OXIPRES (manufactured by Mikrolab Aarhus A/S, Hojbjerg, Denmark). The bulk algal oil (AO) was included as the control to see the end of induction period more evident than in the powders. The induction period was calculated as the time after which the pressure began to decrease sharply (the end was measured from the cross-section point of lines of the first part and the subsequent part of the curve recording the pressure changes, as shown in FIG. 22). The graph shown in FIG. 22 represent the relative oxidative stability of the oils, with the longer induction period being associated with greater protection afforded by the broccoli matrix to the oil under accelerated conditions. The results show that the pressure of AO sample began to decrease sharply around 6 hour of the reaction time whereas that of BP-AO-LGG sample was seen to be stable more than 20 h highlighting BP-AO-LGG matrix was protected against oxidation.

### Example 8

### Biomasses as Encapsulants

Raw biomass was cut into small pieces, boiling water was added initially and the mixture was blended to obtain an aqueous suspension containing the biomass (5% TS). The pH of this mixture was adjusted to 7.50 using 2N NaOH. Then the mixture was heat treated at 75° C. for 2 min or at 100° C. for 30 min then cooled down to 60° C. Omega-3 oil (tuna oil) was added (1:1 biomass solids:oil ratio) into the aqueous phase suspension (60° C.), and homogenised using an Ultraturrax at 15,000 rpm for 3 min to prepare the emulsions which were freeze dried to obtain powders (50% oil).

The results are provided in Table 12. These results show the relative oxidative stability of the oils, with the longer induction period being associated with greater protection afforded by the encapsulant to the oil under accelerated conditions.

TABLE 12
Oxidative stability of omega-3 oil (tuna
oil) powders (25% and 50% oil content).

| | | 50% Oil Powder | | 25% Oil Powder | |
|---|---|---|---|---|---|
| | | IP | Slope | IP | Slope |
| Encap- | | (hr) at | (−mBar/ | (hr) at | (−mBar/ |
| sulant | Treatment | 80° C. | hr) | 80° C. | hr) |
| Carrot | 75° C., 2 min | >20 | **, n/a | n/a | n/a |
| Carrot | 100° C., 30 min | >20 | **, n/a | n/a | n/a |
| Tomato | 75° C., 2 min | >20 | **, n/a | n/a | n/a |
| Tomato | 100° C., 30 min | >20 | **, n/a | n/a | n/a |
| Mushroom | 75° C., 2 min | >170 | n/a | Not | Not |
| | | | | done | done |
| Mushroom | 100° C., 30 min | >170 | n/a | >170 | n/a |
| Cauliflower | 75° C., 2 min | 80.5 | −237 | >88 | n/a |
| Kale | 75° C., 2 min | >100 | n/a | >144 | n/a |
| Brussel | 75° C., 2 min | 165 | −25 | >300 | n/a |
| sprouts | | | | | |
| Snow Peas | 75° C., 2 min | >160 | **, n/a | >160 | n/a |
| Garlic | 75° C., 2 min | >46 | **, n/a | 38 | −2640 |

**Sudden increase in pressure at IP leading to release of volatiles,
n/a—not applicable as not possible to obtain rate of oxygen uptake due to lack of distinctive IP; 50% oil powder (8 g powder, 4 g oil tested), 25% oil powder (12 g powder, 3 g oil tested); na—not tested.

### Example 9

### Survival of Lactic Acid Bacteria (LAB) in Sterilised Broccoli and Sterilised Broccoli-Algal Oil Matrices During Freeze Drying

### Methods

Preparation of powders: Bifidobacterium animalis subsp. lactis from Chr-Hansen, and Lactobacillus plantarum ATCC 8014 were used as the model probiotic bacteria that was carried in the broccoli-based matrices. Broccoli puree were prepared as follows. Whole broccoli was washed in clean water and then cut into small pieces. The broccoli pieces (10% total solids, according to previous experiment) was mixed with water at a ratio of 3:2 (broccoli:water) and homogenised using a food blender to produce broccoli puree (˜6% total solids). After homogenisation, the broccoli puree was autoclaved at 121° C. for 3 min for sterilisation. The sterilised broccoli puree was then cooled down at <10° C. and used as soon as possible for preparing broccoli emulsions/mixtures. The broccoli puree (BP) was mixed with algal oil, AO (10%) for 5 min at maximum speed using a Silverson emulsifier-mixer to obtain BP-AD emulsion. The LAB (10%) (Bifidobacterium animalis and Lactobacillus plantarum) was added in BP-AO emulsion and mixed for 30s to prepare BP-AO-LAB emulsion.

The following powder formulations were prepared using broccoli puree (BP): (a)

BP-Bifido powder: 90% BP and 10% Bifido culture preparation (dry basis), (b) BP-AO-Bifido powder: 80% BP, 10% algal oil and 10% Bifido culture preparation (dry basis), (c) BP-L. plantarum powder: 90% BP and 10% L. plantarum culture preparation (dry basis) (6% w/w bacterial suspension in 8% w/v glucose solution), (d) BP-AO-L. plantarum powder: 80% BP, 10% algal oil and 10% L. plantarum culture preparation (dry basis) (6% w/w bacterial suspension in 8% w/v glucose solution).

Viability after freeze drying: Enumeration of the bacteria was carried out using a standard plate count method. Freeze-dried probiotic powders were rehydrated by dispersing in Buffered peptone water (BPT) in a shaking water bath (37° C., 100 rpm, 1 h). The rehydrated samples were then diluted with Maximum recovery diluent (MRD). De Man, Rogosa and Sharpe agar (MRS agar, Oxoid Ltd, UK) was used for enumeration L. plantarum from the samples. RCA (reinforced clostridial, pH 6.8, Oxoid Ltd, UK) agar was used for enumeration of Bifidobacterium lactis from the samples. The inoculated plates were incubated under anaerobic conditions at 37° C. for 48 h. Each viable unit (cells) grown as a colony on the plates was counted as a colony forming unit (CFU) and calculated for the number of CFU per gram of the powder (CFU/g). The viable counts are transformed into logio value and loss of the count in the powders were calculated and compared with the control probiotic powders as well as with the value of each powder.

### Results

The initial viable counts of the samples and the viable counts after the powders production are given in Table 13. Before freeze drying, the broccoli with Bifodo powders had 4.60E+11 and 1.43E+11 colony forming unity (CFU)/g powder respectively for BP-Bifido and BP-AO-Bifido. After freeze-drying, the viable count cells in the BP-Bififo powder (90% Broccoli puree+10% Bifido) was 1.05E+10 CFU/g powder and that of BP-AO-Bifido (80% Broccoli puree+10% Algal oil+10% LGG) was 1.00E+09 CFU/g powder. This represents a loss of 1-2 log CFU/g during freeze-drying process. The less loss in BP-Bifido compared to that of BP-AO-Bifido during the process can be explained by the higher biomass content protected the cells from freeze-drying stress. However, there was no significant loss found in the broccoli with L. plantarum powders after freeze-drying. In the preparation of the broccoli-L. plantarum powder, harvested washed cells were dispersed in glucose solution (6% w/v bacterial suspension in 8% w/v glucose solution) before mixing to the broccoli matrices. Glucose solution may allow reducing the harmful effect of ice crystals of bacteria which can damage cells by dehydration caused by a localized increase in salt concentration leading to denaturation of proteins. This suggests that glucose solution should be added as a cryoprotectant which can depress the freezing point of bacterial cells by disrupting the crystal lattice formation of ice unless the temperature is significantly lowered.

### Example 10

### Survival of Lactic Acid Bacteria (LAB) in Sterilised Broccoli and Sterilised Broccoli-Algal Oil Matrices During Storage

### Methods

Freeze-dried broccoli powders (BP-LAB and BP-AO-LAB) and dried LAB culture preparation without the broccoli matrix were stored in open containers inside a desiccator at 25° C. with saturated salt solutions of magnesium chloride (MgCl₂) or magnesium nitrate (Mg (NO₃)₂) to provide atmospheres with relative humidity, RH of 32% (0.30 aw) and 52% (0.50 aw), respectively. The viable counts were obtained prior to and after storage.

TABLE 13
Survival of LAB after freeze-drying for the powder production

| | Viable LAB count/g powder | | Loss of |
|---|---|---|---|
| | (log₁₀ transformed value) | | LAB (log₁₀ |
| | Before | After | transformed |
| Sample | freeze-rying | freeze-rying | value) |
| 90% BP + 10% Bifido | 11.66 ± 0.10 | 10.01 ± 0.12 | 1.64 |
| 80% BP + 10% AO + | 11.14 ± 0.14 | 9.00 ± 0.00 | 2.14 |
| 10% bifido | | | |
| 90% BP + 10% | 9.78 ± 0.19 | 9.39 ± 0.55 | 0.39 |
| L. plantarum | | | |
| 80% BP + 10% AO + | 10.04 ± 0.20 | 9.75 ± 1.05 | 0.30 |
| 10% L. plantarum | | | |

Note:
For L. plantarum samples, sugar was added as cryoprotectant whereas there was no cryoprotectant in bifido samples.

### Results

The initial viable counts of the samples and the viable counts after 12-week storage of the powders at 25° C. at different water activities (0.3 and 0.5 aw) are given in Table 14. After the storage, the survival rate of all probiotic LAB strains was higher in the BP alone matrix (90% of BP) compared to the count in the BP+AO matrix (80% BP) and LAB cells without the matrix. The survival was seen to be higher under 0.3_{aw} than the survival under 0.5_{aw}.

### Example 11

### Survival of LAB Bacteria Carried by Sterilised Broccoli and Sterilised Broccoli-Algal Oil Matrices During In-Vitro Digestion

### Methods

Powders (BP-LAB powder: 90% BP and 10% LAB culture preparation (dry basis); BP-algal oil-LAB powder: 80% BP, 10% algal oil and 10% LAB culture preparation (dry basis) and LAB culture preparation) were used for assessment of viability after sequential exposure to simulated gastric fluid (SGF) and simulated intestinal fluids (SIF) as described in Example 6. The survival or viability of the LAB cells with/without broccoli matrices in simulated digestive fluids (SGF, and SIF) was performed by CFU pate counting as described in Example 9.

TABLE 14
Survival of LAB cells without broccoli matrices and LAB bacteria
with broccoli-based matrices after storage at 25° C.
at 32% RH (0.3 aw) and 52% RH (0.5 aw) for 12 weeks.

| | | Loss of LAB compared to | |
|---|---|---|---|
| | | Freeze-dried powder | |
| | Viable count/g | before storage | |
| | powder (log₁₀ | (log₁₀ transformed value) | |
| | transformed value) | After 12-week storage | |
| Sample | Before storage | 0.30 a_{w} | 0.50 |
| Bifido | 10.41 ± 0.10 | 6.06 | 6.17 |
| 90% BP + 10% Bifido | 10.01 ± 0.12 | 4.82 | 5.08 |
| 80% BP + 10% | 9.00 ± 0.00 | 3.67 | 4.50 |
| AO + 10% Bifido | | | |
| L. plantarum | 9.91 ± 0.19 | NA | NA |
| 90% BP + 10% | 9.39 ± 0.55 | 3.87 | 5.92 |
| L. plantarum | | | |
| 80% BP + 10% AO + | 9.75 ± 1.05 | 3.94 | 5.25 |
| 10% L. plantarum | | | |

NA means not available.

### Results

Simulated in-vitro digestion of the samples containing LAB by sequential exposure of samples to simulated gastric and intestinal fluids (SGF+SIF). Survival of LAB cells in the in-vitro digested samples were plate counted. The initial LAB counts of the LAB culture preparation (without the broccoli matrix) before and after (SGF+SIF), and the LAB counts of LAB-loaded broccoli matrices before and after (SGF+SIF) are given in Table 15. Overall, probiotic survival was higher in the BP alone matrix (90% of BP) compared to LAB count in the BP+AO matrix (80% BP) and LAB cells without the matrix. In brief, the probiotics strains behaved differently to the in-vitro digestion processes. Bifido and L. plantarum alone without broccoli matrix had up to 4.48 and 5.71 log10 (CFU/g) loss after the digestion. The loss of L. plantarum with the BP matrix were around 3 log10 less loss compared to L. plantarum alone after the digestion. However, the broccoli matrix did not offer benefit on the survival of Bifido cells since the log10 loss difference were less than 1 compared to Bifido alone.

TABLE 15
Survival of LAB cells without the broccoli matrix and survival
of total LAB in LAB-loaded broccoli matrices upon sequential
exposure to simulated gastric fluid (SGF) for 2 h and
simulated intestinal fluid (SIF) for 3 h.

| | | | Loss of LAB |
|---|---|---|---|
| | Viable count/g powder | | compared to |
| | (log₁₀ transformed value) | | Freeze-dried |
| | Before | After | powder (log₁₀ |
| | in-vitro | in-vitro | transformed |
| Sample | digestion | digestion | value) |
| Bifido | 10.41 ± 0.10 | 5.93 ± 0.07 | 4.48 |
| 90% BP + 10% | 10.01 ± 0.12 | 6.09 ± 0.55 | 3.93 |
| Bifido | | | |
| 80% BP + 10% | 9.00 ± 0.00 | 4.72 ± 0.09 | 4.28 |
| AO + 10% Bifido | | | |
| L. plantarum | 9.91 ± 0.19 | 4.20 ± 0.23 | 5.71 |
| 90% BP + 10% | 9.39 ± 0.55 | 6.47 ± 0.10 | 2.92 |
| L. plantarum | | | |
| 80% BP + 10% AO + | 9.75 ± 1.05 | 7.00 ± 0.06 | 3.75 |
| 10% L. plantarum | | | |

### Example 12

### Oxidative Stability of Algal Oil in Sterilised Broccoli-Algal Oil-LAB Delivery Systems

### Methods

The broccoli-algal oil-LAB (BP-AO-LAB) powder: 80% BP, 10% algal oil and 10% LGG culture preparation (dry basis) prepared as described in Example 9 above and algal oil were stored at 25° C. for 6 months. The fatty acid composition of the algal oil and broccoli powder (BP-AO-LAB: powder containing 80% broccoli puree+10% algal oil+10% LAB) and the % remaining docosahexaenoic acid (DHA) in the stored samples (25° C. for 5 months) were determined. As described in Example 7, Fatty acid composition was determined by gas chromatography (GC) after preparation of fatty acid methyl esters (FAME).

### Results

The DHA content and calculated remaining amounts (%) of the content of BP-AO-LAB powder compared to the algal oil are shown in Table 16. After 6 months storage at 25° C., it was noticed that the stored neat algal oil samples had deteriorated significantly and formed a gel and FMAE on that sample was not performed. However, the AO within the BP-AO matrix was protected against oxidation where the % DHA remaining after the storage was 94.2%. The remaining % of DHA in the stored BP-AO-LAB samples was in the range of 73.2-94.7% where that in the BP-AO-LGG sample was the highest % DHA remaining. The results highlighted that the combination of BP-AO matrix with LGG provided more than 90% of AO against oxidation during the storage.

TABLE 16
Stability of docosahexaenoic acid (DHA) in sterilised
broccoli-algal oil-LGG powder compared to algal oil
(before and after the storage at 25° C. for 5 months).

| | | | Remaining |
|---|---|---|---|
| | | | DHA content |
| | DHA content at 25° C. | | after storage |
| | for 5 months (mg/g powder) | | at 25° C. (%) |
| | Before | After | After |
| Sample | storage | storage | storage |
| Algal oil | 421.3 ± 1.6 | NA** | NA** |
| 90% broccoli | 33.4 ± 1.0 | 31.5 ± 0.4 | 94.2 |
| puree + 10% | | | |
| algal oil | | | |
| 80% broccoli | 30.7 ± 0.6 | 29.1 ± 1.1 | 94.7 |
| puree + 10% | | | |
| algal oil+ | | | |
| 10% LGG | | | |
| 80% broccoli | 31.3 ± 0.5 | 27.6 ± 0.6 | 88.2 |
| puree + 10% | | | |
| algal oil + | | | |
| 10% Bifido | | | |
| 80% broccoli | 25.9 ± 3.1 | 19.0 ± 0.3 | 73.2 |
| puree + 10% | | | |
| algal oil + | | | |
| 10% L. plantarum | | | |

Note*:
The BP-AO-LAB powder contains 10% algal oil.
NA** means FAME data is not available from the oil samples as the neat algal oil samples were gelled after the storage

### Example 13

### Survival of Lactic Acid Bacteria (LAB) in Other Green Banana and Sweet Potato and Green Banana and Sweet Potato-Algal Oil Matrices During Freeze Drying

### Preparation of Vegetable Puree or Vegetable-AO Emulsion Comprising Formulations

Probiotics' survival using other matrices (i.e. green banana and sweet potato) during freeze drying and GI transit was investigated. The choice of green banana is based on the high protein contents (4.0%) and 40% resistant starch dry weight from previous studies undertaken within the same research group. Sweet potato has protein: 1.6% carbs: 20.1%, sugar: 4.2%, fiber: 3%. Sweet potato has health benefit including promote gut-health & immune system and has anticancer properties.

Green banana flour (93.8% total solids; Macro green banana flour obtained from Woolworths) was mixed with water at a ratio of 1:18 (green banana: water) to produce green banana puree (˜5.2% total solids). The mixture was hydrated and homogenised at 50° C. for 1 hr. The green banana puree (GB) was homogenised with algal oil, AO (25%) for 5 min at maximum speed using a Silverson emulsifier-mixer to produce an emulsion of GB and AO (GB-AO). To prepare GB-AO-LGG emulsion, LAB (5% LGG) was added in GB-AO emulsion (according to the dry weight content shown in Table 17) and mixed for further 30 s.

Similar preparations were done for sweet potato puree and emulsions. The sweet potato flour (93.6% total solids; Macro Sweet Potato Flour obtained from Woolworths) was mixed with water at a ratio of 1:18 (sweet potato: water) to produce sweet potato puree (˜5.2% total solids). Sweet potato puree (SP), SP-A0 emulsion and SP-AO-LGG emulsion were prepared as above. Formulations comprising one or more of LGG, GB, AO, and SP were prepared for the studies, the composition and amounts based on dried weight is provided in Table 17. All emulsions and mixtures prepared were freeze-dried into powder and stored at −20° C. before analysis.

TABLE 17
Formulation of green banana (GB), sweet potato (SP), algal
oil (AO), probiotics LGG and combinations thereof.

| | Vegetable | | |
|---|---|---|---|
| | powder (g, | AO | LGG |
| Formulation* | dry matter) | (g) | (g) |
| Green banana (GB) | 100 | — | — |
| 75% GB-25% AO (Algal oil) | 75 | 25 | — |
| 70% GB-25% AO-5% LGG | 70 | 25 | 5 |
| Sweet potato (SP) | 100 | — | — |
| 75% SP + 25% AO (Algal oil) | 75 | 25 | — |
| 70% SP-25% AO-5% LGG | 70 | 25 | 5 |

*0.15 g of each formulation was used for in-vitro colonic fermentation.

### Survival of LAB Cells in Vegetable Biomass and Vegetable Biomass-Algal Oil Matrices During Freeze Drying

### Methods

The survival of viability of the LAB cells in the vegetable biomass-algal oil matrices during freeze drying was performed by CFU pate counting as described in Example 4.

### Results

Before freeze drying, the GB-AO-LGG (70% green banana puree+25% algal oil+5% LGG) and SP-AO-LGG (70% sweet potato puree+25% algal oil+5% LGG) powders had 6.43E+08 CFU/g powder (colony forming unity/g powder) and 2.12E+09 CFU/g powder respectively. After freeze-drying, the viable count cells in the GB-AO-LGG powder was 1.44E+08 CFU/g powder and that of SP-AO-LGG was 1.98E+08 CFU/g powder. This represents a loss of about or less than 1 log CFU/g.

### Example 14

### Survival of Lactic Acid Bacteria (LAB) in Other Green Banana and Sweet Potato Matrices and Green Banana and Sweet Potato Biomass-Algal Oil Matrices During In-Vitro Digestion

### Methods

The survival of viability of the LAB cells in the vegetable biomass-algal oil matrices during in-vitro digestion was performed by CFU pate counting as described in Example 6.

### Results

Simulated in vitro digestion of the samples containing LGG was performed by sequential exposure of samples to simulated gastric and intestinal fluids (SGF+SIF). Survival of LGG cells in the in vitro digested samples were plate counted. The initial LGG counts of the LGG culture preparation (without the vegetable matrix) before and after (SGF+SIF), and the total LAB counts of LGG-loaded vegetable matrices before and after (SGF+SIF) are given in Table 18. Higher probiotic survival rate was found in the vegetable matrix (70% of GB or SP) compared to LAB cells without the matrix.

TABLE 18
Survival of LAB cells without the green banana and sweet potato matrix
and survival of total LAB in LAB-loaded green banana and sweet potato
matrices upon sequential exposure to simulated gastric fluid (SGF)
for 2 h and simulated intestinal fluid (SIF) for 3 h.

| | | | Loss of LAB |
|---|---|---|---|
| | Viable count/g powder | | compared to |
| | (log₁₀ transformed value) | | Freeze-dried |
| | Before | After | powder (log₁₀ |
| | in-vitro | in-vitro | transformed |
| Sample | digestion | digestion | value) |
| LGG | 12.2 ± 0.36 | 6.2 ± 0.57 | 6.1 |
| 70% GB + 25% | 8.1 ± 0.28 | 4.2 ± 0.43 | 3.9 |
| AO + 5% LGG | | | |
| 70% SP + 25% | 8.3 ± 0.07 | 5.1 ± 0.16 | 3.2 |
| AO + 5% LGG | | | |

### Example 15

### Oxidative Stability of Algal Oil in Vegetable Matrix-Algal Oil-LAB Delivery Systems

### Methods

The vegetable biomass-algal oil powders (GB-AO and SP-AO): 75% GB or SP, 25% algal oil (dry basis) prepared as described in Example 14 above and algal oil were stored at 40° C. for 2 weeks. The fatty acid composition of the algal oil and vegetable powders and the % remaining docosahexaenoic acid (DHA) in the stored samples (40° C. for 2 weeks) were determined. As described in Example 7, Fatty acid composition was determined by gas chromatography (GC) after preparation of fatty acid methyl esters (FAME).

### Results

The DHA content and calculated remaining amounts (%) of the content of GB-AO and SP-AO powder compared to the algal oil are shown in Table 19. After 2 weeks storage at 40° C., the % DHA remaining in the stored algal oil samples decreased to 29.1% where that in the GB-AO & SP-AO samples were 73.2% and 77.9% respectively. The results highlighted that the AO within the matrix was protected against oxidation compared to when the neat oil was stored.

TABLE 19
Stability of docosahexaenoic acid (DHA) in vegetable
biomass-algal oil powder compared to algal oil (before
and after the storage at 40° C. for 2 weeks).

| | | | Remaining |
|---|---|---|---|
| | DHA content at | | DHA content |
| | 40° C. for 2 weeks | | after storage |
| | (mg/g powder) | | at 40° C. (%) |
| | Before | After | After |
| Sample | storage | storage | storage |
| Algal oil | 440.3 ± 19.1 | 128.1 ± 4.2 | 29.1 |
| 75% GB + 25% AO | 111.7 ± 3.2 | 81.8 ± 6.7 | 73.2 |
| 75% SP + 25% AO | 76.8 ± 0.1 | 59.8 ± 1.0 | 77.9 |

Note*:
The BP-AO-LAB powder contains 25% algal oil.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Persons skilled in the art will appreciate that the comparisons of the extent of protection afforded to the actives (polyunsaturated oils and/or probiotic) by incorporation in the encapsulant matrices during storage are assessed under accelerated shelf-life conditions (i.e. high temperature, oxygen atmosphere and water activity) and are indicative of the protection that is offered by encapsulation. In commercial applications, when proper packaging and storage conditions are used, there will also be protection afforded to the actives.

This application claims priority from Australian Provisional Application No. 2019902828 entitled “Compositions and methods for promoting health in a subject” filed on 7 Aug. 2019, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Agerbirk and Olsen (2012) Phytochemisty 77:16-45.
Alvarez-Sieiro et al. (2016) Applied Microbiology and Biotechnology 7:2939-2951.
Chaucheyras-Durand and Durand (2010) Beneficial Microbes 1: 3-9.
Halkier and Gershenzon (2006) Annual Review in Plant Biology 57:303-33.
Huynh et al. (2017) Fish and Shellfish Immunology 64:367-382
Koh et al. (2016) Cell 165: 1332-1345.
Krishna and Samak et al. (2013) Current Nutrition & Food Science 9: 99-107.
LeBlanc et al. (2017) Microbial Cell Factories 16: 79.
Markowiak and Śliżewska (2017) Nutrients 9: 1021.
Markowiak and Śliżewska (2018) Gut Pathogens 10: 21.
Martínez Cruz et al. (2012) International Scholarly Research Network Microbiology 2012:916845.
Verkerk et al. (2009) Molecular Nutrition and Food Research 53:5219-S265.
Walker et al. (2005) Applied and Environmental Microbiology 71: 3692-3700.

1. A powder composition comprising a probiotic entrapped or encapsulated in a matrix comprising protein and carbohydrate from a non-fermented biomass from a single species of organism.

2. The composition of claim 1, wherein the entrapped or encapsulated probiotic has higher viability compared to the unentrapped or unencapsulated probiotic.

3. The composition of claim 1 or claim 2, wherein the entrapped or encapsulated probiotic has higher viability compared to the unentrapped or unencapsulated probiotic after treatment with/in one or more or all of the following conditions:
i) a temperature of about 4° C. to about 40° C.;
ii) a pH of about 1 to about 7;
iii) a pH of about 1 to about 5;
iv) simulated gastric fluid;
v) gastric fluid;
vi) digestive enzymes;
vii) simulated intestinal fluid;
viii) intestinal fluid;
ix) transit through the upper gastrointestinal tract;
x) the lower gastrointestinal tract;
xi) freeze drying; and
xii) sterilization.

4. The composition of any one of claims 1 to 3, wherein the composition is synbiotic.

5. The composition of any one of claims 1 to 4, wherein the probiotic is one or more of the following:
i) a beneficial bacteria;
ii) a lactic acid bacteria;
iii) a bacteria that produces one or more short chain fatty acid/s (SCFA) when in the gastrointestinal tract;
iv) a bacteria that assists with the production of one of more short chain fatty acid/s (SCFA) in the gastrointestinal tract;
v) isolated from a Brassicaceae;
vi) isolated from broccoli;
vii) isolated from Daucus carota; and
viii) an autochonthous bacteria from the biomass.

6. The composition of claim 5, wherein the lactic acid bacteria is selected from one or more of the genera selected from: Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus and Weissella.

7. The composition of claim 5 or claim 6, wherein the lactic acid bacteria is selected from one or more of: Leuconostoc mesenteroides, Lactobacillus plantarum, Lactobacillus pentosus, Lactobacillus brevis, Lactococus lactis, Pediococcus pentosaceus, Lactobacillus rhamnosus, Pedicoccus acidilacti, BF1 deposited under V17/021729 on 25 Sep. 2017 at the National Measurement Institute 1/153 Bertie Street, Port Melbourne, Victoria, Australia 3207, BF2 deposited under V17/021730 on 25 Sep. 2017 at the National Measurement Institute 1/153 Bertie Street, Port Melbourne, Victoria, Australia 3207, B1 deposited under V17/021731 on 25 Sep. 2017 at the National Measurement Institute 1/153 Bertie Street, Port Melbourne, Victoria, Australia 3207, B2 deposited under V17/021732 on 25 Sep. 2017 at the National Measurement Institute 1/153 Bertie Street, Port Melbourne, Victoria, Australia 3207, B3 deposited under V17/021733 on 25 Sep. 2017 at the National Measurement Institute 1/153 Bertie Street, Port Melbourne, Victoria, Australia 3207, B4 deposited under V17/021734 on 25 Sep. 2017 at the National Measurement Institute 1/153 Bertie Street, Port Melbourne, Victoria, Australia 3207, and B5 deposited under V17/021735 on 25 Sep. 2017 at the National Measurement Institute 1/153 Bertie Street, Port Melbourne, Victoria, Australia 3207.

8. The composition of claim 5, wherein the bacteria that produces one or more SCFA is selected from one or more of: Faecalibacterium prausnitzii, Roseburia intestinalis, Roseburia faecis, Roseburia inulinivorans, Eubacterium rectale, Eubacterium halii, Anaerostipes hadrus, Anaerostipes caccae, Butyrivibrio crossotus, Clostridium cluster XIVa. Bifidobacterium spp., Bacteroidetes and Negativicutes classes of Firmicutes.

11. The composition of any one of claims 1 to 10, wherein the oil is entrapped or encapsulated in the matrix, and wherein the entrapped or encapsulated oil is resistant to degradation compared to the unentrapped or unencapsulated oil.

12. The composition of claim 10 or claim 11, wherein the oil is resistant to degradation by one or more of: oxygen, temperature, pH, moisture, light, and pro-oxidants.

13. The composition of any one of claims 10 to 12, wherein the oil is resistant to degradation at one or more of the following conditions:
i) for at least one week when stored at about 40° C.;
ii) for at least two weeks when stored at about 40° C.;
iii) for at least 5 months when stored at about 24° C.; and
iv) for at least 12 months when stored at about 24° C.

14. The composition of any one of claims 10 to 13, wherein the oil comprises one or more fatty acid/s.

15. The composition of any one of claims 10 to 14, wherein the fatty acid is selected from one or more of: omega-3, omega-6 or an omega-9 fatty acid.

16. The composition of claim 15, wherein the omega-3 fatty acid is one or more of α-linolenic acid (ALA), eicosapentaenoic acid (EPA), docosapentaenoic acid (DPA) and docosahexaenoic acid (DHA).

17. The composition of any one of claims 10 to 16, wherein the oil is selected from one or more of: fish oil, hill oil, algal oil, marine oil, fungal oil, nut or seed oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, medium chain triglycerides and grapeseed oil.

18. The composition of claim 17, wherein the fish oil or marine oil is selected from one or more of: tuna oil, herring oil, mackerel oil, sardine oil, cod liver oil, menhaden oil, shark oil, algal oil, squid oil, and squid liver oil.

19. The composition of any one of claims 1 to 18, wherein the composition comprises a further non-fermented biomass comprising protein and carbohydrate from at least one further single species of organism.

20. The composition of any one of claims 1 to 19, wherein one or more components of the biomass or further biomass is a prebiotic.

21. The composition of any one of claims 1 to 20, wherein the biomass or further biomass is from the Plantae or Fungi Kingdom.

22. The composition of claim 21, wherein the Plantae is selected from: Brassicaceae, Musaceae, Convolvulaceae, Cannabis, Asparagaceae, Arecaceae, Myrtaceae, Rosaceae, Ericaceae, Saxifragaceae, Cucurbitaceae, Nightshade, Capparaceae, Adoxaceae, Vitaceae, Rutaceae, Actinidiaceae, Sapindaceae, Anacardiaceae, Moraceae, Oleaceae, Cactaceae, Passifloraceae, Bromeliaceae, Cactaceae, Lythraceae, Polygonaceae, Cucurbitaceae, Oxalidaceae, Caesalpinioideae, Compositae, Amaranthaceae, Chenopodiacae, Malvaceae, Amarylidaceae, Fabaceae, Arecaceae and Poaceae.

23. The composition of claim 22, wherein the Plantae is Brassicaceae.

24. The composition of claim 23, wherein when the Plantae is:
i) Brassicaceae the Brassicaceae is broccoli;
ii) Musaceae the Musaceae is green banana; and
iii) Convolvulaceae the Convolvulaceae is sweet potato.

25. The composition of any one of claims 1 to 24, wherein the composition comprises one or more or all of:
i) at least one serving of vegetable,
ii) at least one serving of fruit,
iii) at least one serving of omega-3,
iv) at least one serving of omega-6,
v) at least one serving of omega-9, and
vi) at least one serving of probiotic.

26. The composition of any one of claims 1 to 25, wherein the composition increases the gastrointestinal level of one or more short chain fatty acid/s (SCFA) in a subject.

27. The composition of claim 26, wherein the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoi acid), isobutyric acid (2-methylpropanoic acid), valeric acid (pentanoic acid), isovaleric acid (3-methylbutanoic acid), and caproic acid (hexanoic acid).

28. The composition of claim 26 or claim 27, wherein the SCFA is selected from one or more or all of: butyrate, propionate and acetate.

29. The composition of any one of claims 1 to 28, wherein the composition increases the gastrointestinal level of one or more of:
i) lactic acid bacteria,
ii) a bacteria that produces one or more SCFA; and
iii) a bacteria that assists with the production of one of more SCFA.

30. The composition of any one of claims 1 to 29, wherein the composition comprises about 50% to about 90%, or about 60% to about 80%, or about 70% to about 80%, biomass.

31. The composition of any one of claims 1 to 30, wherein the composition comprises about 1×10⁶ CFU/g to about to 1×10¹² CFU/g, or about 1×10⁷ CFU/g to about to 1×10¹¹ CFU/g, about 1×10⁸ CFU/g to about to 1×10¹⁰ CFU/g, and 1×10⁹ CFU/g to about to 1×10¹⁰ CFU/g, probiotic.

32. The composition of any one of claims 1 to 31, wherein the composition comprises about 5% to about 50%, or about 10% to about 45%, or about 10% to about 40%, or about 15% to about 30%, or about 20% to about 30% w/w, oil.

33. The composition of any one of claims 1 to 32, wherein the composition increases the gastrointestinal level of one or more bacteria selected from: Colinsella, Bacillus, Lactobacillus, Lachnospira, Faecalibacterium, Dialister and Veillonella in a subject.

34. The composition of any one of claims 1 to 33, wherein the composition decreases the gastrointestinal level of one or more or all of: Bacteroides, Parabacteroides, Paraprevotella, Turicibacter, Christensenellaceae, some members of Family Clostridiales, Clostridium, some members of Family Lachnospiraceae, Dorea, Roseburia, some members of the Family Ruminococcaceae, Oscillospira, Ruminococcus, some members of the Family Erysipelotrichaceae, Klebsiella and Trabulsiella in a subject.

35. A method of producing a powder composition, the method comprising
i) producing an aqueous mixture comprising
a) protein and carbohydrate from a non-fermented biomass from a single species of organism, and
b) a probiotic, and
ii) forming, from the mixture, a powder comprising the probiotic entrapped or encapsulated in a matrix comprising the protein and the carbohydrate.

36. The method of claim 35, further comprising adding oil to the aqueous mixture.

37. The method of claim 35 or claim 36, wherein the method comprises adding protein and carbohydrate from at least one further non-fermented biomass from a single species of organism.

38. The method of any one of claims 35 to 37, further comprising pre-treating the biomass.

39. The method of claim 38, wherein pre-treating comprises one or more of:
i) heating;
ii) macerating;
iii) microwaving;
iv) exposure to low frequency sound waves (ultrasound);
v) pulse electric field processing;
vi) static high pressure;
vii) extrusion;
viii) enzyme treatment;
ix) an extraction or separation process; and
x) drying.

40. The method of any one of claims 35 to 39, wherein the composition is adjusted to a pH of about 4.5 to about 7.5, or a pH of about 5 to about 7, or a pH of about 5.5. to about 6.8, or a pH of about 6 to about 6.8, or a pH of about 6.8.

41. A product comprising the composition of any one of claims 1 to 34, or produced by the method of any one of claims 35 to 40.

42. The product of claim 41, wherein the product is synbiotic.

43. The product of claim 41 or claim 42, wherein the product comprises an isothiocyanate and/or glucosinolate.

44. The product of any one of claims 41 to 43 which is a cream, gel tablet, liquid, pill, powder or extruded product.

45. The product of claim 44, wherein the product is a powder.

46. The product of claim 45, wherein the powder has an induction period of about 10 to about 300 hours, when measured at 80° C. and a 5 bar initial oxygen pressure.

47. The product of any one of claims 41 to 46, wherein the product is a food, food ingredient or supplement.

48. The product of any one of claims 41 to 47, wherein the product comprises an omega 3 polyunsaturated fatty acid.

49. The product of any one of claims 41 to 48, wherein the product is selected from:
i) an animal feed,
ii) feed ingredient,
iii) supplement
iv) an aquaculture feed,
v) aquaculture feed ingredient, and
vi) aquaculture supplement.

50. The product of any one of claims 41 to 49, wherein the product does not comprise a dairy and/or an animal material.

51. A method of promoting health in a subject, comprising administering to the subject a composition of any one of claims 1 to 34, or product of any one of claims 35 to 40.

52. The method of claim 51, wherein the composition or product increases the gastrointestinal level of one or more short chain fatty acid/s (SCFA) in the subject.

53. The method of claim 52, wherein the SCFA is selected from one or more or all of: butyrate (butanoate), propionate (propanoate), acetate (ethanoate), formate (methanoate), isobutyrate (2-Methylpropanoate), valerate (pentanoate), isovalerate (3-methylbutanoate), caproate (hexanoate), formic acid (methanoic acid), acetic acid (ethanoic acid), propionic acid (propanoic acid), butyric acid (butanoi acid), isobutyric acid (2-methylpropanoic acid), valeric acid (pentanoic acid), isovaleric acid (3-methylbutanoic acid), and caproic acid (hexanoic acid).

54. The method of claim 52 or claim 53, wherein the SCFA is selected from one or more or all of: butyrate, propionate and acetate.

55. The method of any one of claims 51 to 54, wherein promoting health comprises promoting one or more of: gut health, immune system health, cardiovascular health, central nervous system function, cognition, metabolic health, nutrient absorption, nutrient utilisation, reducing acidosis, daily increase in body weight, an increase in total body weight, resistance to pathogen colonisation, skeletal health, liver health, blood sugar control and skin health.

56. The method of any one of claims 51 to 55, wherein promoting health comprises treating or preventing one or more symptoms of a condition selected from: diabetes, inflammation, metabolic dysfunction, asthma, allergy and cancer.

57. The method of claim 56, wherein promoting gut health comprises reducing or preventing one or more symptoms of a gut health associated condition selected from one or more of: irritable bowel syndrome, inflammatory bowel disease, Crohn's disease, colorectal cancer, gut leakiness, non-alcoholic fatty liver disease, metabolic syndrome, obesity, small intestinal bacterial overgrowth (SIBO), gastroenteritis, gut microbial dysbiosis, reduced gut microbial diversity, antibiotic treatment, post-surgery recovery, food intolerance, diarrhea, gastritis, diverticulitis, flatulence, constipation, functional gut disorders and functional gastrointestinal and motility disorders.

58. The method of any one of claims 51 to 57, wherein promoting health comprises promoting health of the gut microbiome in a subject.

59. The method of claim 58, wherein promoting health of the gut microbiome comprises one or more of:
i) increasing the level and/or activity of one or more beneficial bacteria,
ii) decreasing or maintaining the level and/or activity of one or more non-beneficial bacteria,
iii) increasing the resistance of the gut microbiome,
iv) increasing the resilience of the gut microbiome,
v) increasing the diversity of the gut microbiome,
vi) reducing gut leakiness, and
vii) increasing the production of one or more SCFA.

60. The method of claim 59, wherein the beneficial bacteria is lactic acid bacteria and/or a bacteria that produces one or more SCFA.

61. The method of claim 59, wherein the non-beneficial bacteria is selected from a pathogenic Bacteroides or Parabacteriodes.

62. The method of any one of claims 51 to 61, wherein promoting health of the gut microbiome comprises increasing the gastrointestinal level of one or more bacteria selected from: Colinsella, Bacillus, Lactobacillus, Lachnospira, Faecalibacterium, Dialister and Veillonella in a subject.

63. The method of any one of claims 51 to 62, wherein promoting health of the gut microbiome decreasing the gastrointestinal level of one or more or all of: Bacteroides, Parabacteroides, Paraprevotella, Turicibacter, Christensenellaceae, some members of Family Clostridiales, Clostridium, some members of Family Lachnospiraceae, Dorea, Roseburia, some members of the Family Ruminococcaceae, Oscillospira, Ruminococcus, some members of the Family Erysipelotrichaceae, Klebsiella and Trabulsiella in a subject.

64. The method of any one of claims 51 to 63, wherein the subject is an animal.

65. The method of any one of claims 51 to 63, wherein the subject is a human.

66. The method of any one of claims 51 to 64, wherein the subject is livestock or a companion animal.

67. The method of claim 66, wherein the livestock is selected from a: sheep, cow, goat, chicken, turkey, horse, donkey, pig, fish, prawn and shrimp.

68. A method of promoting the health of the gut microbiome in a subject, comprising administering to the subject the composition of any one of claims 1 to 34, or product of any one of claims 35 to 40.

69. A method of treating and/or preventing microbial dysbiosis in the gastrointestinal tract of a subject, comprising administering to the subject the composition of any one of claims 1 to 34, or product of any one of claims 35 to 40.

70. A method of treating and/or preventing inflammation in a subject, comprising administering to the subject the composition of any one of claims 1 to 34, or product of any one of claims 35 to 40.

71. A method of treating and/or preventing diabetes in a subject, comprising administering to the subject the composition of any one of claims 1 to 34, or product of any one of claims 35 to 40.

72. A method of promoting growth or feed efficacy in livestock comprising administering to a livestock the composition of any one of claims 1 to 34, or the product of any one of claims 35 to 40.

73. A method of improving the quality of livestock derived products comprising administering to a livestock the composition of any one of claims 1 to 34, or the product of any one of claims 35 to 40.

74. The method of claim 73, wherein the livestock derived product is milk, meat or eggs.

75. Use of a composition of any one of claims 1 to 34, or product of any one of claims 35 to 40 in the manufacture of a medicament for promoting health in a subject.

76. Use of a composition of any one of claims 1 to 34, or product of any one of claims 35 to 40 in the manufacture of a medicament for promoting health of the gut microbiome in a subject.

77. Use of a composition of any one of claims 1 to 34, or product of any one of claims 35 to 40 in the manufacture of a medicament for treating and/or preventing microbial dysbiosis in the gastrointestinal tract of a subject.

78. Use of a composition of any one of claims 1 to 34, or product of any one of claims 35 to 40 in the manufacture of a medicament for treating and/or preventing inflammation in a subject.

79. Use of a composition of any one of claims 1 to 34, or product of any one of claims 35 to 40 in the manufacture of a medicament for treating and/or preventing diabetes in a subject.

80. A pharmaceutical composition comprising a composition of any one of claims 1 to 34, or product of any one of claims 35 to 40 for use in promoting health in a subject.

81. A pharmaceutical composition comprising a composition of any one of claims 1 to 34, or product of any one of claims 35 to 40 for use in promoting health of the gut microbiome in a subject.

82. A pharmaceutical composition comprising a composition of any one of claims 1 to 34, or product of any one of claims 35 to 40 for treating and/or preventing microbial dysbiosis in the gastrointestinal tract of a subject.

83. A pharmaceutical composition comprising a composition of any one of claims 1 to 34, or product of any one of claims 35 to 40 for treating and/or preventing inflammation in a subject.

84. A pharmaceutical composition comprising a composition of any one of claims 1 to 34, or product of any one of claims 35 to 40 for treating and/or preventing diabetes in a subject.

85. The method or composition of any one of claims 68 to 84, wherein the composition or product is administered enterally.

86. The method or composition of claim 85, wherein administration is oral or rectal.

87. The method or composition of any one of claims 68 to 85, wherein the composition or product is administered topically.

88. Faecal microbiota suitable for transplantation into a subject, wherein the faecal microbiota has been isolated from a subject administered a composition of any one of claims 1 to 34, or a product of any one of claims 35 to 40.
